(19) United States
(12) Patent Application Publication
(10) Pub. No.: US 2023/0250109 A1
(43) Pub. Date: Aug. 10, 2023

(54) MACROCYCLIC ETHER CONTAINING INDOLE DERIVATIVES AS INHIBITORS OF MCL-1

(71) Applicant: Janssen Pharmaceutica NV, Beerse (BE)

(72) Inventors: Benoît Christian Albert Ghislain DE BOECK, Genval (BE); Petrus Jacobus Johannes Antonius BUIJNSTERS, Etten-Leur (NL); Sofia FERRER CABRERA, Beerse (BE); Aldo PESCHIULLI, Antwerp (BE); Tristan REUILLON, Molndal (SE); Frederik Jan Rita ROMBOUTS, Wilrijk (BE); Adriana-Ingrid VELTER, Antwerpen (BE)

(21) Appl. No.: 18/004,322

(22) PCT Filed: Jul. 8, 2021
(86) PCT No.: PCT/EP2021/069037

§ 371 Date: Jan. 5, 2023

(30) Foreign Application Priority Data
Jul. 8, 2020 (EP) 20184729.0

(51) Int. Cl. C07D 515/22; C07D 498/22; A61P 35/02

(52) U.S. Cl. CPC C07D 515/22; C07D 498/22; A61P 35/02

(57) ABSTRACT

The present invention relates to pharmaceutical agents useful for therapy and/or prophylaxis in a subject, pharmaceutical composition comprising such compounds, and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009; (113) 2805-2815).

WO2018178226 discloses MCL-1 inhibitors and methods of use thereof.

WO2017182625 discloses macrocyclic MCL-1 inhibitors for treating cancer.

WO2018178227 discloses the synthesis of MCL-1 inhibitors.

WO2020063792 discloses indole macrocyclic derivatives.

WO2020103864 discloses macrocyclic indoles as MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns novel compounds of Formula (I)

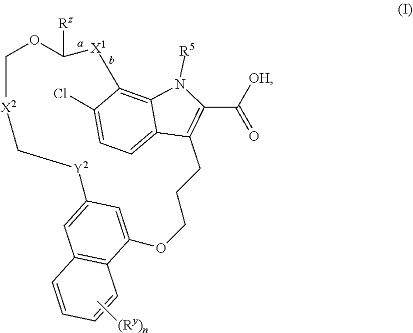

and the tautomers and the stereoisomeric forms thereof, wherein
X¹ represents

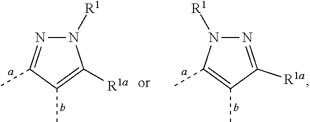

wherein ‘a’ and ‘b’ indicate how variable X¹ is attached to the remainder of the molecule;
R^{y} represents halo;
n represents 0, 1 or 2;
R^{z} represents hydrogen; or C₁₋₄alkyl optionally substituted with one Het¹;
X² represents

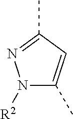

which can be attached to the remainder of the molecule in both directions;
R¹ represents hydrogen; Het^{a}; C₃₋₆cycloalkyl; or C₁₋₆alkyl optionally substituted with one or two substituents selected from the group consisting of Het¹, —OR³, and —NR^{4a}R^{4b};
R² represents hydrogen; methyl; or C₂₋₆alkyl optionally substituted with one substituent selected from the group consisting of Het¹, —OR³, and —NR^{4a}R^{4b};
R^{1a} represents methyl or ethyl;
R³ represents hydrogen, C₁₋₄alkyl, or —C₂₋₄alkyl-O—C₁₋₄alkyl;
R^{4a} and R^{4b} are each independently selected from the group consisting of hydrogen and C₁₋₄alkyl;
R⁵ represents methyl; or C₂₋₆alkyl optionally substituted with one substituent selected from the group consisting of C₃₋₆cycloalkyl, Het¹, —NR^{4a}R^{4b}, and —OR³;
Het¹ represents a 4- to 7-membered monocyclic fully saturated heterocyclyl containing one or two heteroatoms each independently selected from O, S, and N, wherein said S-atom might be substituted to form S(═O) or S(═O)₂; wherein said heterocyclyl is optionally substituted with one or two substituents each independently selected from the group consisting of halo, cyano, and —O—C₁₋₄alkyl;
Het^{a} represents a C-linked 4- to 7-membered monocyclic fully saturated heterocyclyl containing one heteroatom selected from O, S, and N, wherein said S-atom might be substituted to form S(═O) or S(═O)₂, and wherein said N-atom might be substituted with one C₁₋₄alkyl;
Y² represents —CH₂— or —S—;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₄alkyl’ as used herein as a group or part of a group represents a straight or branched chain fully saturated hydrocarbon radical having from 2 to 4 carbon atoms, such as ethyl, n-propyl, isopropyl, n-butyl, s-butyl, t-butyl and the like.

The term ‘C₂₋₆alkyl’ as used herein as a group or part of a group represents a straight or branched chain fully saturated hydrocarbon radical having from 2 to 6 carbon atoms, such as ethyl, n-propyl, isopropyl, n-butyl, s-butyl, t-butyl, n-pentyl, n-hexyl and the like.

The term ‘C₃₋₆cycloalkyl’ as used herein as a group or part of a group defines a fully saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

Het¹ may be attached to the remainder of the molecule of Formula (I) through any available ring carbon or nitrogen atom as appropriate, if not otherwise specified.

Non-limiting examples of 4- to 7-membered monocyclic fully saturated heterocyclyl containing one or two heteroatoms each independently selected from O, S, and N, include, but are not limited to tetrahydropyranyl, tetrahydrofuranyl, morpholinyl, 1,4-dioxanyl, oxetanyl, pyrrolidinyl, piperidinyl, piperazinyl, and azetidinyl.

Het^{a} is attached to the remainder of the molecule of Formula (I) through any available ring carbon (C-linked).

Non-limiting examples of C-linked 4- to 7-membered monocyclic fully saturated heterocyclyl containing one heteroatom selected from O, S, and N, include, but are not limited to C-linked tetrahydropyranyl, C-linked tetrahydrofuranyl, C-linked oxetanyl, and C-linked azetidinyl.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

It will be clear that a Compound of Formula (I) includes Compounds of Formula (I-x) and (I-y) (both directions of X² being

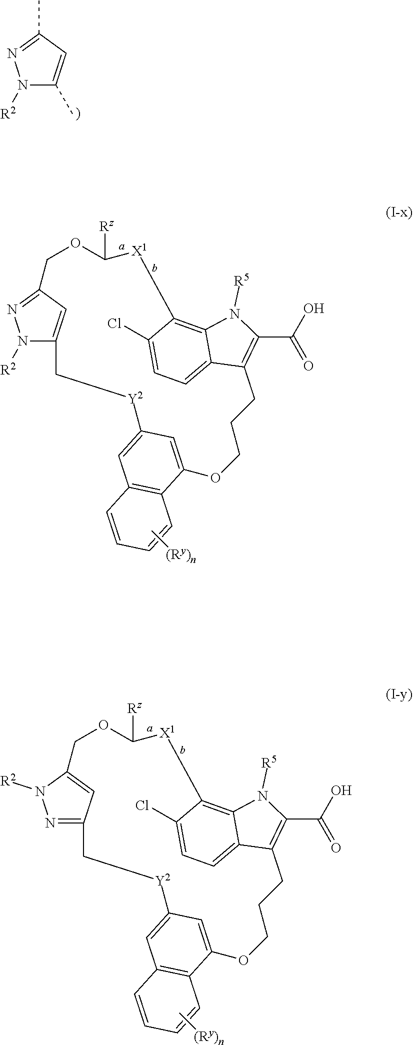

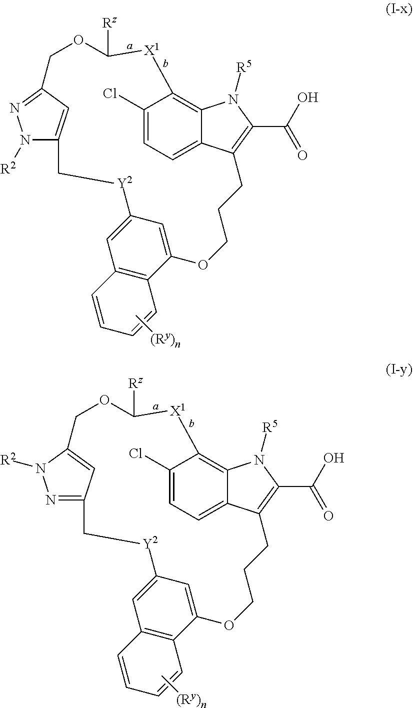

When any variable occurs more than one time in any constituent, each definition is independent.

When any variable occurs more than one time in any formula (e.g. Formula (I)), each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

In particular, the compounds disclosed herein possess axial chirality, by virtue of restricted rotation around a biaryl bond and as such may exist as mixtures of atropisomers. When a compound is a pure atropisomer, the stereochemistry at each chiral center may be specified by either Rₐ or Sₐ. Such designations may also be used for mixtures that are enriched in one atropisomer. Further description of atropisomerism and axial chirality and rules for assignment of configuration can be found in Eliel, E. L. & Wilen, S. H. ‘Stereochemistry of Organic Compounds’ John Wiley and Sons, Inc. 1994.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light. Optically active (Rₐ)- and (Sₐ)-atropisomers may be prepared using chiral synthons, chiral reagents or chiral catalysts, or resolved using conventional techniques well known in the art, such as chiral HPLC.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer; when a compound of Formula (I) is for instance specified as Rₐ, this means that the compound is substantially free of the Sₐ atropisomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S, ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein

X¹ represents

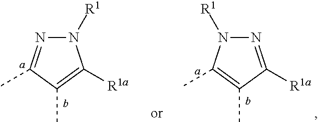

wherein ‘a’ and ‘b’ indicate how variable X¹ is attached to the remainder of the molecule;
R^{y} represents halo;
n represents 0 or 1;
R^{z} represents hydrogen or C₁₋₄alkyl optionally substituted with one Het¹;
Het¹ represents a 4- to 7-membered monocyclic fully saturated heterocyclyl containing one or two heteroatoms each independently selected from O, S, and N;
X² represents

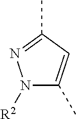

which can be attached to the remainder of the molecule in both directions;
R¹ represents C₁₋₆alkyl optionally substituted with one —OR³ substituent;
R² represents hydrogen; methyl; or C₂₋₆alkyl optionally substituted with one —NR^{4a}R^{4b} substituent;
R^{1a} represents methyl or ethyl;
R³ represents —C₂₋₄alkyl-O—C₁₋₄alkyl;
R^{4a} and R^{4b} are each independently selected from C₁₋₄alkyl;
R⁵ represents methyl;
Y² represents —CH₂— or —S—;
and the pharmaceutically acceptable salts and the solvates thereof.

The present invention relates in particular to compounds of Formula (I) as defined herein, and the tautomers and the stereoisomeric forms thereof, wherein

X¹ represents

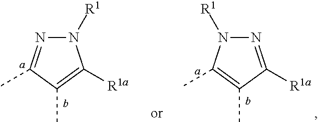

wherein ‘a’ and ‘b’ indicate how variable X¹ is attached to the remainder of the molecule;
R^{y} represents halo;
n represents 0 or 1;
R^{z} represents hydrogen or C₁₋₄alkyl;
X² represents

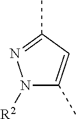

which can be attached to the remainder of the molecule in both directions;
R¹ represents C₁₋₆alkyl optionally substituted with one —OR³ substituent;
R² represents hydrogen; methyl; or C₂₋₆alkyl optionally substituted with one —NR^{4a}R^{4b} substituent;
R^{1a} represents methyl or ethyl;
R³ represents —C₂₋₄alkyl-O—C₁₋₄alkyl;
R^{4a} and R^{4b} are each independently selected from C₁₋₄alkyl;
R⁵ represents methyl;
Y² represents —CH₂— or —S—;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein

Y² represents —S—.

In an embodiment, the present invention relates to those compounds of Formula (I) and the pharmaceutically acceptable salts, and the solvates thereof, or any subgroup thereof as mentioned in any of the other embodiments, wherein

Y² represents —CH₂—.

In an embodiment, the present invention relates to those compounds of Formula (I) and the pharmaceutically acceptable salts, and the solvates thereof, or any subgroup thereof as mentioned in any of the other embodiments, wherein R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 1; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

R¹ represents C₁₋₆alkyl optionally substituted with one —OR³ substituent.

In an embodiment, the present invention relates to those compounds of Formula (I) and the pharmaceutically acceptable salts, and the solvates thereof, or any subgroup thereof as mentioned in any of the other embodiments, wherein R¹ represents methyl.

In an embodiment, the present invention relates to those compounds of Formula (I) and the pharmaceutically acceptable salts, and the solvates thereof, or any subgroup thereof as mentioned in any of the other embodiments, wherein R¹ represents C₁₋₆alkyl optionally substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein

R¹ represents hydrogen; or C₁₋₆alkyl optionally substituted with one substituent selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein

R¹ represents hydrogen; or C₁₋₆alkyl optionally substituted with one or two substituents selected from the group consisting of Het¹, —OR³, and —NR^{4a}R^{4b}.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein R² represents hydrogen.

In an embodiment, the present invention relates to those compounds of Formula (I) and the pharmaceutically acceptable salts, and the solvates thereof, or any subgroup thereof as mentioned in any of the other embodiments, wherein R² represents

C₂₋₆alkyl optionally substituted with one —NR^{4a}R^{4b} substituent.

In an embodiment, the present invention relates to those compounds of Formula (I) and the pharmaceutically acceptable salts, and the solvates thereof, or any subgroup thereof as mentioned in any of the other embodiments, wherein R⁵ represents methyl.

In an embodiment, the present invention relates to those compounds of Formula (I) and the pharmaceutically acceptable salts, and the solvates thereof, or any subgroup thereof as mentioned in any of the other embodiments, wherein R^{z} represents C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein R^{z} represents methyl.

In an embodiment, the present invention relates to those compounds of Formula (I) and the pharmaceutically acceptable salts, and the solvates thereof, or any subgroup thereof as mentioned in any of the other embodiments, wherein

R^{z} represents C₁₋₄alkyl optionally substituted with one Het¹.

In an embodiment, the present invention relates to those compounds of Formula (I) and the pharmaceutically acceptable salts, and the solvates thereof, or any subgroup thereof as mentioned in any of the other embodiments, wherein

R^{z} represents C₁₋₄alkyl optionally substituted with one Het¹; and
Het¹ represents morpholinyl.

In an embodiment, the present invention relates to those compounds of Formula (I) and the pharmaceutically acceptable salts, and the solvates thereof, or any subgroup thereof as mentioned in any of the other embodiments, wherein

R^{z} represents C₁₋₄alkyl optionally substituted with one Het¹; and
Het¹ represents 1-morpholinyl.

In an embodiment, the present invention relates to those compounds of Formula (I) and the pharmaceutically acceptable salts, and the solvates thereof, or any subgroup thereof as mentioned in any of the other embodiments, wherein Het¹ represents morpholinyl.

In an embodiment, the present invention relates to those compounds of Formula (I) and the pharmaceutically acceptable salts, and the solvates thereof, or any subgroup thereof as mentioned in any of the other embodiments, wherein Het¹ represents 1-morpholinyl.

In an embodiment, the present invention relates to those compounds of Formula (I) and the pharmaceutically acceptable salts, and the solvates thereof, or any subgroup thereof as mentioned in any of the other embodiments, wherein n represents 0.

In an embodiment, the present invention relates to those compounds of Formula (I) and the pharmaceutically acceptable salts, and the solvates thereof, or any subgroup thereof as mentioned in any of the other embodiments, wherein n represents 1.

In an embodiment, the present invention relates to those compounds of Formula (I) and the pharmaceutically acceptable salts, and the solvates thereof, or any subgroup thereof as mentioned in any of the other embodiments, wherein n represents 2.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through a nitrogen atom.

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below:

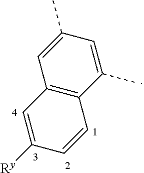

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below; and wherein R^{y} represents fluoro:

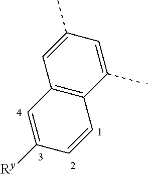

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

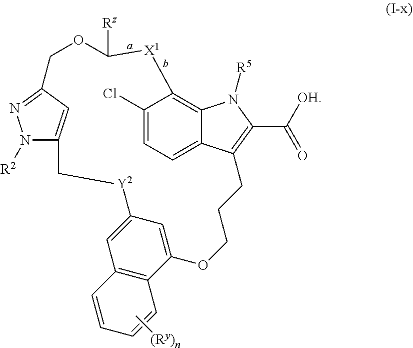

It will be clear that all variables in the structure of Formula (I-x), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

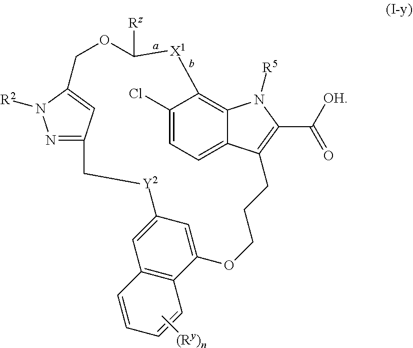

It will be clear that all variables in the structure of Formula (I-y), are defined as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, any pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or can be prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

All variables are as defined for the compound of Formula (I) unless otherwise indicated or if it is clear from the context. The meaning of the chemical abbreviations used in the schemes below are as defined in the Table with abbreviations in the Examples.

Compounds of Formula (I) can be prepared according to Scheme 1,

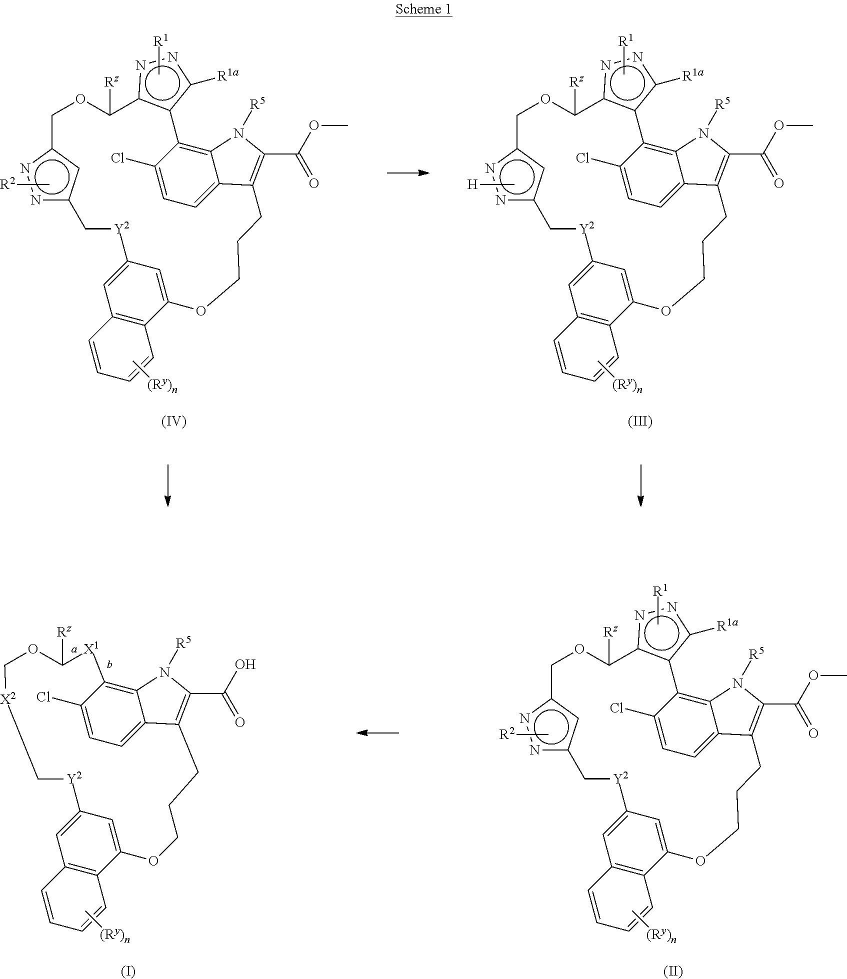

By reacting an intermediate of Formula (II) with a suitable base, such as, for example, LiOH or NaOH, in a suitable solvent, such as water or a mixture of water and a suitable organic solvent such as dioxane or tetrahydrofuran (THF), or a mixture of methanol (MeOH) and THF, at a suitable temperature, such as room temperature or 60° C.
Intermediates of Formula (II) can be prepared by reacting an intermediate of Formula (III) with a suitable alkylating agent R²L (where L is a suitable leaving group) such as, for example, an alkyl halide, in the presence of a suitable base such as, for example, Cs₂CO₃, in a suitable solvent such as, for example, N,N-dimethylformamide (DMF), at a suitable temperature such as, for example, room temperature or 60° C., followed by a suitable separation of the regioisomers such as, for example, a chromatographic separation.
Intermediates of Formula (III) can be prepared by reacting an intermediate of Formula (IV), wherein R² is defined as a suitable protecting group such as, for example, tetrahydropyranyl (THP), with a suitable deprotecting agent such as, for example, HCl, in a suitable solvent such as, for example, MeOH, THF, or a mixture thereof, at a suitable temperature such as, for example, room temperature.
Alternatively, Compounds of Formula (I) can be prepared by reacting an intermediate of Formula (IV), wherein R² is defined as in Formula (I), with a suitable base, such as, for example, LiOH or NaOH, in a suitable solvent, such as water or a mixture of water and a suitable organic solvent such as dioxane or tetrahydrofuran (THF), or a mixture of MeOH and THF, at a suitable temperature, such as room temperature or 60° C.

An intermediate of Formula (II) might have a protecting group in the R¹ position, such as for example tetrahydropyranyl. In such a case, the intermediate of Formula (II) is reacted with a suitable deprotecting agent, such as, for example, pTsOH (p-toluenesulfonic acid) or HCl, in a suitable solvent such as, for example, iPrOH (2-propanol), at a suitable temperature such as, for example, room temperature. In a next step the obtained unprotected intermediate can be reacted with a suitable alkylating agent R¹L (where L is as suitable leaving group) such as, for example, an alkyl halide, in the presence of a suitable base such as, for example, Cs₂CO₃, in a suitable solvent such as, for example, DMF, at a suitable temperature such as, for example, room temperature or 60° C. It will be clear for somebody skilled in the art that, in case R² is a protective group, the protecting group in the R¹ position will have to be an orthogonal protective group to R².

Intermediates of Formula (IV), wherein R^{1a}, R⁵, (R^{y})ₙ, R^{z} and Y² are defined as in Formula (I), and wherein R¹ and R² each independently are as defined in Formula (I) or can be a protective group such as, for example THP or paramethoxybenzyl (PMB), can be prepared according to Scheme 2,

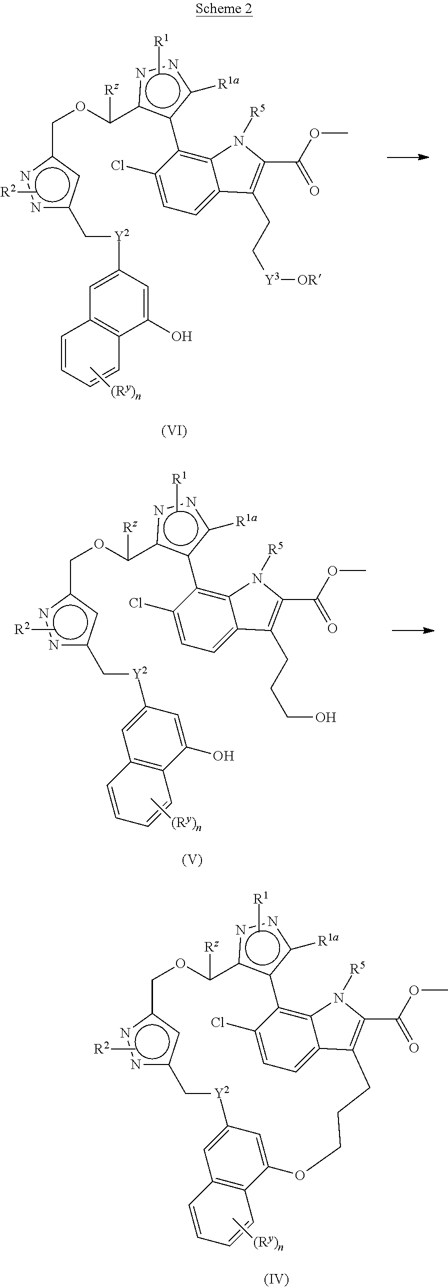

by reacting an intermediate of Formula (V), with a suitable reagent such as, for example, diethyl azodicarboxylate (DEAD) or di-tert-butyl azodicarboxylate (DTBAD), in the presence of a suitable phosphine such as, for example, PPh₃, in a suitable solvent such as, for example, THF, toluene, or a mixture thereof, at a suitable temperature such as, for example, room temperature or 70° C.
Intermediates of Formula (V) can be prepared by reacting an intermediate of Formula (VI), wherein Y³ is C═O and R′ is Me, with a suitable reducing agent such as, for example, BH₃·DMS (borane dimethylsulfide), in a suitable solvent such as, for example, THF, at a suitable temperature such as, for example, room temperature or 50° C.
Alternatively, Intermediates of Formula (V) can be prepared by reacting an intermediate of Formula (VI), wherein Y³ is CH₂ and R′ is a suitable protecting group such as tert-butyldimethylsilyl (TBDMS), with a suitable deprotecting agent such as, for example, tetrabutylammonium fluoride (TBAF), in a suitable solvent such as, for example, THF, at a suitable temperature such as, for example, room temperature.

Intermediates of Formula (VI)), wherein R^{1a}, R⁵, (R^{y})ₙ, R^{z} are defined as in Formula (I), wherein R¹ and R² each independently are as defined in Formula (I) or can be a protective group such as, for example THP or paramethoxybenzyl (PMB), wherein Y³/R′ is C═O/Me or Y³/R′ is CH₂/TBDMS, and Y² is defined as CH₂ can be prepared according to Scheme 3,

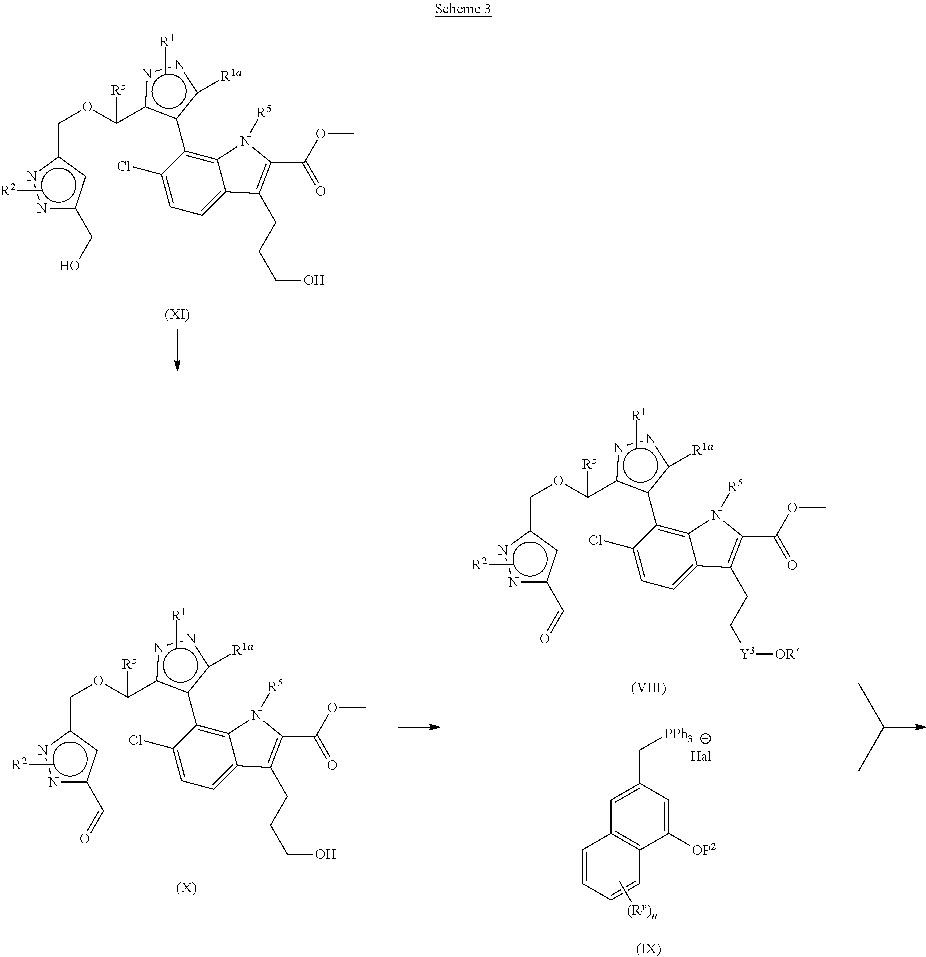

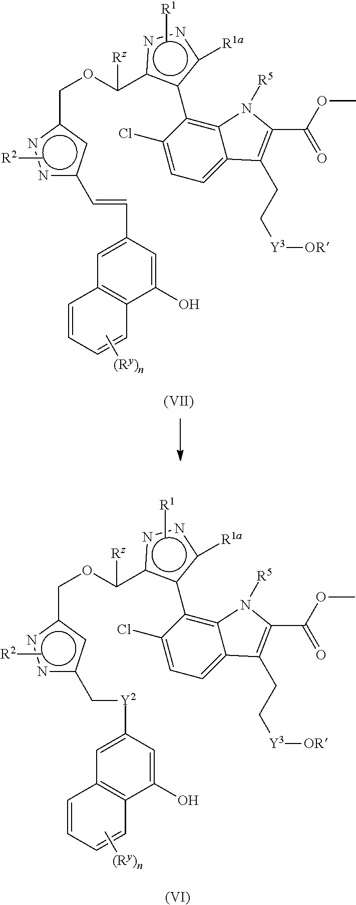

by reacting an intermediate of Formula (VII), with a suitable hydrogenating agent such as, for example, hydrogen gas, at a suitable pressure such as, for example, 3.5 atm, in the presence of a suitable catalyst such as, for example, Pd/C, in a suitable solvent such as, for example, EtOAc, at a suitable temperature such as, for example, room temperature.
Intermediates of Formula (VII) can be prepared by reacting an intermediate of Formula (VIII) with an intermediate of Formula (IX), in the presence of a suitable base such as, for example, NaH, in a suitable solvent such as, for example, THF, at a suitable temperature such as, for example, room temperature.
Intermediates of Formula (VIII) wherein Y³ is CH₂ and R′ is a protecting group such as TBDMS can be prepared by reacting an intermediate of Formula (X) with a suitable protecting group precursor such as, for example, TBDMSCl, in the presence of a suitable base such as, for example, imidazole, in a suitable solvent such as, for example, DCM, at a suitable temperature such as, for example, room temperature.
Intermediates of Formula (X) can be prepared by reacting an intermediate of Formula (XI) with a suitable oxidizing agent such as, for example, MnO₂, in a suitable solvent such as, for example, DCM, at a suitable temperature such as, for example, reflux of the solvent.
It will be clear for a skilled person that, in case R¹ and/or R² are protective groups, the protecting groups P² and R′ will have to be orthogonal protective groups.

Alternatively, intermediates of Formula (VI), wherein Ria, R⁵, (R^{y})ₙ, R^{z} are defined as in Formula (I), wherein R¹ and R² each independently are as defined in Formula (I) or can be a protective group such as, for example THP or paramethoxybenzyl (PMB), wherein Y³/R′ is C═O/Me or Y³/R′ is CH₂/TBDMS, and Y² is defined as CH₂ can be prepared according to Scheme 4,

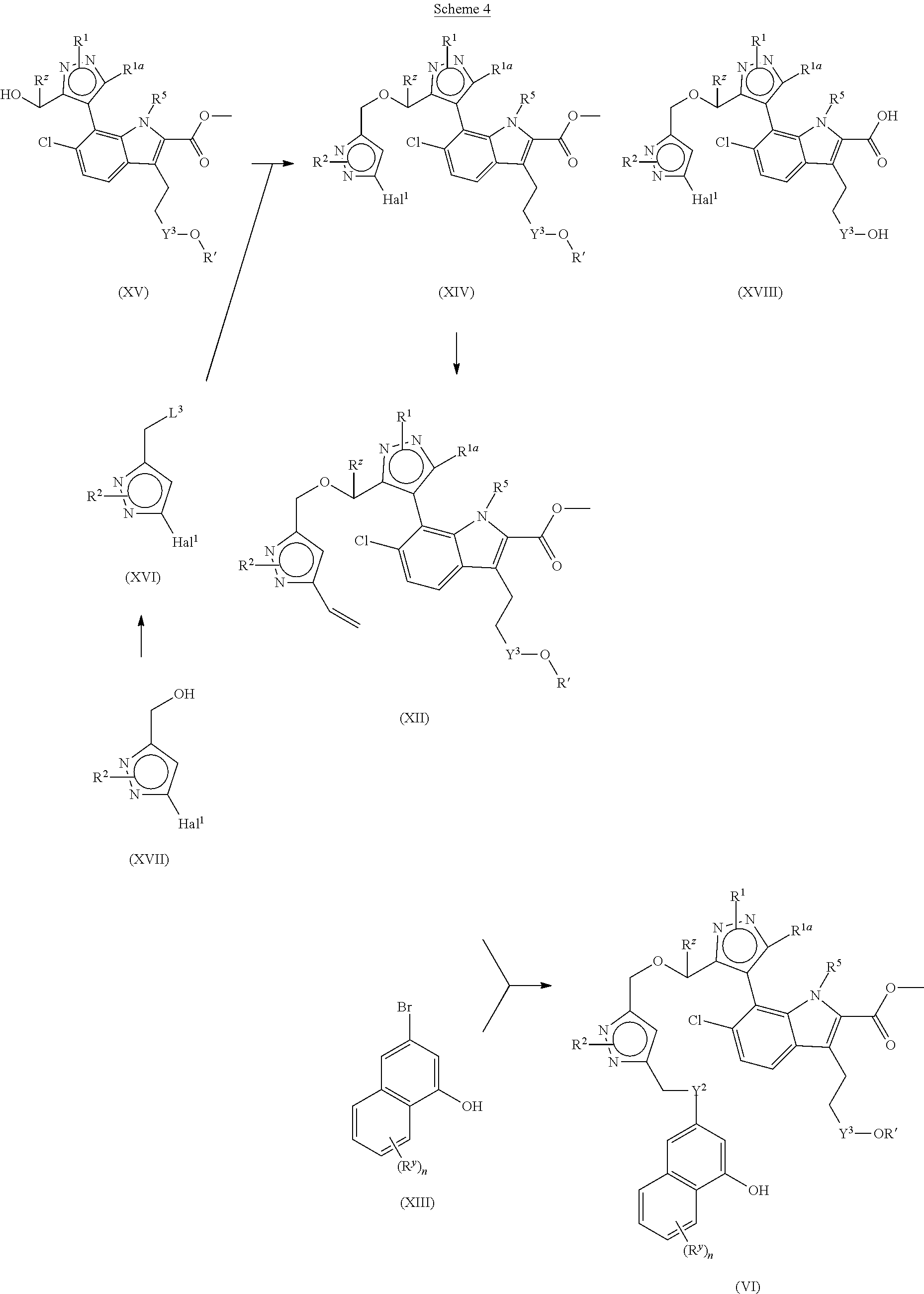

By reacting an intermediate of Formula (XII) with an intermediate of Formula (XIII) in a two-steps procedure, first by reacting an intermediate of Formula (XII) with a suitable borylating agent such as, for example, 9-borabicyclo[3.3.1]nonane (9-BBN) (CAS [280-64-8]), in a suitable solvent such as, for example, tetrahydrofuran (THF), at a suitable temperature such as, for example, room temperature; then by reacting the formed intermediate with an intermediate of Formula (XIII) in the presence of a suitable catalyst such as, for example, 1,1′-bis (di-t-butylphosphino)ferrocene palladium dichloride (PdCl₂(dtbpf)) (CAS [95408-45-0]), in the presence of a suitable base such as, for example, K₃PO₄, in a suitable solvent such as, for example, THF, at a suitable temperature such as, for example, 95° C.
Intermediates of Formula (XII) can be prepared by reacting an intermediate of Formula (XIV) wherein Hal¹ is defined as a suitable halogen such as, for example, Br, with a suitable vinyl derivative such as, for example, vinylboronic acid pinacol ester, in the presence of a suitable catalyst such as, for example, PdCl₂(dtbpf) (CAS [95408-45-0]), in the presence of a suitable base such as, for example, Na₂CO₃, in a suitable solvent such as, for example, a mixture of 1,4-dioxane and water, at a suitable temperature such as, for example, 100° C.
Intermediates of Formula (XIV) can be prepared by reacting an intermediate of Formula (XV) with an intermediate of Formula (XVI) wherein L³ is a suitable leaving group such as, for example I (iodide), in the presence of a suitable base such as, for example, NaH, in a suitable solvent such as, for example, DMF, at a suitable temperature such as, for example, 0° C. A skilled person will recognize that this step might lead to deprotection of ester functions and/or the TBDMS protecting group in case Y³ is CH₂ and R′ is TBDMS, in which case intermediates of Formula (XVIII) or partially protected forms thereof would be obtained. These can be converted to intermediates of Formula (XIV), first by reacting alcohol functions (if present) with a suitable protecting group such as, for example, TBDMSCl, in the presence of a suitable base such as, for example, imidazole or DMAP, or a mixture thereof, in a suitable solvent such as, for example, DCM, at a suitable temperature such as, for example, room temperature, and second by reacting carboxylic acid functions with a suitable methylating agent, such as, for example, methyl iodide, in the presence of a suitable base such as, for example, K₂CO₃, in a suitable solvent such as, for example, DMF, at a suitable temperature such as, for example, room temperature.
Intermediates of Formula (XVI) can be prepared by reacting intermediates of Formula (XVII), first with a suitable activating agent such as, for example, methanesulfonic anhydride, in the presence of a suitable base such as, for example, DIPEA, in a suitable solvent such as, for example, THF, at a suitable temperature such as, for example, room temperature; then by reacting this activated intermediate with a suitable source of L³ such as, for example, KI. It will be clear for a skilled person that, in case R¹ or R² are protective groups, the protecting group R′ will have to be an orthogonal protective group.
Intermediates of Formula (XIII) and (XVII) are commercially available or can be prepared according to reaction protocols known by a skilled person.

Intermediates of Formula (XV), wherein R^{1a} and R⁵ are defined as in Formula (I), R¹ is a protective group such as, for example THP or PMB, or R¹ is defined as in Formula (I), R^{z} is defined as H (hydrogen), and Y³/R′ is C═O/Me or Y³/R′ is CH₂/TBDMS, can be prepared according to Scheme 5,

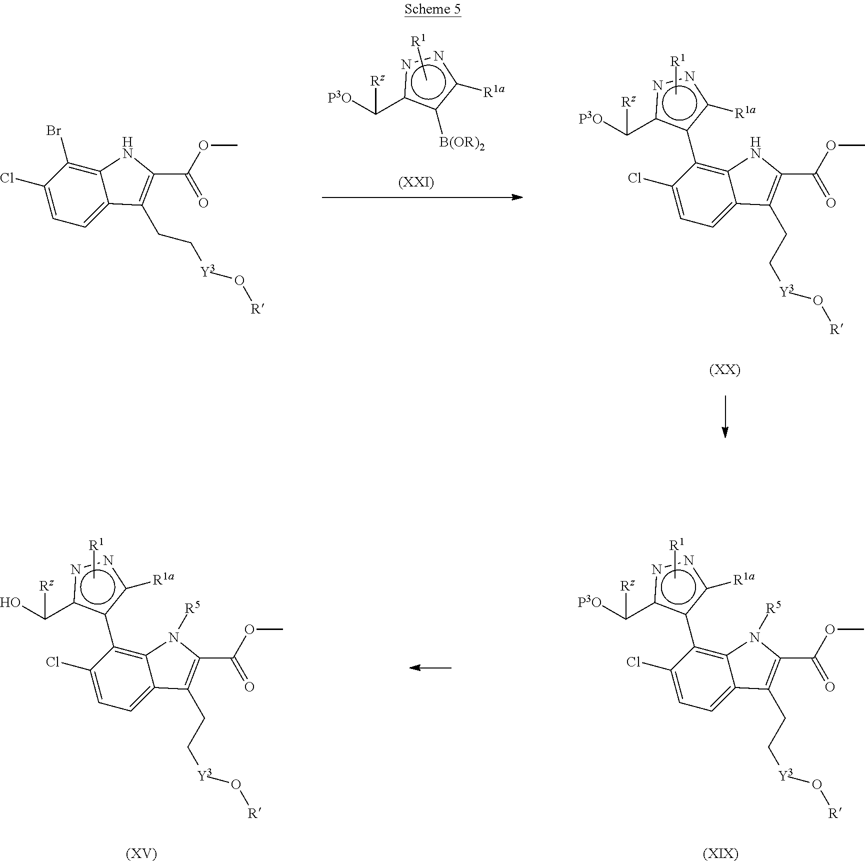

by reacting an intermediate of Formula (XIX), wherein P³ is a suitable protective group such as, for instance, p-methoxybenzyl, with a suitable deprotecting agent such as, for example, DDQ, in a suitable solvent such as, for example, DCM, at a suitable temperature such as, for example, room temperature.
Intermediates of Formula (XIX) can be prepared by reacting an intermediate of Formula (XX) with a suitable alkylating reagent such as, for example, Mel (methyl iodide), in the presence of a suitable base such as, for example, Cs₂CO₃, in a suitable solvent such as, for example, DMF, at a suitable temperature such as, for example, room temperature.
Intermediates of Formula (XX) wherein P³ is a suitable protecting group such as, for example, PMB or THP, Y³ is C═O, and R′ is Me, can be prepared by reacting methyl 7-bromo-6-chloro-3-(3-methoxy-3-oxopropyl)-1H-indole-2-carboxylate (CAS [2143010-85-7]) with an intermediate of Formula (XXI), in the presence of a suitable catalyst such as, for example, [1,1′-bis(di-tert-butylphosphino)ferrocene]dichloropalladium(II) (Pd(dtbpf)Cl₂), in the presence of a suitable base such as, for example, Cs₂CO₃, in a suitable solvent such as, for example, a mixture of THE and water, at a suitable temperature such as, for example, 100° C.
It will be clear for a skilled person that in case R¹ is a protecting group, P³ will have to be an orthogonal protective group to R¹. It will also be clear for a skilled person that in this case additional orthogonality of both the R¹ and P³ protective groups will have to be respected versus R′.
Alternatively, this whole synthetic pathway may start from methyl 7-bromo-6-chloro-3-(3-hydroxypropyl)-1H-indole-2-carboxylate (CAS [2245716-18-9]) after its protection by a suitable protecting group reagent such as, for example, TBDMSCl (tert-butyldimethylchlorosilane), in the presence of a suitable base such as, for example, Et₃N (triethylamine) or DMAP (4-dimethylaminopyridine), or a mixture thereof, in a suitable solvent such as, for example, THF, at a suitable temperature such as, for example, room temperature, leading to intermediates wherein Y³ is CH₂ and R′ is a suitable protecting group such as TBDMS.

Alternatively, intermediates of Formula (XIX) wherein R^{1a} and R⁵ are defined as in Formula (I), wherein R¹ and R² each independently are as defined in Formula (I) or can be a protective group such as, for example THP or paramethoxybenzyl (PMB), wherein R^{z} is defined as H (hydrogen), and Y³/R′ is CH₂/TBDMS can be prepared from intermediates of Formula (XIX) wherein Y³/R′ is C═O/Me, according to Scheme 6,

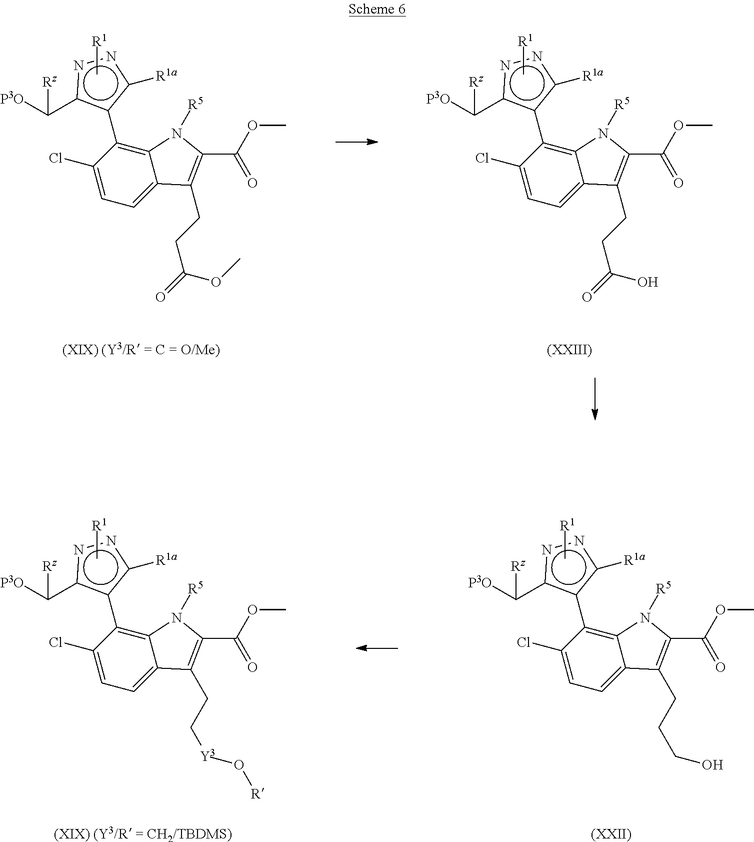

by reacting an intermediate of Formula (XXII) with a suitable protecting group precursor, such as for example, TBDMSCl, in the presence of a suitable base such as, for example, imidazole, in a suitable solvent such as, for example, DCM, at a suitable temperature such as, for example, room temperature.
Intermediates of Formula (XXII) can be prepared by reacting an intermediate of Formula (XXIII) with a suitable reducing agent such as, for example, BH₃·THF complex, in a suitable solvent such as, for example, THF, at a suitable temperature such as, for example, room temperature.
Intermediates of Formula (XXIII) can be prepared by reacting an intermediate of Formula (XIX) wherein Y³/R′ is C═O/Me, with a suitable hydrolysing reagent such as, for example, LiOH, in a suitable solvent such as, for example, a mixture of THE and water, at a suitable temperature such as, for example, room temperature.

Intermediates of Formula (XXI) wherein R^{1a} and R¹ are as defined in Formula (I) or, alternatively, R¹ may also be a suitable protecting group such as, for example, THP; P³ is a suitable protecting group such as, for example, PMB or TBDMS; and B(OR)₂ represents a boronic acid or suitable boronate derivative such as, for example, pinacol ester, can be prepared according to Scheme 7,

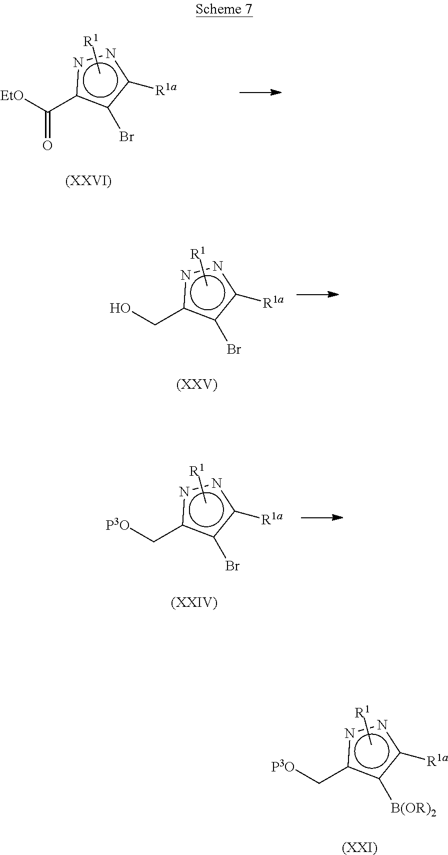

By reacting an intermediate of Formula (XXIV) with a suitable boronate such as, for example, isopropoxyboronic acid pinacol ester, in the presence of a suitable base such as, for example, BuLi, in a suitable solvent, such as, for example, THF, at a suitable temperature such as, for example, −78° C.
Intermediates of Formula (XXIV) can be prepared by reacting an intermediate of Formula (XXV) with a suitable protecting group precursor such as, for example, TBDMSCl, in the presence of a suitable base such as, for example, Et₃N or DMAP, or a mixture thereof, in a suitable solvent such as, for example, THF, at a suitable temperature such as, for example, room temperature.
Intermediates of Formula (XXV) can be prepared by reacting an intermediate of Formula (XXVI) with a suitable reducing agent such as, for example, LiBH₄, in a suitable solvent such as, for example, 2-methyltetrahydrofuran (2-MeTHF), at a suitable temperature such as, for example, room temperature.
Intermediates of Formula (XXVI) can be prepared according to reaction protocols known by a skilled person.

Intermediates of Formula (IX) wherein (R^{y})ₙ is defined as in Formula (I), and P² is a suitable protecting group such as, for example, PMB or tert-butyldiphenylsilyl (TBDPS), can be prepared according to Scheme 8,

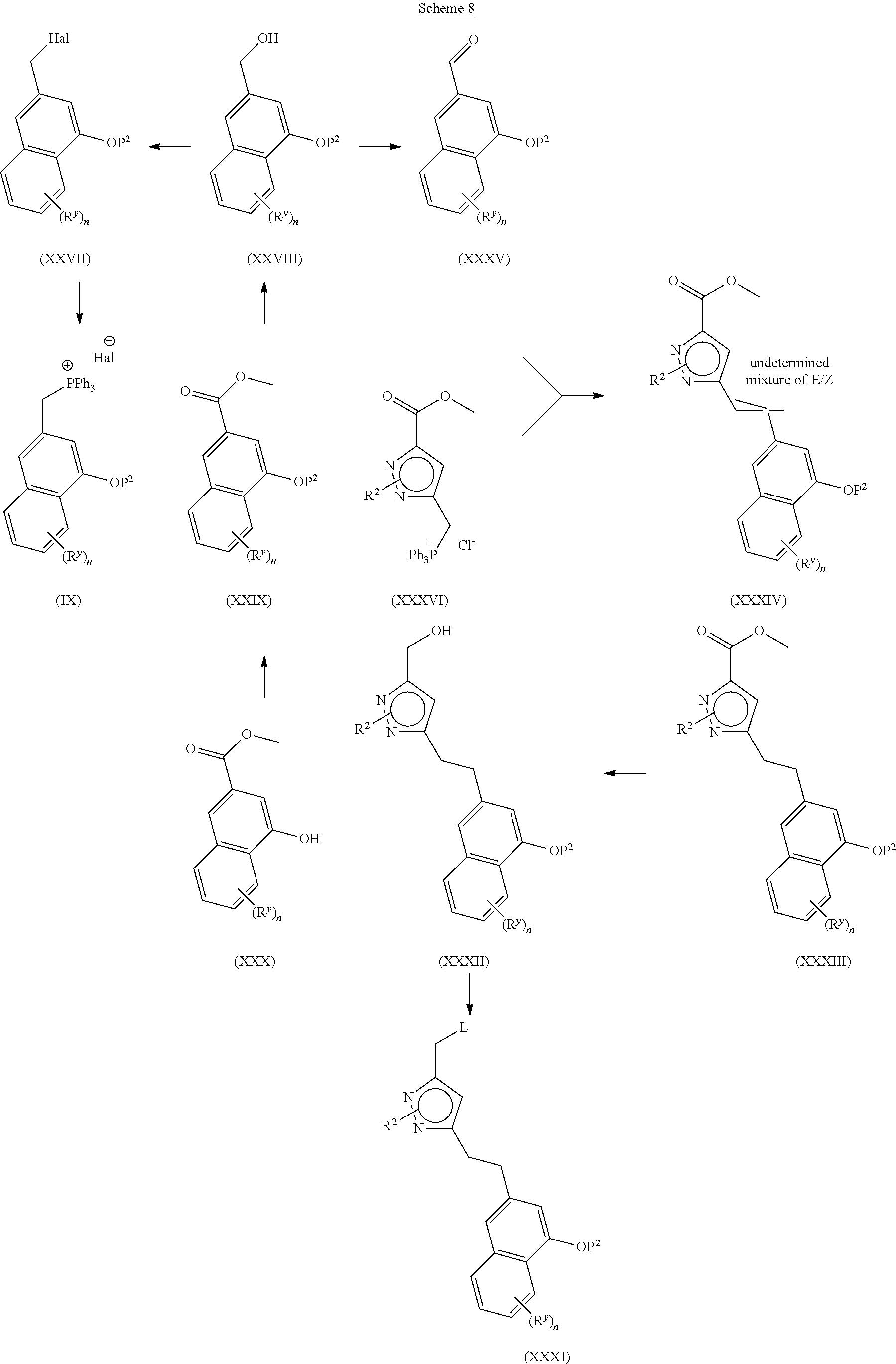

by reacting an intermediate of Formula (XXVII) with a suitable phosphine such as, for example, PPh₃, in a suitable solvent such as, for example, DCM, at a suitable temperature such as, for example, room temperature.
Intermediates of Formula (XXVII), wherein Hal is defined as a suitable halide such as, for example, chloro, can be prepared by first reacting an intermediate of Formula (XXVIII) with a suitable activating agent such as, for example, mesyl anhydride, in the presence of a suitable base such as, for example, DIPEA, followed by addition of a suitable halide source such as, for example, LiCl, in a suitable solvent such as, for example, DCM, at a suitable temperature such as, for example, room temperature.
Intermediates of Formula (XXVIII) can be prepared by reacting an intermediate of Formula (XXIX) with a suitable reducing agent such as, for example, LiAlH₄, in a suitable solvent such as, for example, THF, at a suitable temperature such as, for example, 0° C.
Intermediates of Formula (XXIX) can be prepared by reacting an intermediate of Formula (XXX) with a suitable protecting reagent such as, for example, tert-butyl(chloro)diphenylsilane (TBDPSCl) or 4-methoxybenzyl chloride (PMBCl), in the presence of a suitable base such as, for example, imidazole or NaH, in a suitable solvent such as, for example, DMF, at a suitable temperature such as, for example, room temperature.
Intermediates of Formula (XXX) are commercially available or can be prepared according to reaction protocols known by a skilled person.

Intermediates of Formula (XXXI) wherein (R^{y})ₙ and R² are as defined in Formula (I) or, alternatively, R² may also be a suitable protecting group such as, for example, THP, P² is a suitable protecting group such as, for example, PMB or TBDPS, and L is a suitable leaving group such as, for example, iodide, can be prepared according to Scheme 8,
by reacting an intermediate of Formula (XXXII), first with a suitable activating agent such as, for example, mesyl anhydride, in the presence of a suitable base such as, for example, DIPEA, followed by addition of a suitable leaving group precursor such as, for example, NaI, in a suitable solvent such as, for example, THF, at a suitable temperature such as, for example, room temperature.
Intermediates of Formula (XXXII) can be prepared by reacting an intermediate of Formula (XXXIII) with a suitable reducing agent such as, for example, LiAlH₄, in a suitable solvent such as, for example, THF, at a suitable temperature such as, for example, 0° C.
Intermediates of Formula (XXXIII) can be prepared by reacting an intermediate of Formula (XXXIV) with a suitable hydrogenating reagent such as, for example, hydrogen gas, at a suitable pressure such as, for example, 15 psi, in the presence of a suitable catalyst such as, for example, Pd/C, in a suitable solvent such as, for example, MeOH or THF, or a mixture thereof, at a suitable temperature such as, for example, room temperature.
Intermediates of Formula (XXXIV) can be prepared by reacting an intermediate of Formula (XXXV) with an intermediate of Formula (XXXVI), in the presence of a suitable base such as, for example, NaH, in a suitable solvent such as, for example, THF, at a suitable temperature such as, for example, −30° C.
Intermediates of Formula (XXXV) can be prepared by reacting an intermediate of Formula (XXVIII) with a suitable oxidizing agent such as, for example, MnO₂, in a suitable solvent such as, for example, DCM, at a suitable temperature such as, for example, room temperature.

Alternatively, intermediates of Formula (VI), wherein R^{1a}, R⁵, (R^{y})ₙ, R^{z} are defined as in Formula (I), wherein R¹ and R² each independently are as defined in Formula (I) or can be a protective group such as, for example THP or paramethoxybenzyl (PMB), and wherein Y³/R′ is C═O/Me or Y³/R′ is CH₂/TBDMS, and wherein Y² is defined as S (sulfur), can be prepared according to Scheme 9,

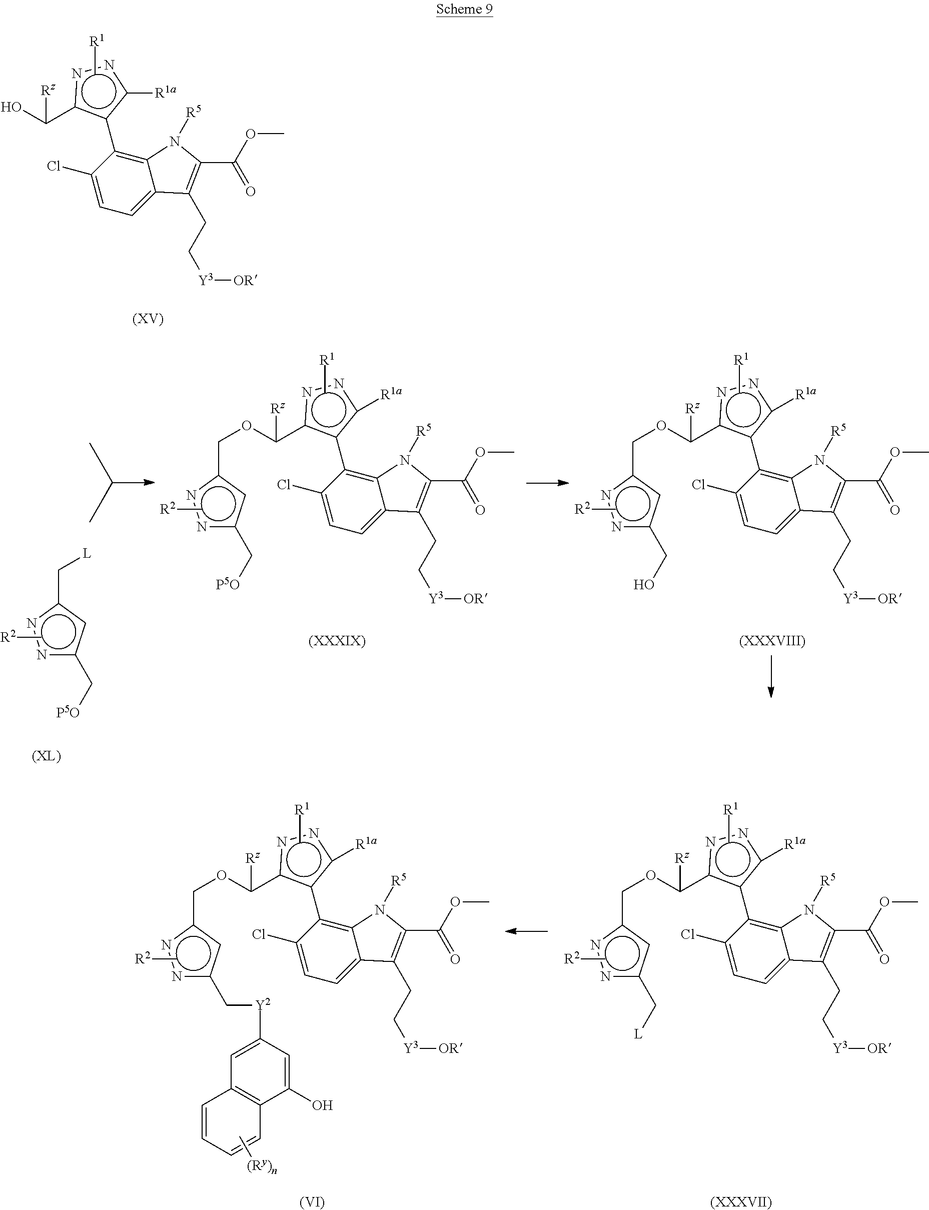

by reacting an intermediate of Formula (XXXVII) with a suitable substituted 3-(acetylthio)naphthalen-1-yl acetate such as, for example, 3-(acetylthio)naphthalen-1-yl acetate, in the presence of a suitable base such as, for example, K₂CO₃, in a suitable solvent such as, for example, MeOH or THF, or a mixture thereof, at a suitable temperature such as, for example, room temperature.
Intermediates of Formula (XXXVII) can be prepared by reacting an intermediate of Formula (XXXVIII) with a suitable activating agent such as, for example, mesyl chloride (MsCl), in the presence of a suitable base such as, for example, Et₃N, in a suitable solvent such as, for example, DCM, at a suitable temperature such as, for example, room temperature.
Intermediates of Formula (XXXVIII) can be prepared by reacting an intermediate of Formula (XXXIX) with a suitable deprotecting agent such as, for example, MgBr₂, in a suitable solvent such as, for example, Et₂O, at a suitable temperature such as, for example, room temperature.
Intermediates of Formula (XXXIX) can be prepared by reacting an intermediate of Formula (XV) with an intermediate of Formula (XL), wherein R² is defined as in Formula (I) or R² is a protective group, P⁵ is a suitable protective group such as, for example, THP or TBDMS, and L is a suitable leaving group such as, for example, I (iodide), in the presence of a suitable base such as, for example, NaH, in a suitable solvent such as, for example, DMF, at a suitable temperature such as, for example, 0° C. or room temperature. It will be clear for a skilled person that, in case R¹ or R² are protective groups, the protective groups P⁵ and R′ will have to be orthogonal protective groups.

Intermediates of Formula (XL), wherein R² is defined as in Formula (I), or R² is a protective group such as, for example THP or PMB, P⁵ is a suitable protective group such as, for example, THP or TBDMS, and L is a suitable leaving group such as, for example, I (iodide), can be prepared according to Scheme 10,

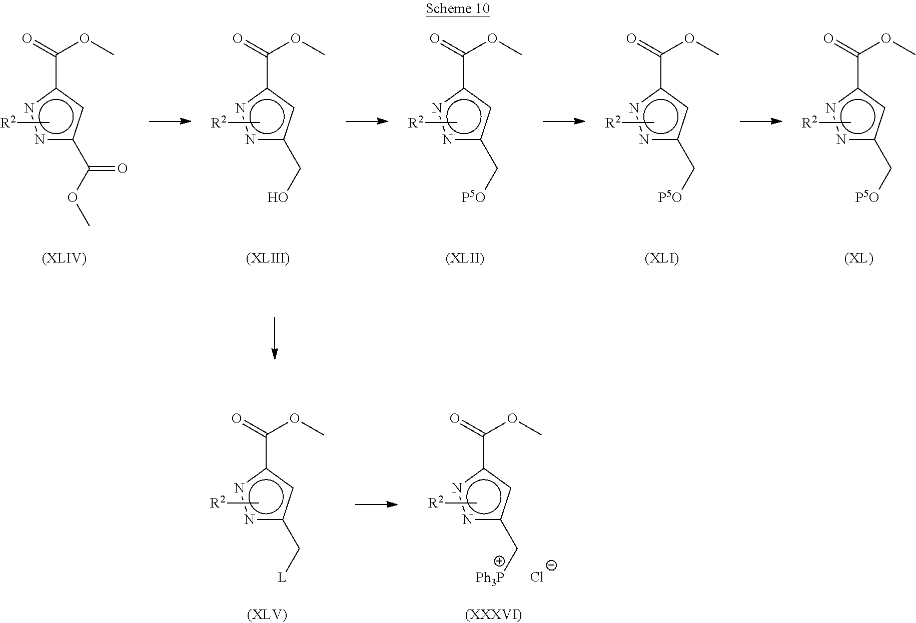

by reacting an intermediate of Formula (XLI) with a suitable activating agent such as, for example, mesyl chloride (MsCl), in the presence of a suitable base such as, for example, Et₃N, followed by addition of a suitable leaving group precursor such as, for example, NaI, in a suitable solvent such as, for example, THF, at a suitable temperature such as, for example, room temperature.
Intermediates of Formula (XLI) can be prepared by reacting an intermediate of Formula (XLII) with a suitable reducing agent such as, for example, LiAlH₄, in a suitable solvent such as, for example, THF, at a suitable temperature such as, for example, 0° C.
Intermediates of Formula (XLII) can be prepared by reacting an intermediate of Formula (XLIII) with a suitable protecting group precursor such as, for example, TBDMSCl, in the presence of a suitable base such as, for example, imidazole, in a suitable solvent such as, for example, DCM, at a suitable temperature such as, for example, 0° C. or room temperature.
Intermediates of Formula (XLIII) can be prepared by reacting an intermediate of Formula (XLIV) with a suitable reducing agent such as, for example, NaBH₄, in a suitable solvent such as, for example, MeOH, Me-THF, or a mixture thereof, at a suitable temperature such as, for example, 0° C. or room temperature.
Intermediates of Formula (XLIV) are commercially available or can be prepared according to reaction protocols known by a skilled person.
It will be clear for a skilled person that, in case R² is a protective group, the protective groups P⁵ will have to be an orthogonal protective group.

Intermediates of Formula (XXXVI), wherein R² is defined as in Formula (I), or R² is a protective group such as, for example THP or PMB, can be prepared according to Scheme 10,
by reacting an intermediate of Formula (XLV) with a suitable phosphine such as, for example, PPh₃, in a suitable solvent such as, for example, ACN, at a suitable temperature such as, for example, at reflux of the solvent.
Intermediates of Formula (XLV) can be prepared by reacting an intermediate of Formula (XLIII) with a suitable activating agent such as, for example, mesyl chloride (MsCl), in a suitable solvent such as, for example, DCM, at a suitable temperature such as, for example, room temperature.

Alternatively, intermediates of Formula (II), wherein R^{1a}, R⁵, (R^{y})ₙ, R^{z} are defined as in Formula (I), wherein R¹ and R² each independently are as defined in Formula (I) or can be a protective group such as, for example THP or paramethoxybenzyl (PMB), and Y² is defined as S (sulphur) can be prepared according to Scheme 11,

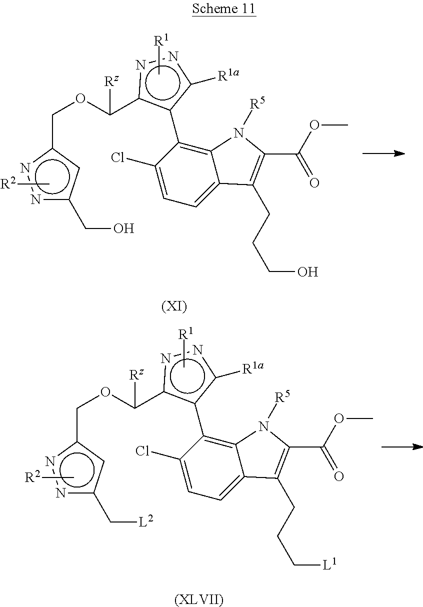

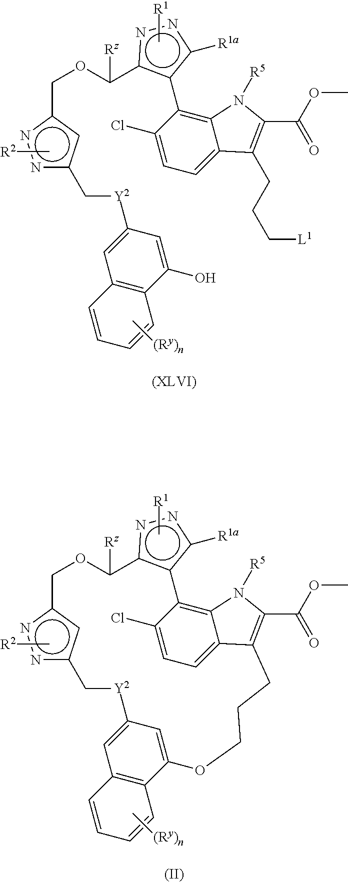

by reacting an intermediate of Formula (XLVI), wherein L¹ is a suitable leaving group such as, for example, mesylate, with a suitable base such as, for example, K₂CO₃, in a suitable solvent such as, for example, ACN, at a suitable temperature such as, for example, at reflux of the solvent.
Intermediates of Formula (XLVI) can be prepared by reacting an intermediate of Formula (XLVII), wherein L² is a suitable leaving group such as, for example, mesylate, with a suitable substituted 3-(acetylthio)naphthalen-1-yl acetate such as, for example, 3-(acetylthio)naphthalen-1-yl acetate, in the presence of a suitable catalyst such as, for example, PPh₃, in the presence of a suitable base such as, for example, K₂CO₃, in a suitable solvent such as, for example, MeOH, at a suitable temperature such as, for example, room temperature.
Intermediates of Formula (XLVII) can be prepared by reacting an intermediate of Formula (XI) with a suitable activating agent such as mesyl chloride (MsCl), in the presence of a suitable base such as, for example, Et₃N, in a suitable solvent such as, for example, THF, at a suitable temperature such as, for example, room temperature.

Intermediates of Formula (XI), wherein R^{1a}, R⁵, R^{z} are defined as in Formula (I), wherein R¹ and R² each independently are as defined in Formula (I) or can be a protective group such as, for example THP or paramethoxybenzyl (PMB), can be prepared according to Scheme 12,

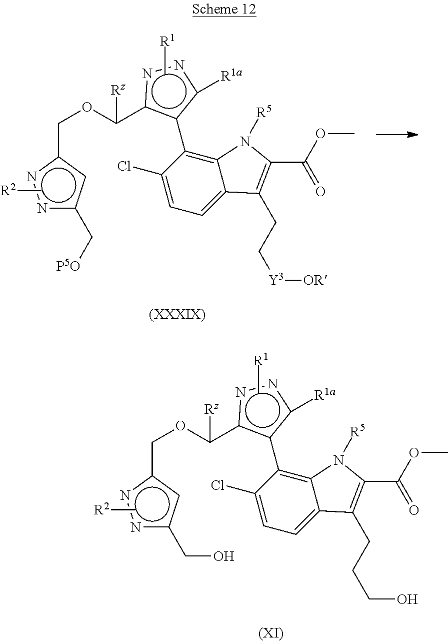

by reacting an intermediate of Formula (XXXIX) with a suitable deprotecting agent such as, for example, MgBr₂ or pTsOH, or HCl, in a suitable solvent such as, for example, Et₂O or 1,4-dioxane, at a suitable temperature such as, for example, room temperature.

Intermediates of Formula (XV), wherein R^{1a} and R⁵ are defined as in Formula (I), R¹ is a protective group such as, for example THP or PMB, or R¹ is defined as in Formula (I), R^{z} is defined as a suitable alkyl group such as, for example, methyl, and Y³/R′ is CH₂/TBDMS, can be prepared according to Scheme 13,

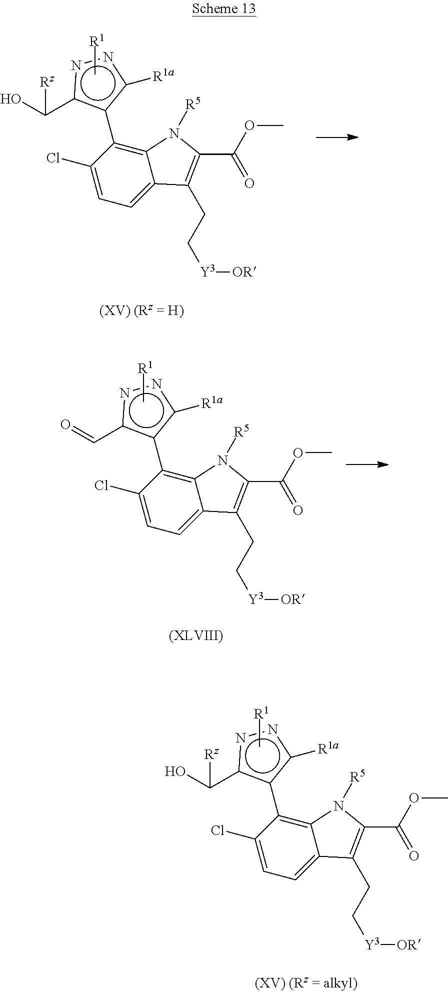

by reacting an intermediate of Formula (XLVIII) with a suitable carbon nucleophile such as, for example, methylmagnesium bromide, in a suitable solvent such as, for example, THF, at a suitable temperature such as, for example, 0° C.
Intermediates of Formula (XLVIII) can be prepared by reacting an intermediate of Formula (XV) wherein R^{z} is defined as H (hydrogen), with a suitable oxidizing agent such as, for example, Dess-Martin periodinane (CAS [87413-09-0]), in a suitable solvent such as, for example, DCM, at a suitable temperature such as, for example, room temperature.

Alternatively, intermediates of Formula (V) wherein R^{1a}, R⁵, R^{z} and (R^{y})ₙ are defined as in Formula (I), wherein R¹ and R² each independently are as defined in Formula (I) or can be a protective group such as, for example THP or paramethoxybenzyl (PMB), and wherein Y² is CH₂, can be prepared according to Scheme 14,

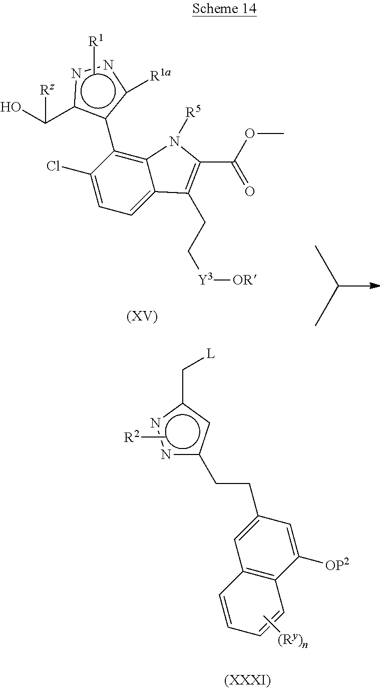

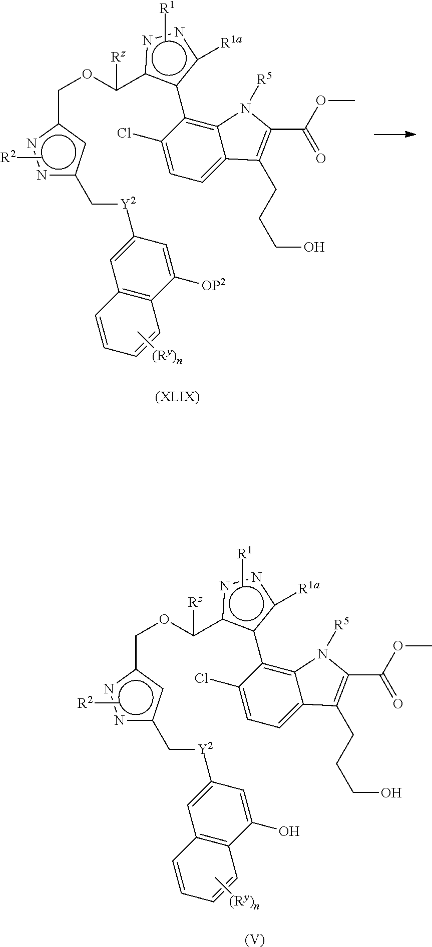

by reacting an intermediate of Formula (XLIX) with a suitable deprotecting agent such as, for example, TBAF, in a suitable solvent such as, for example, THF, at a suitable temperature such as, for example, 0° C.
It will be clear for a skilled person that, in case R¹ and/or R² is a protective group, the protective groups P² will have to be an orthogonal protective group.
Intermediates of Formula (XLIX) can be prepared by reacting an intermediate of Formula (XV) with an intermediate of Formula (XXXI) in the presence of a suitable base such as, for example, NaH, in a suitable solvent such as, for example, THF, at a suitable temperature such as, for example, room temperature.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

### Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8: Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### EXAMPLES

Several methods for preparing the Compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| 9-BNN | 9-borabicyclo[3.3.1]nonane |
| ACN | acetonitrile |
| AcOH | acetic acid |
| Ac₂O | acetic anhydride |
| BuLi | n-butyllithium |
| Celite ® | diatomaceous earth |
| Co | Compound |
| Co. No. | Compound Number |
| DCM | dichloromethane |
| DIBAL | di-isobutylaluminiumhydride |
| DDQ | 2,3-Dichloro-5,6-dicyano-1,4-benzoquinone |
| DEA | diethylamine |
| DIPE | di-isopropylether |
| DIPEA | N,N-diisopropylethylamine |
| DMAP | 4-dimethylaminopyridine |
| DMF | N,N-dimethylformamide |
| DSC | Differential scanning calorimetry |
| DTBAD | Di-tert-butyl Azodicarboxylate |
| eq. | equivalent(s) |
| Et₂O | diethyl ether |
| Et₃N or TEA | trietylamine |
| Et₃N•(HF)₃ | triethylamine trihydrofluoride |
| EtOAc | ethyl acetate |
| EtOH | ethanol |
| h | hour(s) |
| HPLC | high performance liquid chromatography |
| IPA or iPrNH₂ | isopropylamine |
| iPrOH | isopropanol |
| Me | methyl |
| MeI | methyl iodide |
| MeOH | methanol |
| Me-THF or | 2-methyltetrahydrofuran |
| 2-Me-THF | |
| min | minute(s) |
| MP | melting point |
| MsCl | methanesulfonyl chloride |
| Ms₂O | methanesulfonic anhydride |
| Pd/C | palladium on carbon |
| Pd(dtbpf)Cl₂ or | 1,1′-bis (di-t-butylphosphino)ferrocene palladium |
| PdCl₂(dtbpf) | dichloride |
| PPh₃ | triphenylphosphine |
| PTSA | p-toluenesulfonic acid |
| quant. | quantitative |
| rac | racemic |
| Rochelle salt | potassium sodium tartrate tetrahydrate |
| RP | reversed phase |
| SFC | supercritical fluid chromatography |
| TBAF | tetrabutylammonium fluoride |
| TBDMSCl | tert-butyldimethylsilyl chloride |
| TBDPSCl | tert-butyl(chloro)diphenylsilane |
| THF | tetrahydrofuran |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

Some intermediates are reported as mixture of regioisomers, e.g. intermediate 53:

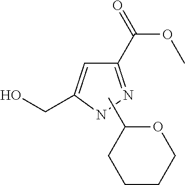

which means the intermediate is a mixture of the 2 regioisomers below:

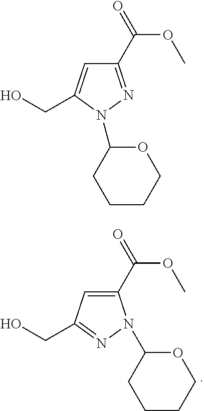

### PREPARATION OF INTERMEDIATES

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Intermediate 1

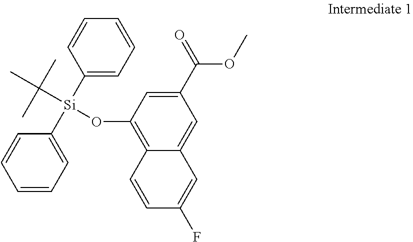 [SMILES: COC(=O)c1cc(O[Si](c2ccccc2)(c2ccccc2)C(C)(C)C)c2ccc(F)cc2c1]

TBDPSCl (14.66 g, 1.5 eq.) was added to a solution of methyl 7-fluoro-4-hydroxy-2-naphthoate (CAS [2092726-85-5]) (8 g, 35.56 mmol) and imidazole (7.26, 3 eq.) in DCM (500 mL), cooled to 0° C. under nitrogen atmosphere. Once the addition was complete, the reaction was stirred at room temperature overnight. The reaction was quenched by addition of water (100 mL). The mixture was extracted with EtOAc (3×200 mL). The combined organic layer was dried over Na₂SO₄, filtered, and concentrated to afford a yellow oil. This oil was purified by flash column chromatography on silica gel (petroleum ether:EtOAc—1:0 to 1:1) to afford Intermediate 1 (14 g, yield: 86%) as a yellow oils.

### Intermediate 2

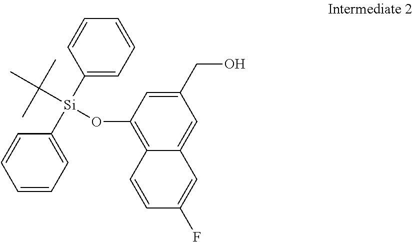 [SMILES: CC(C)(C)[Si](Oc1cc(CO)cc2cc(F)ccc12)(c1ccccc1)c1ccccc1]

LiAlH₄ (1.39 g, 1.2 eq.) was added slowly to a solution of Intermediate 1 (14 g, 30.53 mmol) in THF (200 mL), cooled to 0° C. under nitrogen atmosphere. Once the addition was complete the reaction mixture was stirred at 0° C. for 2 h. The reaction was quenched by slow addition of water (2 mL) followed by a 10% aqueous NaOH solution (2 mL) at 0° C. The heterogeneous mixture was filtered, and the filter cake was washed with DCM (200 mL). The filtrate was evaporated and the residue was purified by flash column chromatography on silica gel (petroleum ether:EtOAc—1:0 to 1:1) to give Intermediate 2 (12 g, yield: 90%) as a yellow solid.

### Intermediate 3

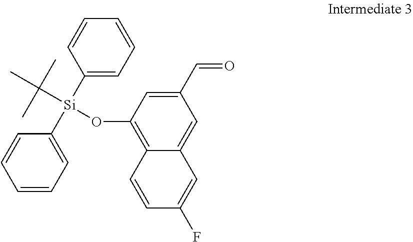 [SMILES: CC(C)(C)[Si](Oc1cc(C=O)cc2cc(F)ccc12)(c1ccccc1)c1ccccc1]

MnO₂ (29 g, 12 eq.) was added to a solution of Intermediate 2 (12 g, 27.87 mmol) in DCM (200 mL) at room temperature. The resulting solution was stirred at room temperature overnight. The reaction mixture was filtered and the filtrate was concentrated. The residue was purified by flash column chromatography over silica gel (eluent: petroleum ether/EtOAc, 100/0 to 50/50) to afford Intermediate 3 (12 g, yield: 99%) as a yellow oil.

### Intermediate 4

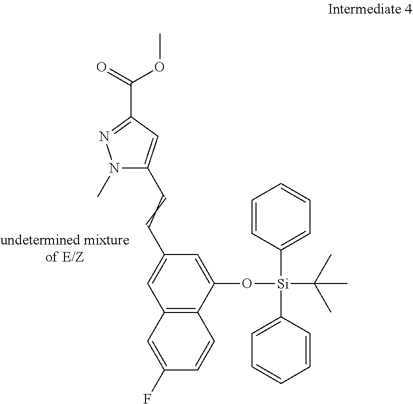 [SMILES: COC(=O)c1cc(C=Cc2cc(O[Si](c3ccccc3)(c3ccccc3)C(C)(C)C)c3ccc(F)cc3c2)n(C)n1]

NaH (60% in mineral oil, 1.45 g, 1.3 eq.) was added to a suspension of ((3-(methoxycarbonyl)-1-methyl-1H-pyrazol-5-yl)methyl)triphenylphosphonium chloride (CAS [2245716-31-6], 13.812 g, 1.1 eq.) in THE (200 mL) at 0° C. The resulting solution was stirred at this temperature for 1 h before being cooled to −30° C. Intermediate 3 (12 g, 27.85 mmol) was added slowly to the solution while maintaining the temperature between −20° C. and −30° C. Once the addition was complete, the reaction was stirred at −30° C. for 2 h. The reaction was quenched by slow addition of water (100 mL). The mixture was extracted with DCM (3×300 mL). The combined organic layer was dried over Na₂SO₄, filtered, and concentrated under reduced pressure. The crude product was purified by column chromatography on silica gel (petroleum ether:EtOAc—1:0 to 1:1) to afford Intermediate 4 (13 g, yield: 82%) as a white solid.

### Intermediate 5

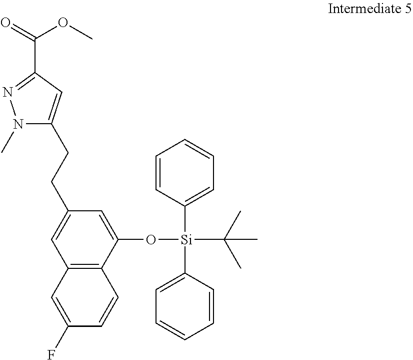 [SMILES: COC(=O)c1cc(CCc2cc(O[Si](c3ccccc3)(c3ccccc3)C(C)(C)C)c3ccc(F)cc3c2)n(C)n1]

A solution of Intermediate 4 (13 g, 23.02 mmol) in MeOH (75 mL) and THF (75 mL) was hydrogenated at 25° C. (15 psi H₂) in the presence of Pd/C (2 g). The reaction mixture was stirred for 16 h. After uptake of H₂ (1 eq.), the catalyst was filtered off and the filtrate was evaporated to afford Intermediate 5 (13 g, yield: 100%) as a colorless oil.

### Intermediate 6

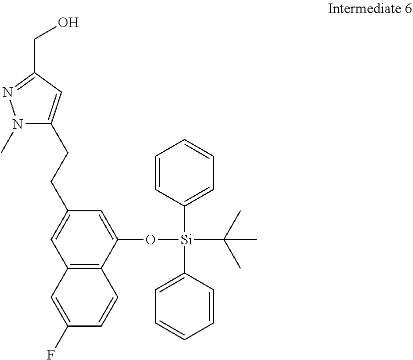 [SMILES: Cn1nc(CO)cc1CCc1cc(O[Si](c2ccccc2)(c2ccccc2)C(C)(C)C)c2ccc(F)cc2c1]

LiAlH₄ (1.05 g, 1.2 eq.) was added portionwise to a solution of Intermediate 5 (13 g, 22.94 mmol) in THE (200 mL) at 0° C., under nitrogen atmosphere. The reaction mixture was stirred at 0° C. for 2 h. Water (1 mL) was then added dropwise, followed by a 10% aqueous NaOH solution (1 mL), at 0° C. The reaction mixture was filtered, the filter cake was washed with DCM (200 mL), and the filtrate was evaporated. The crude product was purified by flash column chromatography over silica gel (eluent: petroleum ether/EtOAc, 100/0 to 0/100) to afford Intermediate 6 (10.4 g, yield: 84%) as a white solid.

### Intermediate 7

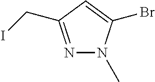

DIPEA (21 mL, 1.98 eq.) and methanesulfonic anhydride (21.45 g, 2 eq.) were added to a solution of (5-bromo-1-methyl-1H-pyrazol-3-yl)methanol (CAS [1782396-26-2], 11.76 g, 0.062 mol) in THE (800 mL) at 0° C. Once the addition was complete, the solution was stirred at room temperature for 1 h before sodium iodide (46.14 g, 5 eq.) was added. Stirring was continued at room temperature for a further 14 h. The reaction mixture was diluted with EtOAc (400 mL), washed with water (250 mL), and the layers were separated. The aqueous phase was extracted with EtOAc (3×100 mL) and the combined organic layer was washed with brine, dried over MgSO₄, and evaporated. The crude product was purified using flash column chromatography (SiO₂, 330 g column, 0-100% EtOAc in heptane) to afford Intermediate 7 (13.00 g, yield: 70%) as a light orange powder.

### Intermediate 8

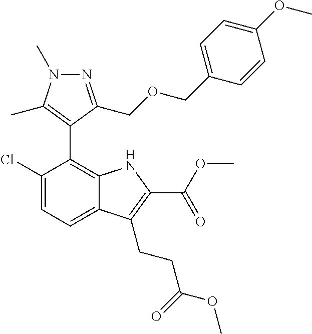

Methyl 7-bromo-6-chloro-3-(3-methoxy-3-oxopropyl)-1H-indole-2-carboxylate (CAS [2143010-85-7]) (2.42 g, 6.19 mmol), 3-(((4-methoxybenzyl)oxy)methyl)-1,5-dimethyl-4-(4,4,5,5-tetramethyl-1,3,2-dioxaborolan-2-yl)-1H-pyrazole (CAS [2143010-90-4]) (3.2 g, 1.15 eq.), and Cs₂CO₃ (4.03 g, 2 eq.) in 1,4-dioxane (50 mL) and water (12 mL) were degassed under nitrogen for 10 min. PdCl₂(dtbpf) (CAS [95408-45-0]) (121 mg, 0.03 eq.) was then added and the reaction mixture was heated at 100° C. overnight. Water and EtOAc were added to the reaction mixture. The layers were separated and the aqueous layer was extracted with EtOAc. The combined organic layer was dried over MgSO₄, filtered, and concentrated. The residue was purified by flash chromatography (silica; heptane/EtOAc gradient) to afford Intermediate 8 (2.35 g, yield: 70%) as a light brown foam.

### Intermediate 9

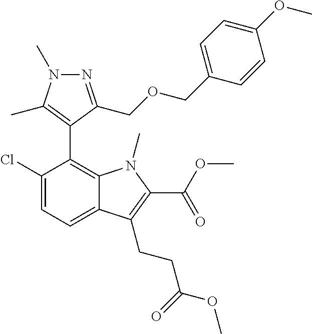

Intermediate 8 (2.35 g, 4.352 mmol) was dissolved in dry DMF (40 mL), and Cs₂CO₃ (2.13 g, 1.5 eq.) was added. The reaction mixture was stirred for 20 min before the addition of iodomethane (542 μL, 2 eq.) at 0° C. The reaction mixture was stirred for 2 h. Water was added and the layers were separated. The aqueous layer was extracted with EtOAc. The combined organic layer was concentrated to afford Intermediate 9 (1.975 g, yield: 79%) as a brown oil, used without purification.

### Intermediate 10

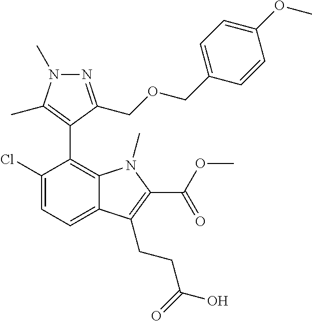

LiOH (3.89 g, 2 eq.) was added a solution of Intermediate 9 (45 g, 81.22 mmol) dissolved in water (300 mL) and THF (100 mL). The reaction mixture was stirred at room temperature for 4 h. The reaction mixture was extracted with DCM (3×500 mL). The organic layer was dried with Na₂SO₄, and the solvent was evaporated to give Intermediate 10 (40 g, yield: 91%) as a yellow oil, used without further purification.

### Intermediate 11

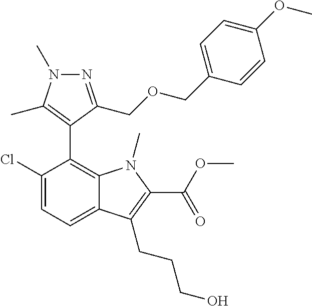

Borane-THF complex (1 M, 296 mL, 2 eq.) was added dropwise to a solution of Intermediate 10 (80 g, 148 mmol) in THE (600 mL) at 0° C. under nitrogen atmosphere. The reaction mixture was stirred for at room temperature for 3 h. The mixture was extracted with DCM (3×500 mL). The combined organic layer was dried with Na₂SO₄, and the solvent was evaporated. The residue was purified by column chromatography on silica gel (eluent: petroleum ether/EtOAc, 100/0 to 1/1). The pure fractions were collected and the solvent was evaporated to give Intermediate 11 (50.83 g, yield: 62%) as a white solid.

### Intermediate 12

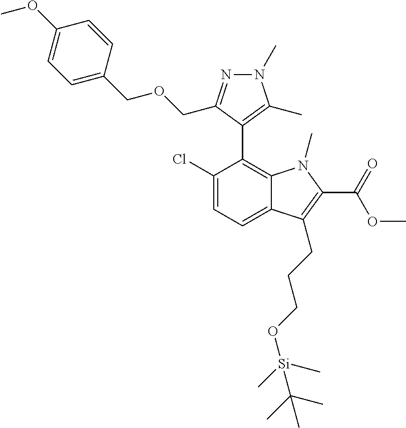

TBDMSCl (4.09 g, 1.5 eq.) was added to a solution of Intermediate 11 (10 g, 18.1 mmol) and imidazole (2.46 g, 2 eq.) stirring in DCM (100 mL) at 0° C. The reaction mixture then stirred at room temperature overnight. The reaction mixture was diluted with DCM (50 mL) and water (100 mL). The organic layer was separated and the aqueous one was extracted with DCM (100 mL). The combined organic layer was dried over MgSO₄, filtered, and concentrated. The crude product was purified by flash column chromatography over silica gel (120 g, gradient: from heptane 100% to heptane/EtOAc 6/4) to give Intermediate 12 (11.4 g, yield: 98%) as a yellowish paste.

### Intermediate 13

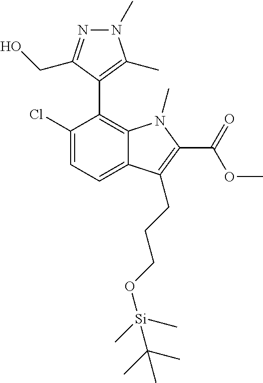 [SMILES: COC(=O)c1c(CCCO[Si](C)(C)C(C)(C)C)c2ccc(Cl)c(-c3c(CO)nn(C)c3C)c2n1C]

DDQ (5.254 g, 1.3 eq.) was added to a solution of Intermediate 12 (11.4 g, 17.804 mmol) in DCM (120 mL) stirring at 0° C. The reaction mixture was stirred at room temperature for 4 h. Celite® was added and, after 5 min of vigorous stirring, the mixture was filtered (washing the Celite® pad with DCM). The filtrate was concentrated and the residue was purified by flash column chromatography on silica gel (120 g, from heptane:EtOAc 100:0 to 0:100) to give Intermediate 13 (5.75 g, yield: 62%)

### Intermediate 14

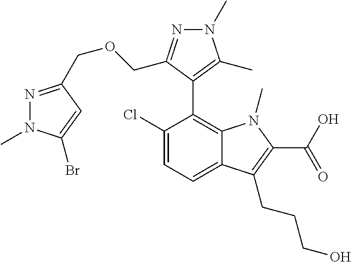 [SMILES: Cc1c(-c2c(Cl)ccc3c(CCCO)c(C(=O)O)n(C)c23)c(COCc2cc(Br)n(C)n2)nn1C]

NaH (60% dispersion in mineral oil, 797 mg, 1.5 eq.) was added to a solution of Intermediate 13 (6.91 g, 13.29 mmol) in anhydrous DMF (100 mL) at 0° C. under nitrogen atmosphere. The reaction mixture was stirred at 0° C. for 10 min before the cooling was removed. A solution of Intermediate 7 (4 g, 1 eq.) in DMF (70 mL) was added via syringe pump (0.2 mL/min). LiOH (1.2 g, 3.7 eq.) was added and the reaction mixture was stirred for 2 h at 30° C. Then, PTSA (16.02 g, 7 eq.) was added and the reaction mixture was stirred at 30° C. for 72 h. The reaction mixture was basified using aqueous NaOH (25% solution) and was then diluted with EtOAc (100 mL). The layers were separated. The aqueous layer was neutralised with AcOH and was extracted with EtOAc (5×100 mL). The combined organic layer was washed with brine, dried over MgSO₄, filtered, and concentrated to afford crude Intermediate 14 (6.2 g) which was used without further purification.

### Intermediate 15

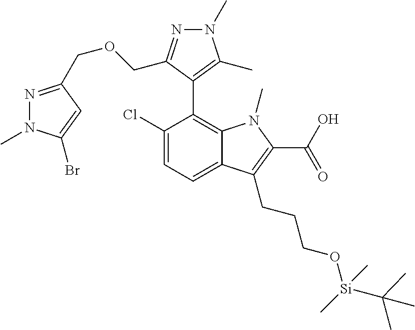 [SMILES: Cc1c(-c2c(Cl)ccc3c(CCCO[Si](C)(C)C(C)(C)C)c(C(=O)O)n(C)c23)c(COCc2cc(Br)n(C)n2)nn1C]

TBDMSCl (2 g, 2.5 eq.) followed by DMAP (0.13 g, 0.2 eq.) was added to a stirred solution of Intermediate 14 (6 g, 5 mmol) and imidazole (1.45 g, 4 eq.) in DCM (50 mL) at room temperature. The reaction mixture was stirred at room temperature for 12 h. More TBDMSCl (2 g, 2.5 eq.) and imidazole (1.45 g, 4 eq.) were added and the reaction mixture was stirred for 1 h. The reaction mixture was diluted with DCM (150 mL) and washed with water (150 mL). The organic layer was dried over MgSO₄, filtered, and concentrated. The residue was purified by flash column chromatography (SiO₂, 80 g column, 0-5% MeOH in DCM) and the pure fractions containing product were combined and concentrated to afford Intermediate 15 (1.7 g, yield: 47%) as a clear yellow oil.

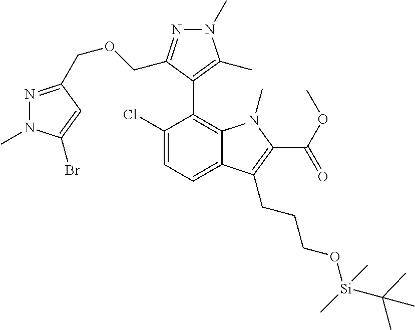 [SMILES: COC(=O)c1c(CCCO[Si](C)(C)C(C)(C)C)c2ccc(Cl)c(-c3c(COCc4cc(Br)n(C)n4)nn(C)c3C)c2n1C]

Mel (390 μL, 2.5 eq.) was added dropwise to a solution of Intermediate 15 (1.7 g, 2.5 mmol) and K₂CO₃ (346 mg, 1 eq.) in anhydrous DMF (9 mL) cooled to 0° C. The reaction was then stirred at room temperature for 10 min. The reaction mixture was diluted with EtOAc (10 mL) and water (50 mL). The aqueous layer was extracted with DCM (5×10 mL). The combined organic layer was washed with water (4×5 mL), dried over MgSO₄, filtered, and concentrated to afford Intermediate 16 (1.61 g, 80% pure, 74%) as a clear yellow oil, used without further purification.

### Intermediate 17

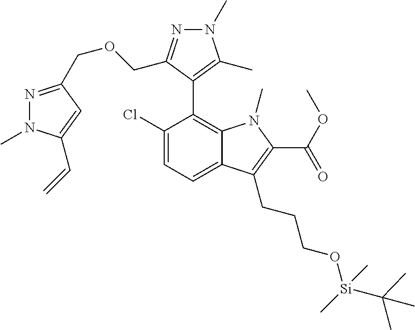 [SMILES: C=Cc1cc(COCc2nn(C)c(C)c2-c2c(Cl)ccc3c(CCCO[Si](C)(C)C(C)(C)C)c(C(=O)OC)n(C)c23)nn1C]

A solution of Intermediate 16 (320 mg, 0.369 mmol), vinylboronic acid pinacol ester (0.1 mL, 1.6 eq.) and Na₂CO₃ (77 mg, 2 eq.) in water (0.75 mL) and 1,4-dioxane (2 mL), in a microwave vial, was degassed under a flow of nitrogen for 10 min before PdCl₂(dtbpf) (CAS [95408-45-0], 70 mg, 0.3 eq.) was added. The solution was degassed for a further 2 min and the vial was sealed stirred at 100° C. under microwave irradiation for 2.5 h. The reaction mixture was cooled to room temperature and was diluted with EtOAc (20 mL) and water (20 mL). The layers were separated and the aqueous layer was extracted with EtOAc (3×20 mL). The combined organic layer was washed with brine (10 mL), dried over MgSO₄, filtered, and concentrated. The residue was purified by flash column chromatography (SiO₂, 8 g column, 0-7% MeOH in DCM). The pure fractions were combined and concentrated to afford Intermediate 17 (0.22 g, yield: 93%) as a brown oil.

### Intermediate 18

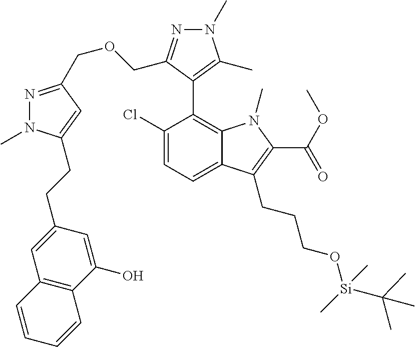 [SMILES: COC(=O)c1c(CCCO[Si](C)(C)C(C)(C)C)c2ccc(Cl)c(-c3c(COCc4cc(CCc5cc(O)c6ccccc6c5)n(C)n4)nn(C)c3C)c2n1C]

9-BBN (0.5 M in TIE, 3.79 mL, 5.5 eq.) was added dropwise to a solution of Intermediate 17 (0.22 g, 0.344 mmol) in THE (0.5 mL) at room temperature. The resulting solution was degassed with nitrogen for 10 min before being sealed in a pressure tube and heated for 30 min at 50° C. under nitrogen atmosphere. The reaction mixture was cooled to room temperature before K₃PO₄ (0.24 g, 3.3 eq.), water (1.2 mL), THE (6 mL), 3-bromo-1-hydroxynaphthalene (0.50 g, 6.5 eq.) and PdCl₂(dtbpf) (CAS [95408-45-0]) (52 mg, 0.2 eq.) were added in that order. The resulting mixture was degassed using a flow of nitrogen for 10 min. The pressure tube was then sealed and heated at 95° C. for 12 h under nitrogen atmosphere. After cooling to room temperature, the solvent was evaporated. The residue was dissolved in EtOAc (20 mL). The layers were separated and the organic layer was washed with water (10 mL). The aqueous layer was extracted with EtOAc (3×20 mL). The combined organic layer was washed with brine, dried over MgSO₄, filtered, and concentrated. The residue was purified using flash column chromatography (SiO₂, 12 g column, 0-7% MeOH in DCM) and the fractions containing product were combined and concentrated to afford Intermediate 18 (0.18 g, yield: 66%) as a pale brown oil.

### Intermediate 19

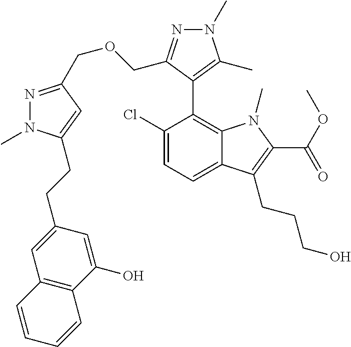 [SMILES: COC(=O)c1c(CCCO)c2ccc(Cl)c(-c3c(COCc4cc(CCc5cc(O)c6ccccc6c5)n(C)n4)nn(C)c3C)c2n1C]

TBAF (1 M in THF, 0.37 mL, 1.6 eq.) was added to a solution of Intermediate 18 (0.18 g, 0.23 mmol) in anhydrous THF (4 mL) at room temperature. The reaction mixture was stirred at room temperature for 2 h. The reaction mixture was concentrated and the residue was dissolved in EtOAc (10 mL) and washed with water (5 mL). The aqueous layer was extracted with EtOAc (3×10 mL) and the combined organic layer was washed with brine, dried over MgSO₄, filtered, and concentrated to afford Intermediate 19 (0.16 g, yield: quantitative) as a sticky brown oil, used without further purification.

### Intermediate 20 and Intermediate 21

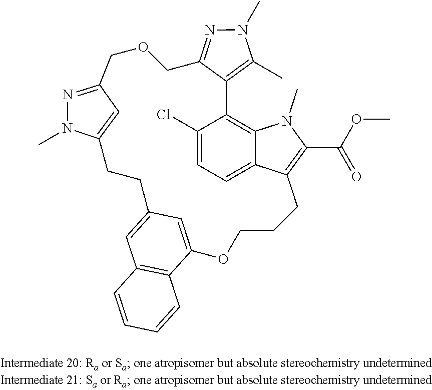

A solution of Intermediate 19 (0.15 g, 0.224 mmol) and DTBAD (206 mg, 4 eq.) in anhydrous toluene (5 mL) and anhydrous THF (3 mL) was degassed under a flow of nitrogen for 10 min. This solution was added via syringe pump (0.1 mL/min) to a degassed solution of PPh₃ (235 mg, 4 eq.) in anhydrous toluene (15 mL) at 70° C. Once the addition was complete, the reaction mixture was diluted with EtOAc (20 mL). The layers were separated and the organic layer was washed with water (10 mL). The aqueous layer was extracted with EtOAc (3×20 mL). The combined organic layer was washed with brine, dried over MgSO₄, filtered, and concentrated. The residue was purified by flash column chromatography (SiO₂, 8 g column, 0-5% MeOH in DCM) and the pure fractions combined and concentrated to afford a mixture of Intermediate 20 and Intermediate 21 (0.06 g, yield: 41%). Several batches of the mixture of Intermediate 20 and Intermediate 21 were combined and further purified by preparative HPLC (Stationary phase: RP XBridge Prep C18 OBD—10 μm, 50×150 mm, Mobile phase: 0.25% NH₄HCO₃ solution in water, CH₃CN). Finally, the purified mixture of Intermediate 20 and Intermediate 21 was separated into its atropisomers by SFC (Stationary phase: Chiralpak Diacel AD 20×250 mm, Mobile phase: CO₂, iPrOH+0.4 iPrNH₂), to afford Intermediate 20 (37 mg, yield: 9%) and Intermediate 21 (41 mg, yield: 10%), as clear oils.

### Intermediate 22

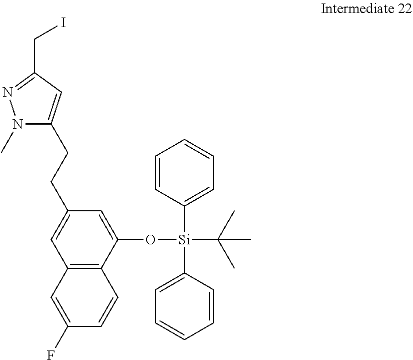 [SMILES: Cn1nc(CI)cc1CCc1cc(O[Si](c2ccccc2)(c2ccccc2)C(C)(C)C)c2ccc(F)cc2c1]

DIPEA (0.64 mL, 2 eq.) followed by methanesulfonic anhydride (0.65 g, 2 eq.) was added to a solution of Intermediate 6 (1.0 g, 1.86 mmol) in THF (45 mL), cooled to 0° C. The reaction mixture was stirred at room temperature for 0.5 h. Sodium iodide (1.39 g, 5 eq.) was then added to the mixture and it was further stirred at room temperature for 1 h. The reaction mixture was diluted with DCM (100 mL) and washed with water (20 mL). The aqueous layer was extracted with DCM/iPrOH 3:1 (2×30 mL), the combined organic layer was dried over MgSO₄, and concentrated under reduced pressure to give a dark yellow oil. This oil was purified by flash column chromatography on silica gel (SiO₂, 24 g column, 0-3% MeOH in DCM) to give Intermediate 22 (1.1 g, yield: 91%)

### Intermediate 23

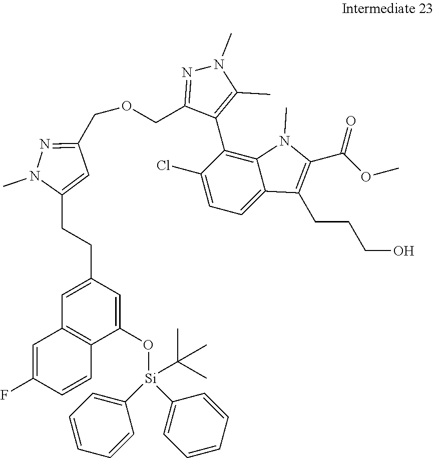 [SMILES: COC(=O)c1c(CCCO)c2ccc(Cl)c(-c3c(COCc4cc(CCc5cc(O[Si](c6ccccc6)(c6ccccc6)C(C)(C)C)c6ccc(F)cc6c5)n(C)n4)nn(C)c3C)c2n1C]

A solution of Intermediate 13 (0.74 g, 1.42 mmol) and Intermediate 22 (1.11 g, 1.2 eq.) in anhydrous THE (5 mL) was added dropwise over 1 h to a cooled (0° C.) suspension of NaH (60% dispersion in mineral oil, 94 mg, 1.65 eq.) in anhydrous THF (6 mL), under nitrogen atmosphere. The reaction mixture was stirred at room temperature for 12 h. The reaction mixture was concentrated in vacuo to approximately 1/5 volume and was redissolved in DCM (20 mL) and water (15 mL). The layers were separated and the aqueous layer was extracted again with iPrOH/DCM (25/75, 3×30 mL), and the combined organic layer was dried over MgSO₄, filtered, and concentrated in vacuo to afford Intermediate 23 (1.7 g, considered quantitative) as a sticky brown solid, used without further purification.

### Intermediate 24

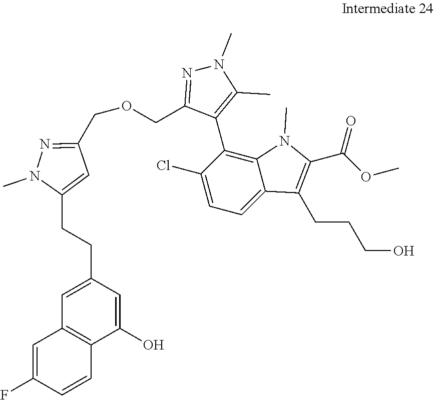 [SMILES: COC(=O)c1c(CCCO)c2ccc(Cl)c(-c3c(COCc4cc(CCc5cc(O)c6ccc(F)cc6c5)n(C)n4)nn(C)c3C)c2n1C]

TBAF (1 M in THF, 3.67 mL, 2 eq.) was added dropwise to a solution of Intermediate 23 5 (1.7 g, 1.84 mmol) in dry THF (5 mL) under nitrogen atmosphere at 0° C. After 4 h, the reaction mixture was cooled to 0° C., diluted with Et₂O (20 mL) and water (20 mL). After further dilution with EtOAc (100 mL), the layers were separated, and the aqueous layer was extracted again with EtOAc (3×50 mL). The combined organic layer was washed with brine (30 mL), dried over MgSO₄, filtered, and concentrated in vacuo. The crude product was purified using flash column chromatography (SiO₂, 80 g column, 0-4% MeOH in DCM) to afford Intermediate 24 (0.35 g, yield: 27% over 2 steps) as an off white solid.

### Intermediate 25 and Intermediate 26

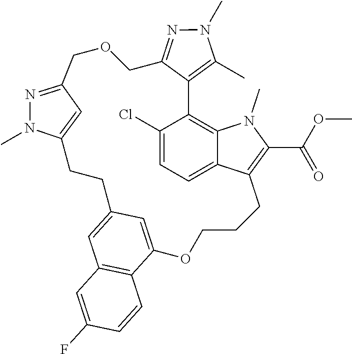

### Intermediate 25: Rₐ or Sₐ; One Atropisomer but Absolute Stereochemistry Undetermined

### Intermediate 26: Sₐ or Rₐ; One Atropisomer but Absolute Stereochemistry Undetermined

A solution of Intermediate 24 (350 mg, 0.51 mmol) in anhydrous toluene (14 mL) and anhydrous THE (3 mL) was degassed under a flow of nitrogen for 10 min, before di-tert-butyl azodicarboxylate (0.47 g, 4 eq.) was added. This solution was degassed for a further 10 min before it was added via syringe pump (0.2 mL/min) to a solution of PPh₃ (0.52 g, 4 eq.) in degassed anhydrous toluene (30 mL) at 70° C. Once the addition was complete, the reaction mixture was concentrated to ⅕th volume before being diluted with EtOAc (100 mL). The organic layer was washed with water (15 mL), followed by brine, dried over MgSO₄, filtered, and concentrated in vacuo. The crude product was purified using flash column chromatography (SiO₂, 12 g column, 0-4% MeOH in DCM) to give a mixture of Intermediate 25 and Intermediate 26. This mixture was separated into its individual atropisomers, by preparative SFC (Stationary phase: Chiralcel Diacel OJ 20×250 mm, Mobile phase: CO₂, MeOH+0.4 iPrNH₂), to afford Intermediate 25 (56 mg, yield: 16%) and Intermediate 26 (55 mg, yield: 16%).

### Intermediate 27

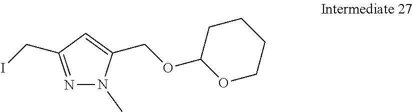 [SMILES: Cn1nc(CI)cc1COC1CCCCO1]

Et₃N (2.396 mL3 eq.) was added to 1-methyl-5-[[(tetrahydro-2H-pyran-2-yl)oxy]methyl]-1H-pyrazole-3-methanol (CAS [2245716-15-6]) (1.3 g, 5.745 mmol) in DCM (40 mL). The resulting reaction mixture was cooled to 0° C. before slow addition of MsCl (1.115 mL, 2.5 eq.). Once the addition was complete, the reaction mixture was allowed to warm to room temperature and was stirred for 3 h. The reaction was concentrated under reduced pressure to give a yellow oil. This oil was partitioned between EtOAc (15 mL) and saturated aqueous NaHCO₃ (10 mL). The layers were separated and the aqueous layer was extracted with EtOAc (15 mL). The combined organic layer was washed with brine, dried over MgSO₄, filtered, and concentrated under reduced pressure to give a yellow oil. This oil was dissolved in ACN (20 mL) and NaI (1.5 g, 1.75 eq.) was added. The reaction mixture was stirred at room temperature overnight. The reaction mixture was filtered through a pad of celite and concentrated under reduced pressure. The crude product was purified by flash column chromatography on silica gel (heptane:EtOAc—1:0 to 3:1) to give Intermediate 27 (1.38 g, yield: 68%) as a yellow oil.

### Intermediate 28

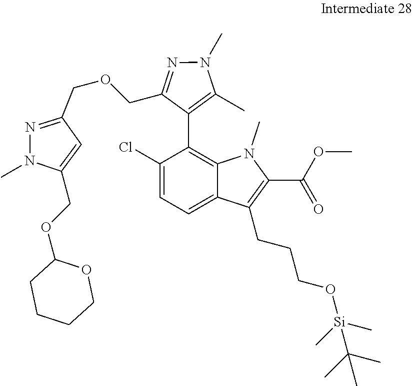 [SMILES: COC(=O)c1c(CCCO[Si](C)(C)C(C)(C)C)c2ccc(Cl)c(-c3c(COCc4cc(COC5CCCCO5)n(C)n4)nn(C)c3C)c2n1C]

NaH (60% dispersion in mineral oil, 115 mg, 1.5 eq.) was added to a solution of Intermediate 13 (1 g, 1.923 mmol) in DMF (18 mL) stirring at 0° C. under nitrogen atmosphere. The reaction mixture was stirred at 0° C. for 10 min, then the ice bath was removed and a solution of Intermediate 27 (1 g, 1.55 eq.) in DMF (7 mL) was added via syringe pump (0.15 mL/min). After the addition, the reaction mixture was stirred at room temperature for a further 16 h. The reaction was quenched by addition of water (50 mL) and diluted with EtOAc (100 mL). The organic layer was separated and washed with brine (3×30 mL). The combined aqueous layer was extracted with EtOAc (50 mL). The combined organic layer was dried over MgSO₄, filtered, and evaporated. The residue was purified by flash chromatography on silica gel (80 g, gradient: from DCM 100% to DCM/MeOH 96/4) to afford Intermediate 28 (1.125 g, yield: 80%) as a yellowish paste.

### Intermediate 29

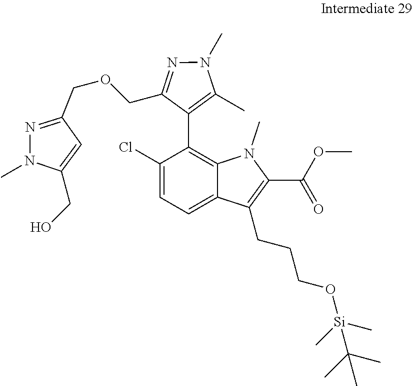 [SMILES: COC(=O)c1c(CCCO[Si](C)(C)C(C)(C)C)c2ccc(Cl)c(-c3c(COCc4cc(CO)n(C)n4)nn(C)c3C)c2n1C]

MgBr₂ (2.53 g, 10 eq.) was added to a solution of Intermediate 28 (1 g, 1.373 mmol) in Et₂O. The reaction mixture was stirred at room temperature for 45 min. Then, more MgBr₂ (0.5 g, 2 eq.) was added and the reaction mixture was stirred for an additional 5 min. The reaction mixture was diluted with EtOAc (50 mL) and water (30 mL). The organic layer was separated and washed with water (30 mL). The organic layer was dried over MgSO₄, filtered, and evaporated. The residue was purified by flash chromatography on silica gel (40 g, gradient: from DCM 100% to DCM/MeOH 95/5) to afford Intermediate 29 (715 mg, yield: 81%) as a colorless paste.

### Intermediate 30

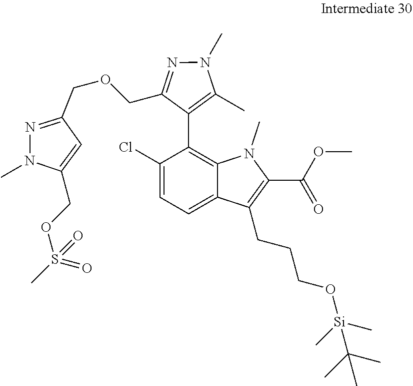 [SMILES: COC(=O)c1c(CCCO[Si](C)(C)C(C)(C)C)c2ccc(Cl)c(-c3c(COCc4cc(COS(C)(=O)=O)n(C)n4)nn(C)c3C)c2n1C]

MsCl (215 μL, 2.5 eq.) was added dropwise to a solution of Intermediate 29 (715 mg, 1.11 mmol) and TEA (463 μL, 3 eq.) in DCM (25 mL) stirring at 0° C. under nitrogen atmosphere. The reaction mixture was then allowed to warm up to room temperature and stirred for 1 h. The reaction mixture was diluted with DCM (25 mL) and treated with saturated aqueous NaHCO₃ (20 mL). The organic layer was separated and the aqueous one was extracted with DCM (25 mL). The combined organic layer was dried over MgSO₄, filtered, and evaporated to give Intermediate 30 (assumed quantitative), used without further purification.

### Intermediate 31 and Intermediate 32

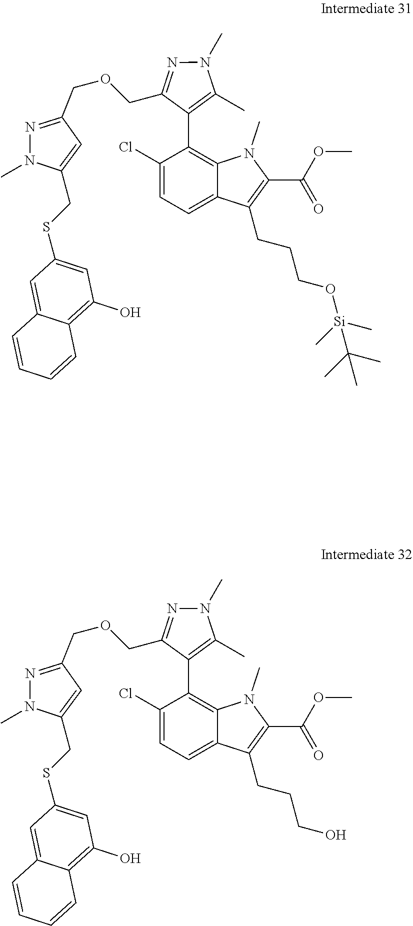

K₂CO₃ (201 mg, 1.5 eq.) was added to a solution of ethanethioic acid, S—[4-(acetyloxy)-2-naphthalenyl] ester (CAS [2143010-96-0]) (328 mg, 1.3 eq.) in degassed MeOH. After 5 min, a solution of crude Intermediate 30 (700 mg, 0.969 mmol) in THF (5 mL) was added dropwise. The reaction mixture was stirred at room temperature for 1 h. More K₂CO₃ (201 mg, 1.5 eq.) was added. The solvents were evaporated and the residue was dissolved in EtOAc (50 mL) and water (30 mL). The organic layer was separated and the aqueous one was extracted with EtOAc (50 mL). The combined organic layer was dried over MgSO₄, filtered, and evaporated. The residue was purified by flash chromatography on silica gel (80 g, gradient: from DCM 100% to DCM/MeOH(NH₃) 95/5) to afford Intermediate 31 (420 mg, yield: 54%) and Intermediate 32 (110 mg, yield: 16%), both as foamy solids.

To convert it to Intermediate 32, Intermediate 31 (420 mg, 0.523 mmol) was dissolved in dry THF (10 mL) and TBAF (1 M in THF, 680 μL, 1.3 eq.) was added while stirring at 0° C. under nitrogen atmosphere. The reaction mixture was stirred at room temperature for 1 h, before more TBAF (1 M in TIE, 680 μL, 1.3 eq.) was added and the reaction mixture was stirred for 3 h. The reaction mixture was concentrated under reduced pressure. The residue was dissolved in EtOAc (30 mL) and washed with water (20 mL) and brine (20 mL). The organic layer was dried over MgSO₄, filtered, and evaporated. The residue was purified by flash column chromatography on silica gel (40 g, gradient: from DCM 100% to DCM/MeOH 95/5) to afford another batch of Intermediate 32 (270 mg, yield: 40% over 2 steps) as a foamy solid.

### Intermediate 33 and Intermediate 34

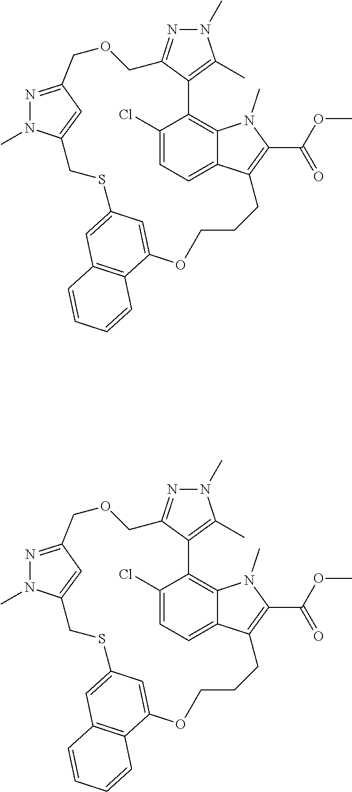

### Intermediate 33: Rₐ or Sₐ; One Atropisomer but Absolute Stereochemistry Undetermined

### Intermediate 34: Sₐ or Rₐ; One Atropisomer but Absolute Stereochemistry Undetermined

A solution of Intermediate 32 (380 mg, 0.552 mmol) and DTBAD (508 mg, 4 eq.) in toluene (12 mL) and THE (2 mL) was added with a syringe pump (0.1 mL/min) to a solution of PPh₃ (579 mg, 4 eq.) in toluene (12 mL) stirring at 70° C. Once the addition was complete, the reaction mixture was allowed to cool down to room temperature and was concentrated under reduced pressure. The residue was purified by flash column chromatography on silica gel (40 g, gradient: from DCM 100% to DCM/MeOH(NH₃) 97.5/2.5) to yield a foamy white solid. This solid was separated into its atropisomers by preparative SFC (Stationary phase: Chiralcel Diacel OJ 20×250 mm, Mobile phase: CO₂, EtOH+0.4 iPrNH₂) to afford Intermediate 33 (122 mg, yield: 33%) and Intermediate 34 (122 mg, yield: 33%).

### Intermediate 35

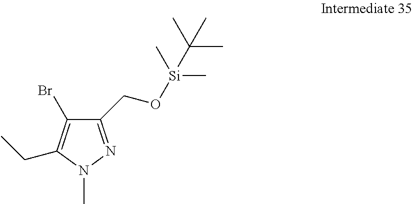 [SMILES: CCc1c(Br)c(CO[Si](C)(C)C(C)(C)C)nn1C]

TBDMSCl (3.436 g, 1.1 eq.) was added in portions to a solution of 4-bromo-5-ethyl-1-methyl-1H-pyrazole-3-methanol (CAS [2138198-53-3]) (4.54 g, 20.72 mmol), DMAP (633 mg, 0.25 eq.), and Et₃N (5.76 mL, 2 eq.) in DCM (100 mL). The reaction mixture was stirred at room temperature overnight. The reaction mixture was diluted with DCM (50 mL) and water (50 mL). The organic layer was separated and the aqueous one was extracted with DCM (50 mL). The combined organic layer was dried over MgSO₄, filtered, and concentrated under vacuum. The residue was purified by flash column chromatography over silica gel (120 g, gradient: from heptane 100% to heptane/EtOAc 7/3) to afford Intermediate 35 (6.22 g, yield: 90%) as a colorless paste.

### Intermediate 36

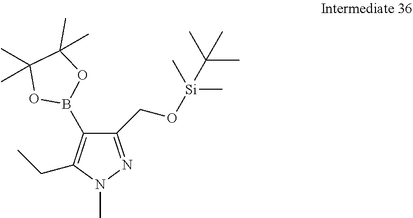 [SMILES: CCc1c(B2OC(C)(C)C(C)(C)O2)c(CO[Si](C)(C)C(C)(C)C)nn1C]

A solution of Intermediate 35 (2 g, 6 mmol) in THF (50 mL) was cooled to −78° C. under nitrogen atmosphere. BuLi (2.5 M in hexane, 3.12 mL, 1.3 eq.) was added dropwise and the mixture was stirred at −78° C. for 45 min. Isopropoxy 4,4,5,5-tetramethyl-1,3,2-dioxaborolane (CAS [61676-62-8]) (1.714 mL, 1.4 eq.) was then added dropwise and the reaction was allowed to warm up to room temperature. The reaction was then quenched with water (50 mL) and diluted with DCM (100 mL). The organic layer was separated and the aqueous one was extracted with DCM (50 mL). The combined organic layer dried over MgsO₄, filtered, and evaporated. The residue was purified by flash column chromatography on silica gel (80 g, gradient: from heptane 100% up to heptane/EtOAc 7/3) to afford Intermediate 36 (1.526 g, yield: 67%) as a white solid.

### Intermediate 37

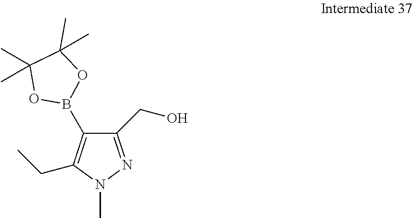 [SMILES: CCc1c(B2OC(C)(C)C(C)(C)O2)c(CO)nn1C]

TBAF (1 M in THF, 4.54 mL, 1.05 eq.) was added dropwise to a solution of Intermediate 36 (1.645 g, 4.324 mmol) in dry THE (35 mL) stirring at 0° C. under nitrogen atmosphere. After 30 min, the reaction was allowed to warm up to room temperature and stirred overnight. The reaction mixture was diluted with EtOAc (50 mL) and water (10 ml). The organic layer was separated and the aqueous one was extracted with EtOAc (2×10 mL). The combined organic layer was dried over MgSO₄, filtered, and evaporated. The crude product was purified by flash column chromatography (40 g, gradient: from DCM 100% to DCM/MeOH 96/4) to afford Intermediate 37 (990 mg, 86%)s as a white solid.

### Intermediate 38

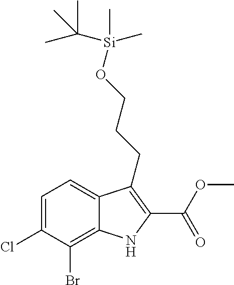 [SMILES: COC(=O)c1[nH]c2c(Br)c(Cl)ccc2c1CCCO[Si](C)(C)C(C)(C)C]

Tert-butyldimethylsilyl chloride (2.06 g, 1.4 eq.) was added portionwise to a mixture of methyl 7-bromo-6-chloro-3-(3-hydroxypropyl)-1H-indole-2-carboxylate (CAS [2245716-18-9]) (3.5 g, 9.78 mmol) and imidazole (1 g, 1.5 eq.) in DCM (80 mL) at 0° C. DMAP (59 mg, 0.05 eq.) was then added and the reaction mixture was stirred at room temperature for 1 h. The reaction mixture was diluted with DCM and washed with water. The organic layer was separated, dried on MgSO₄, filtered, and evaporated to give Intermediate 38 (4.46 g, 87% yield), used without further purification.

### Intermediate 39

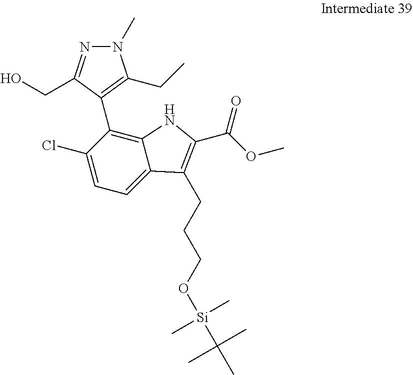 [SMILES: CCc1c(-c2c(Cl)ccc3c(CCCO[Si](C)(C)C(C)(C)C)c(C(=O)OC)[nH]c23)c(CO)nn1C]

A mixture of Intermediate 38 (1.2 g, 2.604 mmol), Intermediate 37 (901 mg, 1.3 eq.), and K₂CO₃ (720 mg, 2 eq.) in dioxane (12 mL) and water (3 mL) was degassed by bubbling nitrogen for a few minutes. Pd(dtbpf)Cl₂ (CAS [95408-45-0]) (85 mg, 0.05 eq.) was added and the reaction mixture was stirred at 80° C. for 2 h. The reaction mixture was diluted with EtOAc (100 mL) and water (50 mL). The aqueous layer was separated and extracted with EtOAc (50 mL). The combined organic layer was dried over MgSO₄, filtered, and evaporated. The residue was purified by flash column chromatography on silica gel (40 g, gradient: from DCM 100% to DCM/MeOH 95/5) to give Intermediate 39 (1.365 g, quantitative) as a yellowish foam.

### Intermediate 40

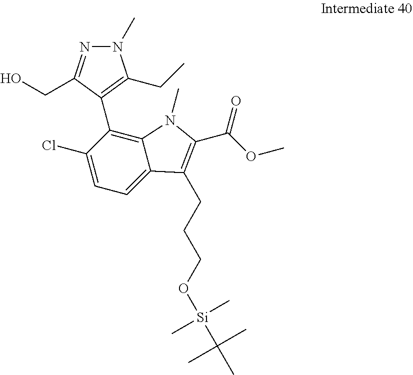 [SMILES: CCc1c(-c2c(Cl)ccc3c(CCCO[Si](C)(C)C(C)(C)C)c(C(=O)OC)n(C)c23)c(CO)nn1C]

Mel (180 μL, 1.1 eq.) was added dropwise to a suspension of Intermediate 39 (1.365 g, 2.624 mmol) and Cs₂CO₃ (1.71 g, 2 eq.) in DMF (8 mL). The reaction mixture was stirred at room temperature for 2 h. The reaction mixture was diluted with EtOAc (60 mL) and water (50 mL). The aqueous layer was separated and the organic one was washed with brine (2×25 mL). The combined aqueous layer was back-extracted with EtOAc (50 mL). The combined organic layer was dried over MgSO₄, filtered, and evaporated. The crude product was purified by flash column chromatography on silica gel (40 g, gradient: from heptane 100% to heptane/EtOAc 3/7) to give Intermediate 40 (1.086 g, yield: 77%) as a yellowish solid.

### Intermediate 41 and Intermediate 42

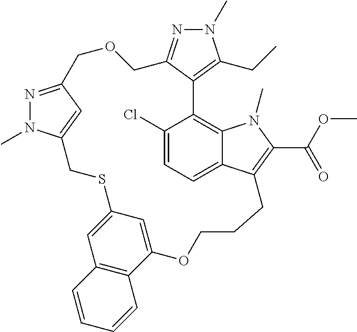

### Intermediate 41: Rₐ or Sₐ; One Atropisomer but Absolute Stereochemistry Undetermined

### Intermediate 42: Sₐ or Rₐ; One Atropisomer but Absolute Stereochemistry Undetermined

Intermediate 41 and Intermediate 42 were prepared via analogous procedures as Intermediate 33 and Intermediate 34, respectively, starting from Intermediate 40 instead of Intermediate 13.

### Intermediate 43

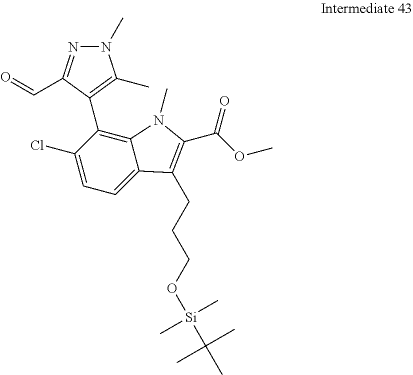 [SMILES: COC(=O)c1c(CCCO[Si](C)(C)C(C)(C)C)c2ccc(Cl)c(-c3c(C=O)nn(C)c3C)c2n1C]

Dess-Martin periodinane (CAS [87413-09-0]) (6.12 g, 1.5 eq.) was added to a solution of Intermediate 13 (5 g, 9.613 mmol) in DCM (100 mL) at room temperature and the reaction mixture was stirred for 2 h. Saturated aqueous NaHCO₃ was added and the resulting solution was extracted with DCM (2×100 mL). The combined organic layer was washed with brine, dried over MgSO₄, and concentrated under reduced pressure. The residue was purified by column chromatography on silica gel (heptane/EtOAc) to give Intermediate 43 (4.58 g, yield: 92%) as a pale yellow oil.

### Intermediate 44

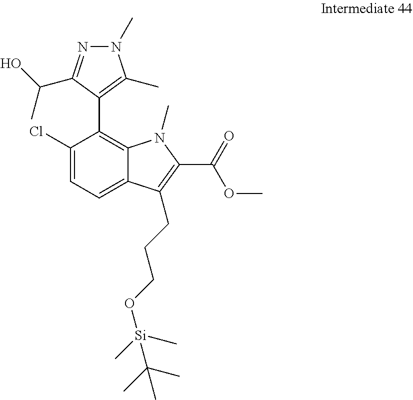 [SMILES: COC(=O)c1c(CCCO[Si](C)(C)C(C)(C)C)c2ccc(Cl)c(-c3c(C(C)O)nn(C)c3C)c2n1C]

Methylmagnesium bromide (3.4 M in THF, 2.9 mL, 2.6 eq.) was added dropwise to an ice-cooled, stirred solution of Intermediate 43 (1.95 g, 3.76 mmol) in THF (30 mL). After 2 h at 0° C., the reaction was quenched with saturated aqueous NH₄Cl, diluted with water and EtOAc. The layers were separated, the organic layer was treated with brine, dried on MgSO₄, filtered, and evaporated to afford Intermediate 44 (2 g, yield: 99%) as an oil.

### Intermediate 45

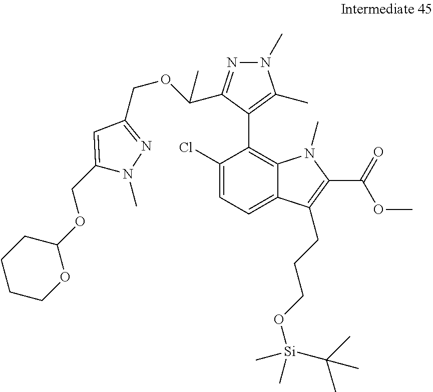 [SMILES: COC(=O)c1c(CCCO[Si](C)(C)C(C)(C)C)c2ccc(Cl)c(-c3c(C(C)OCc4cc(COC5CCCCO5)n(C)n4)nn(C)c3C)c2n1C]

A solution of Intermediate 44 (2 g, 3.74 mmol) in THE (10 mL) was added dropwise to a stirred suspension of NaH (60% dispersion in mineral oil, 0.24 g, 1.6 eq.) in dry THF (30 mL) at room temperature. After the addition was complete, stirring was continued for 30 min before adding dropwise a solution of Intermediate 27 (2.14 g, 1.7 eq.) in THF (10 mL). After 16 h at room temperature, the solution was partitioned between EtOAc/saturated aqueous NH₄Cl. The layers were separated, and the aqueous layer was extracted with EtOAc. The combined organic layer was treated with brine, dried on MgSO₄, filtered, and concentrated in vacuo to afford a tan oil. This oil was purified by flash column chromatography (SiO₂, 0-10% MeOH in DCM) to afford Intermediate 45 (1.95 g, yield: 70%) as a yellowish oil.

### Intermediate 46

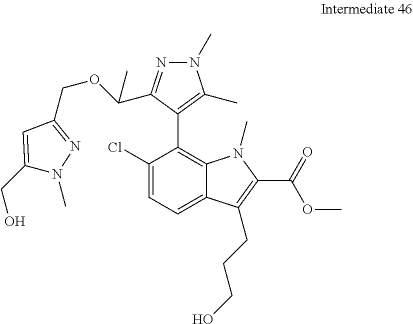 [SMILES: COC(=O)c1c(CCCO)c2ccc(Cl)c(-c3c(C(C)OCc4cc(CO)n(C)n4)nn(C)c3C)c2n1C]

MgBr₂ (4.84 g, 10 eq.). was added to a stirred solution of Intermediate 45 (1.95 g, 2.63 mmol) in Et₂O (100 mL). After 1 h of stirring, more MgBr₂ (4.84 g, 10 eq.) was added and stirring was continued for 16 h at room temperature. The brown suspension was diluted with EtOAc and water and the layers were separated. The aqueous layer was extracted with EtOAc and the combined organic layer was treated with brine, dried on MgSO₄, filtered, and concentrated in vacuo to afford an oil. This oil was purified by flash column chromatography (SiO₂, 0-10% MeOH in DCM) to afford Intermediate 46 (0.72 g, yield: 50%) as an oil.

### Intermediate 47

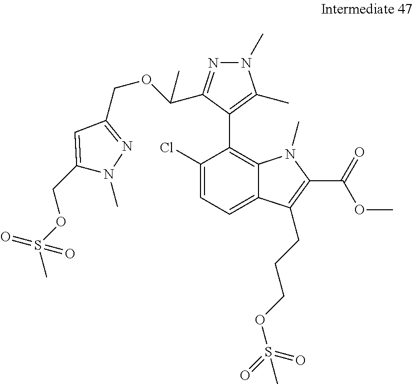 [SMILES: COC(=O)c1c(CCCOS(C)(=O)=O)c2ccc(Cl)c(-c3c(C(C)OCc4cc(COS(C)(=O)=O)n(C)n4)nn(C)c3C)c2n1C]

MsCl (0.41 mL, 4 eq.) was added dropwise to an ice-cooled stirred solution of Intermediate 46 (0.72 g, 1.32 mmol) and TEA (1.1 mL, 6 eq.) in THE (25 mL). The ice bath was removed and stirring was continued for 1 h at room temperature. The suspension was diluted with EtOAc, treated with water, and the layers were separated. The aqueous layer was extracted with EtOAc (2×), and the combined organic layer was treated with buffer pH=4 (citric acid/Na₂HPO₄), brine, dried on MgSO₄, filtered, and concentrated in vacuo to afford Intermediate 47 (1.1 g, quantitative) as an oil, used as such without further purification.

### Intermediate 48

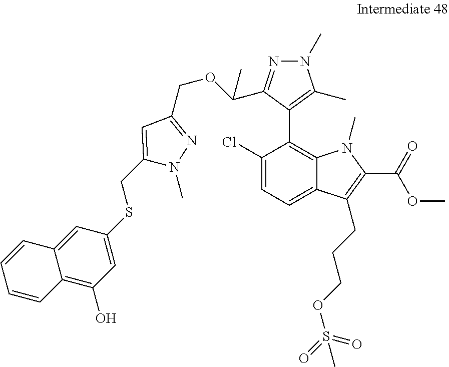 [SMILES: COC(=O)c1c(CCCOS(C)(=O)=O)c2ccc(Cl)c(-c3c(C(C)OCc4cc(CSc5cc(O)c6ccccc6c5)n(C)n4)nn(C)c3C)c2n1C]

K₂CO₃ (0.65 g, 3 eq.) was added to a stirred and thoroughly degassed (nitrogen bubbling for 15 min) solution of Intermediate 47 (1.1 g, 1.57 mmol), 3-(acetylthio)naphthalen-1-yl acetate (CAS [2143010-96-0]) (0.48 g, 1.16 eq.), and PPh₃ (60 mg, 0.14 eq.) in MeOH (25 mL). After 30 min of stirring at room temperature, the suspension was partly concentrated, diluted with DCM and water and the layers were separated. The aqueous layer was extracted with DCM. The combined organic layer was dried over MgSO₄, filtered, and evaporated. The residue was purified by flash column chromatography (SiO₂, 40 g Redisep flash column; 0-5% MeOH in DCM) to afford Intermediate 48 (0.61 g, yield: 49%) as a yellowish oil.

### Intermediate 49, Intermediate 50, Intermediate 51, and Intermediate 52

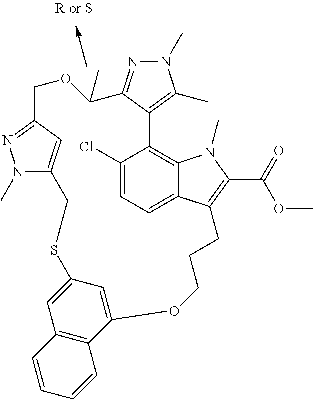

### Intermediate 49: Rₐ or Sₐ; One Atropisomer but Absolute Stereochemistry Undetermined

### Intermediate 50: Sₐ or Rₐ; One Atropisomer but Absolute Stereochemistry Undetermined

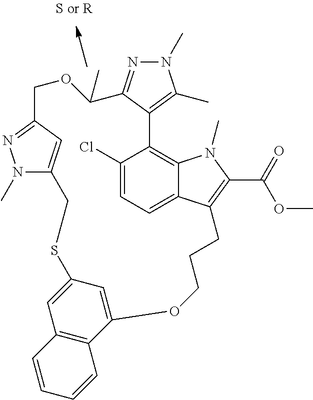

### Intermediate 51: Rₐ or Sₐ; One Atropisomer but Absolute Stereochemistry Undetermined

### Intermediate 52: Sₐ or Rₐ; One Atropisomer but Absolute Stereochemistry Undetermined

A solution of Intermediate 48 (610 mg, 0.78 mmol) in ACN (15 mL) was added dropwise using a syringe pump to a hot (82° C.), stirred, suspension of K₂CO₃ (250 mg, 2.3 eq.) in ACN (50 mL), at a rate of 0.05 mL/min. When the addition was complete, the suspension was concentrated in vacuo, diluted with water and DCM, and the layers were separated.

The organic layer was treated with water, dried on MgSO₄, filtered, and evaporated. The residue was purified by flash column chromatography (40 g Redisep flash column, 0-4% MeOH in DCM). The product (mixture of diastereoisomers) was further purified by preparative HPLC (Stationary phase: RP XBridge Prep C18 OBD-10 μm, 50×150 mm, Mobile phase: 0.25% NH₄HCO₃ solution in water, ACN) to afford two racemic mixtures. The first racemic mixture was further separated into its enantiomers by preparative SFC (Stationary phase: Chiralpak Daicel ID 20×250 mm, Mobile phase: CO₂, EtOH+0.4 iPrNH₂) to give Intermediate 49 (54 mg, yield: 10%) and Intermediate 50 (56 mg, yield: 10%) as sticky solids. The second racemic mixture was further separated into its enantiomers by preparative SFC (Stationary phase: Chiralpak Daicel ID 20×250 mm, Mobile phase: CO₂, EtOH+0.4 iPrNH₂) to give Intermediate 51 (25 mg, yield: 5%) and Intermediate 52 (34 mg, yield: 6%) as sticky solids.

### Intermediate 53

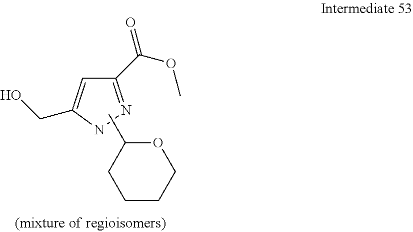

A solution of 1H-pyrazole-3,5-dicarboxylic acid, 1-(tetrahydro-2H-pyran-2-yl)-, 3,5-dimethyl ester (CAS [406486-55-3]) (6.09 g, 22.7 mmol) in dry MeOH (22 mL) and dry Me-THF (32 mL) was cooled to 0° C. under nitrogen atmosphere. NaBH₄ (536 mg, 0.6 eq.) was added at 0° C. in four portions over 10 min. The reaction mixture was stirred at 0° C. for 30 min then at room temperature for 3.5 h. The reaction mixture was cooled to 0° C. and additional NaBH₄ (400 mg, 0.46 eq.) was added. The reaction mixture was stirred at room temperature for 2.5 h. The reaction was quenched by slow addition of acetone, water and EtOAc. The layers were separated and the aqueous layer was extracted with EtOAc (×3). The combined organic layer was dried over MgSO₄, filtered, and evaporated. The residue was purified by flash column chromatography (SiO₂, 120 g RediSep, DCM/MeOH, 100/0 to 95/5) to afford Intermediate 53 (6.2 g, yield: 88%) as a pale yellow oil.

### Intermediate 54

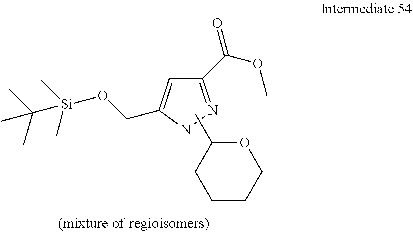

A solution of Intermediate 53 (3.43 g, 11.14 mmol) and imidazole (1.06 g, 1.4 eq.) in dry DCM (20 mL) was cooled to 0° C. under nitrogen atmosphere. TBDMSCl (2.01 g, 1.2 eq.) was added in two portions over 1 min, leading to a white suspension. The reaction mixture was stirred at 0° C. for 30 min, then at room temperature for 17 h. The reaction was quenched by addition of saturated aqueous NH₄Cl. The layers were separated, the organic layer was washed with saturated aqueous NH₄Cl, the combined aqueous layer was extracted with DCM (×3), and the combined organic layer was dried over MgSO₄, filtered, and evaporated. The resulting colorless oil was purified by flash column chromatography (SiO₂, 80 g RediSep, EtOAc in n-heptane 0/100 to 40/60) to yield Intermediate 54 (3.86 g, yield: 81%) as a colorless oil.

### Intermediate 55

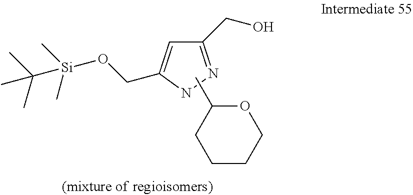

A solution of Intermediate 54 (3.86 g, 10.89 mmol) in dry THE (44 mL) was cooled to 0° C. under nitrogen atmosphere. LiAlH₄ (2 M solution in THF, 6.21 mL, 1.14 eq.) was added dropwise under nitrogen atmosphere. The reaction mixture was stirred at 0° C. for 1 h. The reaction was quenched by slow addition of EtOAc followed by addition of a saturated aqueous solution of Rochelle salt. This mixture was stirred at room temperature for 5 min. The layers were separated and the aqueous layer was extracted with EtOAc (×3). The combined organic layer was washed with brine (×2), dried over MgSO₄, filtered, and evaporated to yield Intermediate 55 (3.54 g, yield 99%) as a colourless oil.

### Intermediate 56

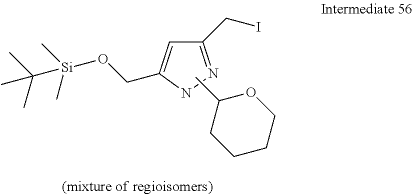

A solution of Intermediate 55 (3.37 g, 10.32 mmol) in dry DCM (72 mL) was cooled to 0° C. under nitrogen atmosphere. TEA (4.3 mL, 3 eq.) was added, followed by dropwise addition of MsCl (2 mL, 2.5 eq.). Once the addition was complete, the reaction mixture was allowed to warm to room temperature and was stirred for 3.5 h. The reaction mixture was concentrated under reduced pressure. The residue was partitioned between EtOAc and saturated aqueous. NaHCO₃. The layers were separated and the aqueous layer was extracted with EtOAc (×3). The combined organic layer was washed with brine (×2), dried over MgSO₄, filtered, and evaporated to give a yellow oil. This oil was dissolved in dry ACN (65 mL) under nitrogen atmosphere. NaI (2.71 g, 1.75 eq.) was added and the reaction mixture was stirred at room temperature overnight. The reaction mixture was filtered through a pad of Celite®. The filtrate was washed with a saturated aqueous Na₂S₂O₃ solution, dried over MgSO₄, filtered, and evaporated. The residue was purified by flash column chromatography (SiO₂, 120 g RediSep, heptane/EtOAc, 100/0 to 0/100) to give Intermediate 56 (3.68 g, yield: 80%) as a yellow oil.

### Intermediate 57

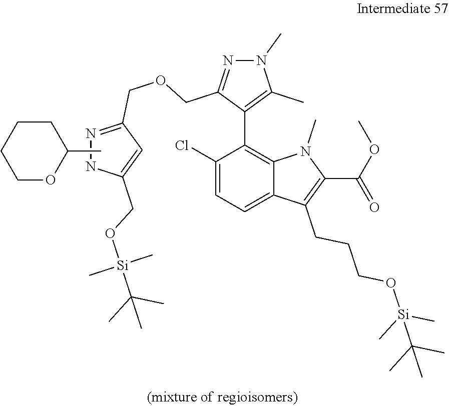

A solution of Intermediate 56 (3.55 g, 1.05 eq.) and Intermediate 13 (4.08 g, 7.747 mmol) in dry THE (60 mL) was added dropwise over 20 min to a suspension of NaH (60% dispersion in mineral oil, 465 mg, 1.5 eq.) in dry THE (20 mL) at 0° C. under nitrogen atmosphere. The reaction mixture was stirred at 0° C. for 1 h then at room temperature for 3 h. The reaction was quenched by adding a drop of MeOH followed by a drop of saturated aqueous NH₄Cl. The mixture was diluted with EtOAc and water and brine were added. The layers were separated. The organic layer was washed with brine and the combined aqueous layer was extracted with EtOAc (×3). The combined organic layer was dried over MgSO₄, filtered, and evaporated. The crude mixture was purified by flash column chromatography (SiO₂, 120 g RediSep, DCM/MeOH, 100/0 to 90/10) to afford Intermediate 57 (5.6 g, yield: 79%) as a thick pale yellow oil.

### Intermediate 58

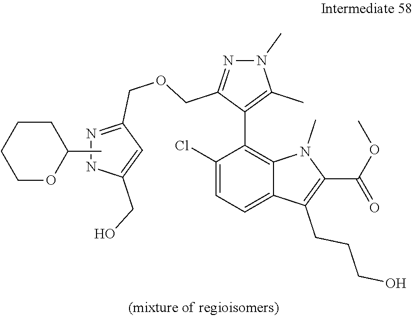

TBAF (1 M solution in THF, 16.89 mL, 2.5 eq.) was added to a solution of Intermediate 57 (5.6 g, 6.758 mmol) in dry THE (127 mL) at 0° C. under nitrogen atmosphere. The ice bath was removed and the reaction mixture was stirred at room temperature for 40 min. The reaction was quenched by addition of saturated aqueous NH₄Cl and the layers were separated. The organic layer was washed with brine (×2), and the combined aqueous layer was extracted with EtOAc (×3) and DCM. The combined organic layer was dried over MgSO₄, filtered, and evaporated to give a brown oil. This oil was purified by flash column chromatography (SiO₂, 120 g RediSep, DCM/MeOH, 100/0 to 90/1) to afford a pale yellow oil that solidified upon addition of diisopropylether to yield, after filtration, Intermediate 58 (3.58 g, yield: 59%) as a pale yellow solid.

### Intermediate 59

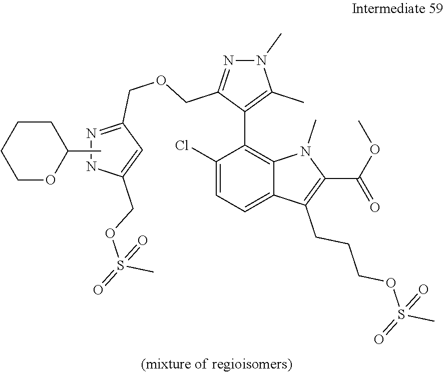

Et₃N (1.04 mL, 3 eq.) was added to a solution of Intermediate 58 (1.5 g, 2.5 mmol) in dry THF (31 mL). The reaction mixture was cooled to 0° C. before slow addition of MsCl (0.48 mL, 2.5 eq.). Once the addition was complete, the reaction mixture was allowed to warm to room temperature and was stirred for 1.5 h. The reaction mixture was diluted with EtOAc and water was added. The layers were separated and the aqueous layer was extracted with EtOAc (×3). The combined organic layer was washed with saturated aqueous NH₄Cl, brine, dried over MgSO₄, filtered, and concentrated under reduced pressure (bath at 30° C.) to afford Intermediate 59 as a pale yellow foam, used as such without further purification.

### Intermediate 60

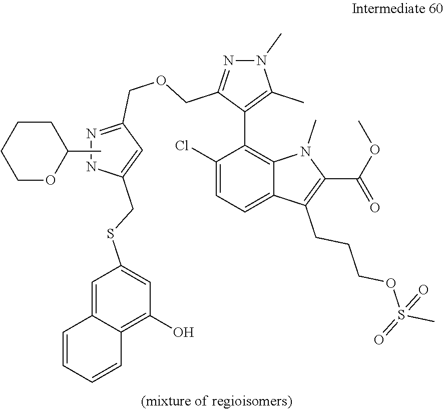

Crude Intermediate 59 (1.89 g, 2.5 mmol) was dissolved in MeOH (77 mL) and THF (8 mL) under nitrogen atmosphere. The reaction mixture was re-filled with nitrogen twice and degassed by bubbling nitrogen for 10 min. 3-(acetylthio)naphthalen-1-yl acetate (CAS [2143010-96-0]) (748 mg, 1.15 eq.) and PPh₃ (65 mg, 0.1 eq.) were added to the reaction mixture which was re-filled with nitrogen twice and degassed by bubbling nitrogen for 10 min. Once all the reagents were in solution, the reaction mixture was cooled to 0° C. before addition of K₂CO₃ (864 mg, 2.5 eq.). The reaction mixture was again re-filled with nitrogen twice and degassed by bubbling nitrogen for 10 min. The reaction mixture was stirred at 0° C. for 1.5 h. The reaction mixture was diluted with DCM and water. The layers were separated and the aqueous layer was extracted with DCM (×3). The combined organic layer was washed with saturated aqueous NH₄Cl and brine, dried over MgSO₄, filtered, and concentrated under reduced pressure to afford a thick red oil. This oil was purified by flash column chromatography (SiO₂, 40 g RediSep, Heptane/EtOAc, 100/0 to 0/100) to afford a thick colorless oil that solidified upon addition of diisopropyl ether to give Intermediate 60 (1.56 g, yield: 48%).

### Intermediate 61

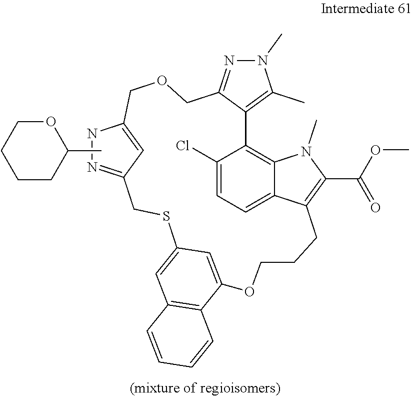

Intermediate 60 (3.02 g, 3.611 mmol) was dissolved in dry ACN (20 mL) under nitrogen atmosphere. The resulting solution was added via syringe pump (0.05 mL/min) to a solution of K₂CO₃ (998 mg, 2 eq.) in dry ACN (270 mL) at 82° C. under nitrogen atmosphere. Once the addition was complete, the reaction mixture was stirred for 30 min at 82° C. After cooling, the reaction mixture was filtered and the filtrate was evaporated. The crude product was purified by flash column chromatography (SiO₂, 80 g RediSep, Heptane/EtOAc, 100/0 to 0/100) to give a brownish oil that solidified upon addition of diisopropylether. Filtration gave Intermediate 61 (2.25 g, yield 59%) as a brown solid.

### Intermediate 62 and Intermediate 63

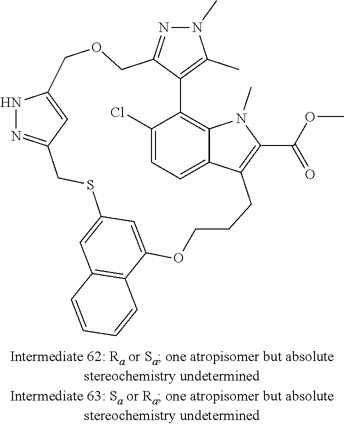 [SMILES: COC(=O)c1c2c3ccc(Cl)c(c3n1C)-c1c(nn(C)c1C)COCc1cc(n[nH]1)CSc1cc(c3ccccc3c1)OCCC2]

HCl (1.25 M in MeOH, 11.89 mL, 50 eq.) was added dropwise to a solution of Intermediate 61 (220 mg, 0.297 mmol) in dry THF (12 mL) at 0° C. The reaction mixture was stirred at room temperature for 1.5 h. The reaction mixture was concentrated under reduced pressure. The residue was purified by preparative SFC (Stationary phase: Chiralcel Diacel OJ 20×250 mm, Mobile phase: CO₂, EtOH+0.4 iPrNH₂) to give Intermediate 62 (46 mg, yield: 23%) and Intermediate 63 (46 mg, yield: 23%), both as pale yellow solids.

### Intermediate 64 and Intermediate 65

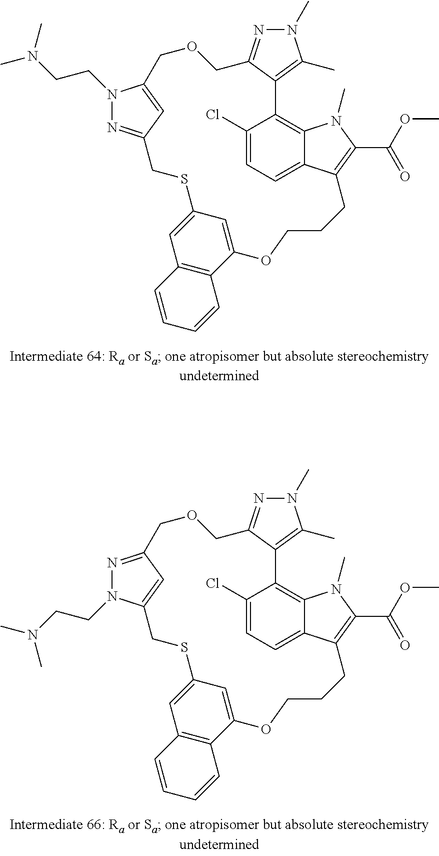

2-Dimethylaminoethyl chloride hydrochloride (CAS [4584-46-7]) (54 mg, 3.5 eq.) was added to a suspension of Intermediate 62 (70 mg, 0.107 mmol) and Cs₂CO₃ (208 mg, 6 eq.) in dry DMF (1 mL) at 25° C. under nitrogen atmosphere. The reaction mixture was stirred at 25° C. for 16 h. The reaction mixture was diluted with EtOAc and water. The organic layer was washed with brine (×3) and the combined aqueous extracts were extracted with EtOAc (×3). The combined organic layer was dried over MgSO₄, filtered, and evaporated. The residue was purified by preparative SFC (Stationary phase: Chiralpak Diacel AD 20×250 mm, Mobile phase: CO₂, EtOH+0.4 iPrNH₂) to give Intermediate 64 (30 mg, yield: 39%) and Intermediate 65 (16 mg, yield: 20%) both as pale yellow foams.

### Intermediate 66

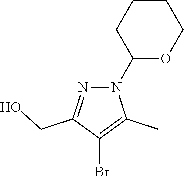

Ethyl 4-bromo-5-methyl-1-(tetrahydro-2H-pyran-2-yl)-1H-pyrazole-3-carboxylate (CAS [2246368-58-9]) (15.35 g, 48.39 mmol) was dissolved in dry 2-Me-THF (200 mL) and cooled to 0° C. LiBH₄ (4 M in THF, 48.39 mL, 4 eq.) was added slowly and the reaction mixture was allowed to warm to room temperature and was stirred at this temperature overnight. The reaction was quenched with water. The water layer was extracted with EtOAc (3×). The combined organic layer was washed with brine, dried with Na₂SO₄, filtered, and solvents were evaporated to afford Intermediate 66 (12.83 g, 96% yield) as a white powder.

### Intermediate 67

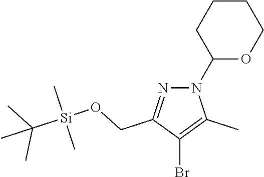 [SMILES: Cc1c(Br)c(CO[Si](C)(C)C(C)(C)C)nn1C1CCCCO1]

To a solution of Intermediate 66 (200 mg, 0.73 mmol) in dry THF (5 mL) under nitrogen atmosphere was added DMAP (35 mg, 0.4 eq.) and Et₃N (0.2 mL, 2 eq.) at room temperature. Then, TBDMSCl (115 mg, 1.05 eq.) was added. To allow full conversion, more TBDMSCl (109 mg, 1 eq.) and Et₃N (0.1 mL, 1 eq.) were added to the reaction mixture and it was stirred for another hour. NaHCO₃ and DCM were added to the reaction mixture. The layers were separated and the aqueous layer was extracted twice with DCM. The combined organic layer was washed with brine, dried with Na₂SO₄, filtered, and evaporated. The residue was purified by flash chromatography [Biotage Isolera 1//Biotage SnapUltra Silica 25 g//EtOAc/Heptane. 0/100 to 40/60] to afford Intermediate 67 (238 mg, 84% yield) as a colorless oil.

### Intermediate 68

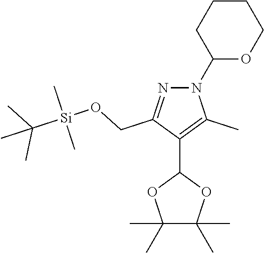 [SMILES: Cc1c(C2OC(C)(C)C(C)(C)O2)c(CO[Si](C)(C)C(C)(C)C)nn1C1CCCCO1]

A solution of Intermediate 67 (5 g, 12.84 mmol) in THF (50 mL) was cooled to −78° C. under nitrogen atmosphere. BuLi (2.5 M in hexane, 7.19 mL, 1.4 eq.) was added dropwise and the mixture was stirred at −78° C. for 20 min. 2-isopropoxy-4,4,5,5-tetramethyl-1,3,2-dioxaborolane (CAS [61676-62-8]) (3.93 mL, 1.5 eq.) was then added and the reaction was allowed to warm to room temperature. After 15 min at room temperature, the reaction was quenched with water and diluted with DCM. The layers were separated. The aqueous layer was extracted with DCM (3×). The combined organic layer was washed with brine, dried with Na₂SO₄, filtered, and evaporated to afford Intermediate 68 (6.01 g, 62% yield) as a colorless oil used without further purification.

### Intermediate 69

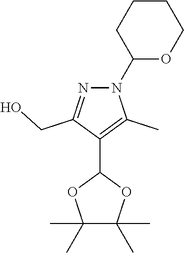 [SMILES: Cc1c(C2OC(C)(C)C(C)(C)O2)c(CO)nn1C1CCCCO1]

To a solution of Intermediate 68 (6.01 g, 7.99 mmol) in dry Me-THF (50 mL) was slowly added TBAF (1 M in THF, 9.58 mL, 1.2 eq.) under nitrogen atmosphere. The reaction mixture was stirred for 15 h. The reaction mixture was diluted with EtOAc, washed with a saturated aqueous NaHCO₃ solution, then with brine, and the combined organic layer was dried with Na₂SO₄, and evaporated. The residue was purified by flash chromatography [Biotage Isolera 1//Biotage SnapUltra Silica 100 g//heptane-EtOAc 100/0 to 80/20] to afford Intermediate 69 (2.43 g, 94% yield) as a white powder.

### Intermediate 71

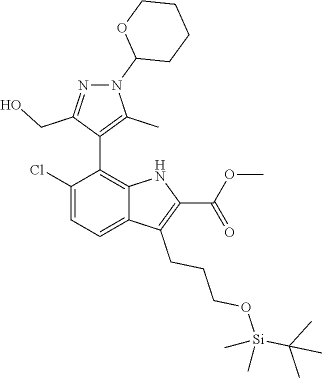 [SMILES: COC(=O)c1[nH]c2c(-c3c(CO)nn(C4CCCCO4)c3C)c(Cl)ccc2c1CCCO[Si](C)(C)C(C)(C)C]

This reaction was performed in 4 batches.

K₃PO₄ (193 g, 3 eq.) and Pd(dtbpf)Cl₂ (19.8 g, 0.1 eq.) were added to a solution of Intermediate 38 (140 g, 304 mmol) and Intermediate 69 (196 g, 2 eq.) in THF (1.6 L) and water (400 mL). The reaction mixture was stirred at room temperature overnight. The reaction was quenched by addition of water (3 L). The resulting mixture was extracted with EtOAc (3×3 L). The combined organic layer was dried over Na₂SO₄, filtered, and concentrated. The residue was purified by silica gel chromatography (EtOAc/petroleum ether 0/100 to 100/0) to afford Intermediate 71 (150 g, yield: 86%) as a yellow oil yield)

### Intermediate 72

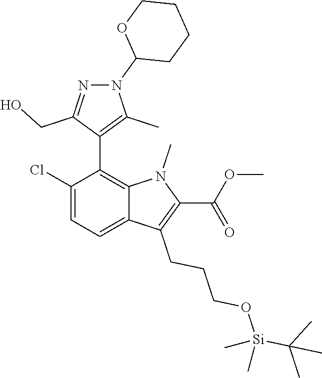 [SMILES: COC(=O)c1c(CCCO[Si](C)(C)C(C)(C)C)c2ccc(Cl)c(-c3c(CO)nn(C4CCCCO4)c3C)c2n1C]

Cs₂CO₃ (19.51 g, 1.5 eq.). was added to a solution of Intermediate 71 (23 g, 39.9 mmol) in ACN (810 mL) and DMF (8 mL). The reaction mixture was stirred at room temperature for 30 min. Methyl iodide (6.8 g, 1.2 eq.) was added to the reaction mixture at 0° C. and the resulting mixture was stirred at room temperature for 3 h. The reaction was quenched by addition of water (100 mL). The resulting mixture was extracted with EtOAc (3×100 mL). The combined organic layer was washed successively with aqueous NaHCO₃, aqueous NH₄Cl, and brine, dried over Na₂SO₄, filtered, and evaporated. The residue was purified by silica gel chromatography (MeOH/CH₂Cl₂ 0/100 to 50/50) to afford Intermediate 72 (20 g, yield: 85%) as a yellow solid.

### Intermediate 73

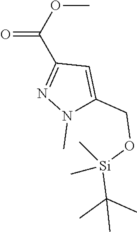 [SMILES: COC(=O)c1cc(CO[Si](C)(C)C(C)(C)C)n(C)n1]

TBDMSCl (76.97 g, 1.1 eq.) and imidazole (34.77 g, 1.1 eq.) were added to a solution of methyl 5-(hydroxymethyl)-1-methyl-1H-pyrazole-3-carboxylate (79 g, 464.25 mmol) in DCM (800 mL). The resulting solution was stirred at room temperature for 16 h. The solvent was evaporated and the residue was purified with silica gel column chromatography (EtOAc/petroleum ether 3/1) to give Intermediate 73 (126 g, yield: 78%) as a light yellow oil.

### Intermediate 74

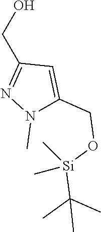 [SMILES: Cn1nc(CO)cc1CO[Si](C)(C)C(C)(C)C]

DIBAL (1 M in hexane, 1330 mL, 3 eq.) was added dropwise to a solution of Intermediate 73 (126 g, 443 mmol) in THF (1000 mL) at 0° C. The resulting solution was stirred at 0° C. for 2 h and then allowed to warm to room temperature. The reaction mixture was carefully poured in a Rochelle salt solution (1500 mL) and EtOAc (1500 mL) was added. The mixture was stirred for 1.5 h. The layers were separated and the aqueous layer was extracted with EtOAc (2×1500 mL). The combined organic layer was dried over MgSO₄, filtered, and evaporated to give Intermediate 74 (108 g, yield: 87%) as a white solid.

### Intermediate 75

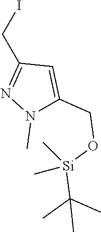 [SMILES: Cn1nc(CI)cc1CO[Si](C)(C)C(C)(C)C]

Methanesulfonic anhydride (20.3 g, 1.3 eq.) was added to a solution of Intermediate 74 (23 g, 89.7 mmol), DIPEA (17.4 g, 1.5 eq.) in THE (200 mL) at 0° C. The reaction mixture was stirred at 0° C. for 5 min, and then at room temperature for 30 min. Sodium iodide (60.5 g, 4.5 eq.) was added and the resulting solution was stirred at 50° C. for 2 h before cooling to room temperature. The reaction mixture was filtered. The filtrate was evaporated and the residue was purified by silica gel column chromatography (EtOAc:petroleum ether 5:1) to give Intermediate 75 (23 g, yield: 66%) as a light yellow oil.

### Intermediate 76

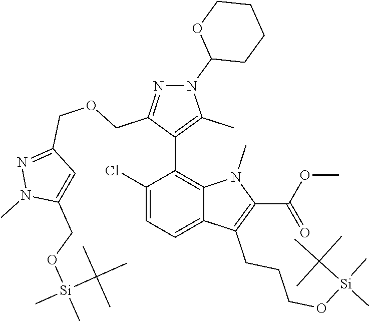

A solution of Intermediate 72 (30 g, 50.8 mmol) and Intermediate 75 (18.6 g, 1 eq.) in THF (300 mL) was added dropwise to a solution of sodium hydride (60% in mineral oil, 1.83 g, 1.5 eq.) in THF (200 mL) at 0° C. The resulting solution was stirred at room temperature for 2 h and the reaction was quenched with saturated aqueous NH₄Cl (150 mL). The layers were separated and the aqueous layer was extracted with EtOAc (300 mL×2). The combined organic layer was dried on MgSO₄, filtered, and evaporated. The residue was purified by silica gel column chromatography (EtOAc:petroleum ether 1:3) to give Intermediate 76 (40 g, yield: 66%) as a light yellow oil.

### Intermediate 77

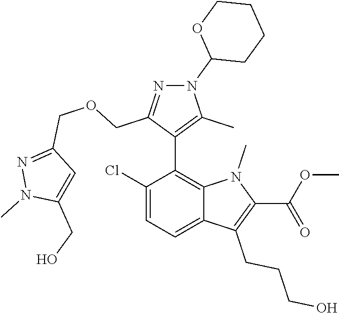

A solution of Intermediate 76 (43 g, 51.9 mmol) and Et₃N·(HF)₃ (CAS [73602-61-6]) (20.9 g, 2.5 eq.) in THF (500 mL) was stirred at room temperature for 16 h. The solvent was evaporated and the residue was partitioned between EtOAc and water. The layers were separated and the organic layer was washed with brine, dried over Na₂SO₄, filtered, and evaporate to afford Intermediate 77 (32 g, quantitative).

### Intermediate 78

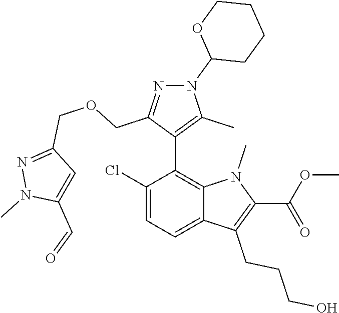

MnO₂ (72.4 g, 20 eq.) was added to a solution of Intermediate 77 (25 g, 41.6 mmol) in CH₂Cl₂ (250 mL). The reaction mixture was stirred at reflux for 16 h. The reaction mixture was filtered and the filtrate was evaporated and dried to give Intermediate 78 (14 g, yield: 54%) as a light yellow oil.

### Intermediate 79

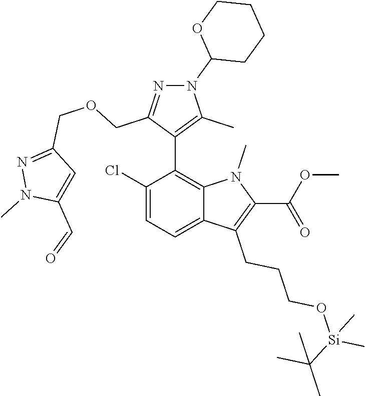

TBDMSCl (4.2 g, 1.2 eq.) and imidazole (1.9 g, 1.2 eq.) were added to a solution of Intermediate 78 (14 g, 23.41 mmol) in CH₂Cl₂ (150 mL). The resulting solution was stirred at room temperature for 3 h. Water (50 mL) was added and the organic layer was separated. The aqueous layer was extracted with DCM (100 mL×2). The combined organic layer was dried on MgSO₄, filtered, and evaporated. The residue was purified by silica gel column chromatography (EtOAc:petroleum ether 1:3) to give Intermediate 79 (11.5 g, yield: 65%) as a light yellow oil.

### Intermediate 80

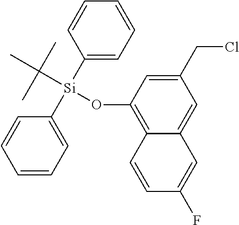 [SMILES: CC(C)(C)[Si](Oc1cc(CCl)cc2cc(F)ccc12)(c1ccccc1)c1ccccc1]

DIPEA (23.5 mL, 4 eq.) and mesyl anhydride (11.75 g, 2 eq.) were added to a solution of Intermediate 2 (14.8 g, 33.7 mmol) in DCM (200 mL) at 0° C. The reaction mixture was stirred at room temperature for 2 h. The reaction mixture was cooled again to 0° C. and LiCl (8.58 g, 6 eq.) was added. The reaction mixture was then stirred at room temperature for 48 h. The reaction was quenched by adding water (200 mL), and the mixture was extracted with EtOAc (300 mL×3). The combined organic layer was dried on Na₂SO₄, filtered, and evaporated to give a yellow oil. This oil was purified by column chromatography on silica gel (petroleum ether/EtOAc 100/0 to 85/15) to give Intermediate 80 (15 g, yield: 99%) as a white solid.

### Intermediate 81

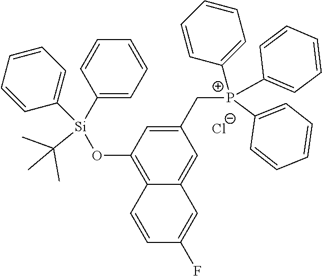 [SMILES: CC(C)(C)[Si](Oc1cc(C[P+](c2ccccc2)(c2ccccc2)c2ccccc2)cc2cc(F)ccc12)(c1ccccc1)c1ccccc1 ; [Cl-]]

A mixture of Intermediate 80 (32 g, 71.26 mmol) and PPh₃ (28 g, 2 eq.) in CH₂Cl₂ (300 mL) was concentrated under reduced pressure. The resulting residue was stirred at 140° C. for 16 h. The crude product was triturated with EtOAc and filtered to give Intermediate 81 (27 g, yield: 56%) as a white solid.

### Intermediate 82

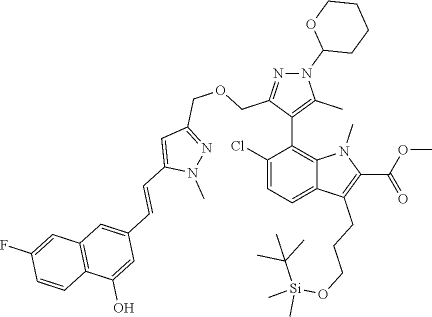 [SMILES: COC(=O)c1c(CCCO[Si](C)(C)C(C)(C)C)c2ccc(Cl)c(-c3c(COCc4cc(/C=C/c5cc(O)c6ccc(F)cc6c5)n(C)n4)nn(C4CCCCO4)c3C)c2n1C]

Sodium hydride (60% in mineral oil, 118 mg, 1.2 eq.) was added portionwise to a solution of Intermediate 81 (2.1 g, 1.2 eq.) in THF (30 mL) at 0° C. under nitrogen atmosphere. The resulting suspension was stirred for 40 min at 0° C. Then, Intermediate 79 (1.75 g, 2.46 mmol) was added and the resulting mixture was stirred at room temperature overnight. The reaction was quenched by adding water (50 mL). The mixture was extracted with EtOAc (3×100 mL). The combined organic layer was washed with water (100 mL) and brine (100 mL), dried over Na₂SO₄, filtered, and evaporated. The residue was purified by silica gel column chromatography (EtOAc/petroleum ether 0/100 to 80/100) to afford Intermediate 82 (2 g, yield: 93%) as a brown oil.

### Intermediate 83

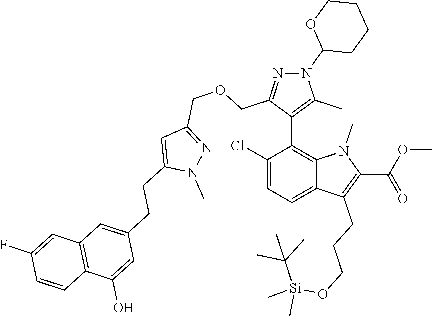 [SMILES: COC(=O)c1c(CCCO[Si](C)(C)C(C)(C)C)c2ccc(Cl)c(-c3c(COCc4cc(CCc5cc(O)c6ccc(F)cc6c5)n(C)n4)nn(C4CCCCO4)c3C)c2n1C]

Pd/C (10% 1 g, 0.08 eq.) was added to a solution of Intermediate 82 (11 g, 12.6 mmol) in EtOAc (150 mL). The reaction mixture was purged with hydrogen gas at 3.5 atm. The resulting solution was stirred at room temperature for 16 h. The reaction mixture was filtered and the filtrate was evaporated to give Intermediate 83 (10 g, yield: 57%) as a light yellow oil, used without further purification.

### Intermediate 84

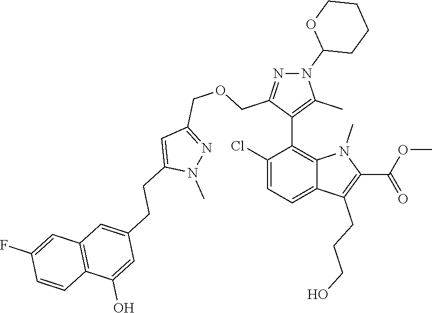 [SMILES: COC(=O)c1c(CCCO)c2ccc(Cl)c(-c3c(COCc4cc(CCc5cc(O)c6ccc(F)cc6c5)n(C)n4)nn(C4CCCCO4)c3C)c2n1C]

A solution of Intermediate 83 (7 g, 8.02 mmol) and Et₃N·(HF)₃ (CAS [73602-61-6]) (2.59 g, 2 eq.) in THF (70 mL) was stirred at room temperature for 16 h. The solvent was evaporated and the residue was partitioned between EtOAc and water. The layers were separated and the aqueous layer was extracted with EtOAc. The combined organic layer was washed with brine, dried over Na₂SO₄, filtered, and evaporated to give Intermediate 84 (6.2 g, quantitative).

### Intermediate 85

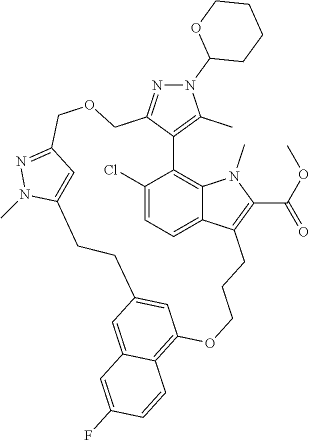 [SMILES: COC(=O)c1c2c3ccc(Cl)c(c3n1C)-c1c(nn(C3CCCCO3)c1C)COCc1cc(n(C)n1)CCc1cc(c3ccc(F)cc3c1)OCCC2]

Intermediate 84 (8 g, 10.55 mmol) and DTBAD (9.7 g, 4 eq.) were added to a solution of PPh₃ (11.1 g, 4 eq.) in toluene (100 mL). The resulting solution was stirred at 70° C. for 1 h. After cooling to room temperature, the residue was recrystallized in Et₂O (50 mL) to give Intermediate 85 (7.5 g, yield: 81%) as a light yellow oil.

### Intermediate 86 and Intermediate 87

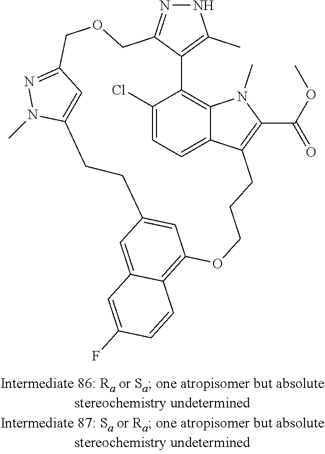 [SMILES: COC(=O)c1c2c3ccc(Cl)c(c3n1C)-c1c(n[nH]c1C)COCc1cc(n(C)n1)CCc1cc(c3ccc(F)cc3c1)OCCC2]

A solution of Intermediate 85 (8 g, 10.8 mmol) and HCl (4 M in 1,4-dioxane, 100 mL) was stirred at room temperature for 3 h. The solvent was evaporated and the residue was purified by preparative chiral SFC (Column: CHIRAL ART Cellulose-SB, 30×250 mm, 5 um; Mobile Phase A: CO₂, Mobile Phase B: IPA (1% 2 M NH₃ in MeOH)) to afford Intermediate 86 (270 mg, yield: 4%) and Intermediate 87 (270 mg, yield: 4%) both as white solids.

### Intermediate 88

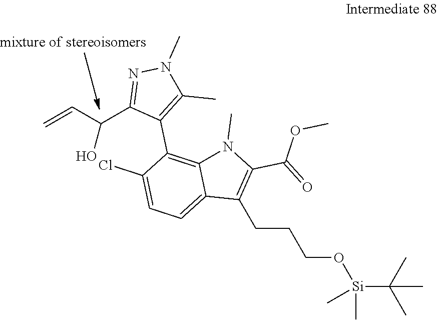 [SMILES: C=CC(O)c1nn(C)c(C)c1-c1c(Cl)ccc2c(CCCO[Si](C)(C)C(C)(C)C)c(C(=O)OC)n(C)c12]

Vinylmagnesium bromide (1 M in THF, 19.7 mL, 19.687 mmol, 1.7 eq.) was added to a solution of Intermediate 43 (6 g, 11.58 mmol) in THE (200 mL) at −10° C. The reaction mixture was stirred at −10° C. for 1 h. Aqueous NH₄Cl (50 mL) was added and the mixture was extracted with EtOAc (2×100 mL). The combined organic layer was dried with Na₂SO₄, filtered, and concentrated under vacuum. The residue was purified by flash column chromatography over silica gel (eluent: petroleum ether/EtOAc, from 100/0 to 55/45) to give Intermediate 88 (4.5 g, 31% pure, yield: 22%) as a light yellow oil.

### Intermediate 89

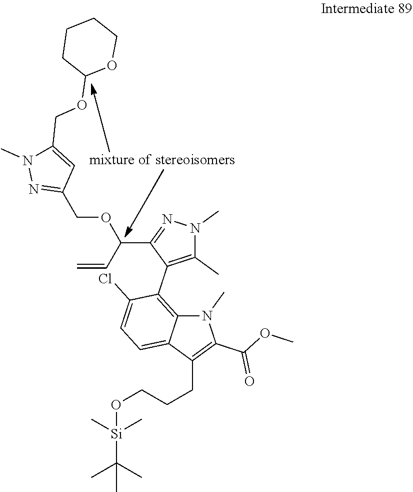

NaH (60% in mineral oil, 137 mg, 3.433 mmol, 1.5 eq.) was added to a solution of Intermediate 88 (4 g, 31% pure, 2.288 mmol) in THF (30 mL) at 0° C. The reaction mixture was stirred at 0° C. for 5 min. Intermediate 27 (1 g, 2.975 mmol, 1.3 eq.) was added and the mixture was stirred at room temperature for 1 h. The reaction mixture was poured into saturated aqueous NH₄Cl (50 mL) and was extracted with EtOAc (2×100 mL). The combined organic layer was dried with Na₂SO₄, filtered, and concentrated under vacuum. The residue was purified by flash column chromatography over silica gel (eluent: petroleum ether/EtOAc, from 100/0 to 0/100) to give Intermediate 89 (2.7 g, yield: quantitative) as a colorless oil.

### Intermediate 90

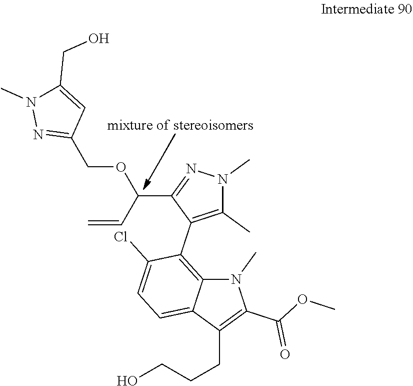

HCl (4 M in MeOH, 17.3 mL, 69.07 mmol, 20 eq.) was added to a solution of Intermediate 89 (2.68 g, 3.453 mmol) in MeOH (3 mL) and the mixture was stirred at room temperature for 30 min. The mixture was concentrated under vacuum. Aqueous NaHCO₃ (50 mL) was added to the residue and the mixture was extracted with DCM (2×100 mL). The combined organic layer was washed with brine, dried with Na₂SO₄, filtered, and concentrated. The residue was purified by flash column chromatography over silica gel (eluent: DCM/MeOH, from 100/0 to 90/10) to give Intermediate 90 (1.75 g, yield: 91%) as a white solid.

### Intermediate 91

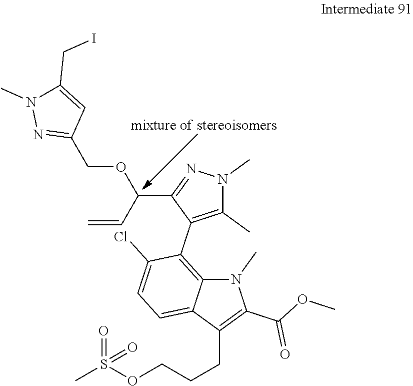

Ms₂O (2.74 g, 15.736 mmol, 5 eq.) was added to a solution of Intermediate 90 (1.75 g, 3.147 mmol) in THF (30 mL) at 0′° C. DIPEA (2.74 mL, 15.736 mmol, 5 eq.) was added and the reaction mixture was stirred at room temperature for 16 h. Lithium iodide (632 mg, 4.721 mmol, 1.5 eq.) was added at 0° C. and the reaction mixture was stirred for another 2 h. The reaction mixture was diluted with water (50 mL) and extracted with EtOAc (2×100 mL). The combined organic layer was washed with brine, dried with Na₂SO₄, filtered, and concentrated to give Intermediate 91 (2.2 g, 79% pure, yield. 74%) as a yellow oil, used without further purification.

### Intermediate 92

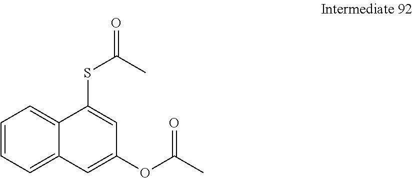 [SMILES: CC(=O)Oc1cc(SC(C)=O)c2ccccc2c1]

A solution of sodium 1-naphthol-3-sulfonate (CAS [13935-00-7], 20 g, 81.23 mmol), triphenylphosphine (76.7 g, 292.43 mmol, 3.6 eq.), and iodine (16.5 g, 64.98 mmol, 0.8 eq.) in ACN (250 mL) was stirred at 80° C. for 16 h. After cooling to room temperature, DMAP (992 mg, 8.12 mmol, 0.1 eq.) and Et₃N (22.5 mL, 162.5 mmol, 2 eq.) were added, followed by dropwise addition of Ac₂O (23 mL, 243.69 mmol, 3 eq.). The reaction mixture was stirred at room temperature for 4 h. The reaction mixture was filtered and the filtrate was evaporated. The residue was purified by column chromatography on silica gel (petroleum ether/EtOAc 100/0 to 70/30) to give Intermediate 92 (16 g, yield. 75%) as a white solid.

### Intermediate 93

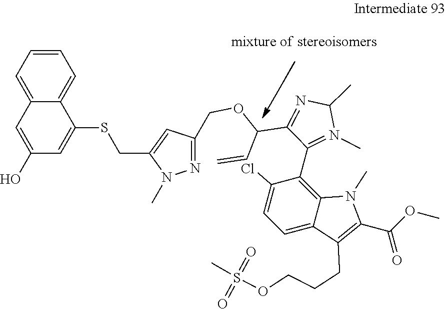

K₂CO₃ (879 mg, 6.363 mmol, 3 eq.) was added to a solution of Intermediate 91 (2 g, 2.121 mmol), Intermediate 92 (527 mg, 2.015 mmol, 0.95 eq.), and triphenylphosphine (55.6 mg, 0.212 mmol, 0.1 eq.) in MeOH (30 mL) at 0° C. The reaction mixture was stirred under nitrogen atmosphere for 1 h at room temperature. The reaction mixture was diluted with water (50 mL) and extracted with EtOAc (2×100 mL). The combined organic layer was washed with brine, dried with Na₂SO₄, filtered, and concentrated. The residue was purified by flash column chromatography over silica gel (eluent: petroleum ether/EtOAc, from 100/0 to 10/90) to give Intermediate 93 (1.2 g, yield: 59%) as a yellow solid.

### Intermediate 94

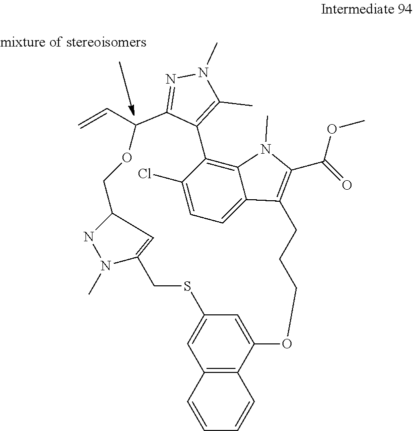 [SMILES: C=CC1OCC2C=C(CSc3cc(c4ccccc4c3)OCCCc3c(C(=O)OC)n(C)c4c(c(Cl)ccc34)-c3c1nn(C)c3C)N(C)N2]

K₂CO₃ (520 mg, 3.765 mmol, 3 eq.) was added to a solution of Intermediate 93 (1.2 g, 1.255 mmol) in ACN (20 mL) at room temperature. The reaction mixture was stirred at 80° C. for 2 h. Water (30 mL) was added and the mixture was extracted with EtOAc (2×50 mL). The combined organic layer was dried with Na₂SO₄, filtered, and concentrated under vacuum. The residue was purified by flash column chromatography over silica gel (eluent: petroleum ether/EtOAc, from 100/0 to 20/80) to give Intermediate 94 (420 mg, yield: 47%) as a yellow solid.

### Intermediate 95

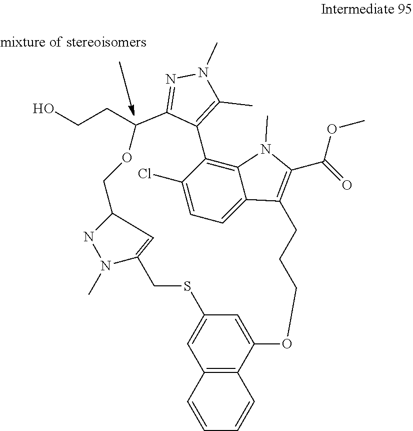 [SMILES: COC(=O)c1c2c3ccc(Cl)c(c3n1C)-c1c(nn(C)c1C)C(CCO)OCC1C=C(CSc3cc(c4ccccc4c3)OCCC2)N(C)N1]

BH₃ (1 M in THF, 2.796 mL, 2.796 mmol, 10 eq.) was added to a solution of Intermediate 94 (200 mg, 0.28 mmol) in THF (2 mL) at 0° C. and the mixture was stirred at room temperature for 2 h. Sodium perborate tetrahydrate (215 mg, 1.398 mmol, 5 eq.) and water (0.5 mL) were added and the mixture was stirred for another 72 h. Water (20 mL) was added and the mixture was extracted with EtOAc (2×50 mL). The combined organic layer was washed with brine, dried with Na₂SO₄, filtered, and concentrated. The residue was purified by HPLC (Column: Boston Green ODS 150*30 mm*5 um; A: water (0.2% formic acid)-ACN, B: ACN, A/B: 30/70 to 0/100) to give Intermediate 95 (105 mg, yield: 52%) as a white solid.

### Intermediate 96

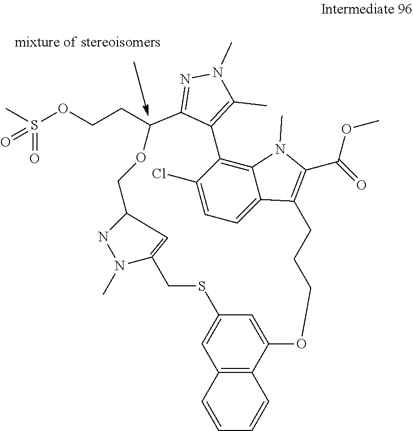

MsCl (110 mg, 0.96 mmol, 6.5 eq.) was added to a solution of Intermediate 95 (105 mg, 0.147 mmol) in DMF (5 mL) at 0° C. Et₃N (122 μL, 0.882 mmol, 6 eq.) was then added and the reaction mixture was stirred at room temperature for 2 h. The reaction mixture was diluted with saturated aqueous NaHCO₃ (5 mL) and was extracted with EtOAc (2×20 mL). The combined organic layer was washed with brine, dried with Na₂SO₄, filtered, and concentrated to give Intermediate 96 (110 mg, 88% pure, yield: 85%) as a yellow oil, used without further purification.

### Intermediate 97

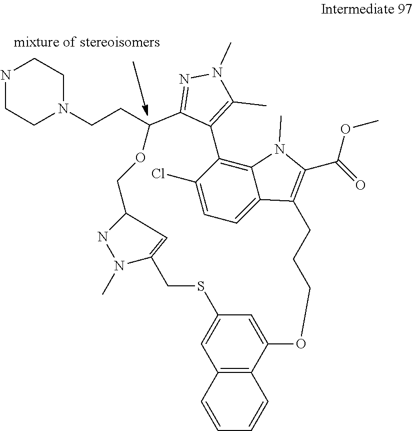 [SMILES: COC(=O)c1c2c3ccc(Cl)c(c3n1C)-c1c(nn(C)c1C)C(CCN1CCNCC1)OCC1C=C(CSc3cc(c4ccccc4c3)OCCC2)N(C)N1]

Morpholine (107 mg, 1.227 mmol, 10 eq.) was added to a solution of Intermediate 96 (110 mg, 0.123 mmol) in ACN (3 mL) at 0° C. Cs₂CO₃ (200 mg, 0.614 mmol, 5 eq.) was added and the reaction mixture was stirred at 80° C. for 2 h. The reaction mixture was diluted with water (20 mL) and extracted with EtOAc (2×20 mL). The combined organic layer was washed with brine, dried with Na₂SO₄, filtered, and concentrated. The residue was purified by HPLC (Column: Phenomenex Gemini-NX 150*30 mm*5 um; Condition: A: water (0.05% NH₃H₂O)-ACN, B: ACN, A/B: 35/65 to 5/95) to give Intermediate 97 (15 mg, yield: 15%) as a white solid.

### PREPARATION OF COMPOUNDS

### Compound 1

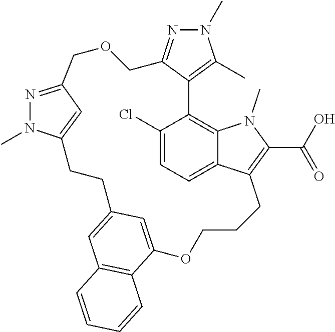 [SMILES: Cc1c2c(nn1C)COCc1cc(n(C)n1)CCc1cc(c3ccccc3c1)OCCCc1c(C(=O)O)n(C)c3c-2c(Cl)ccc13]

### Rₐ or Sₐ; One Atropisomer but Absolute Stereochemistry Undetermined

LiOH (11 mg, 8 eq.) in water (0.3 mL) was added to a solution of Intermediate 20 (37 mg, 0.057 mmol) in THE (0.5 mL) and methanol (0.5 mL). The reaction mixture was stirred at 60° C. for 2 h. The reaction mixture was cooled to room temperature and neutralised with 1 M aqueous HCl (0.46 mL, 8 eq.) and the crude mixture was concentrated. The residue was taken up in EtOAc (10 mL) and water (10 mL) and the layers were separated. The aqueous layer was extracted with EtOAc (3×30 mL), and the combined organic layer was washed with brine, dried over MgSO₄, filtered, and concentrated in vacuo to afford Compound 1 (36 mg, yield: 99%).

¹H NMR (400 MHz, DMSO-d₆, 91° C.) 6 ppm 2.00 (s, 3H) 2.26 (br s, 2H) 2.93-3.03 (m, 30H) 3.03-3.09 (m, 2H) 3.45-3.52 (m, 1H) 3.49 (s, 3H) 3.54 (s, 3H) 3.78 (s, 2H) 3.81 (s, 2H) 3.89-3.97 (m, 2H) 4.06-4.17 (m, 2H) 4.97 (s, 1H) 6.45 (s, 11H) 7.03 (d, J=8.6 Hz, 1H) 7.18 (s, 1H) 7.34-7.39 (m, 1H) 7.39-7.44 (m, 1H) 7.64 (d, J=8.6 Hz, 1H) 7.68 (d, J=7.5 Hz, 1H) 8.08 (d, J=8.2 Hz, 1H).

### Compound 2

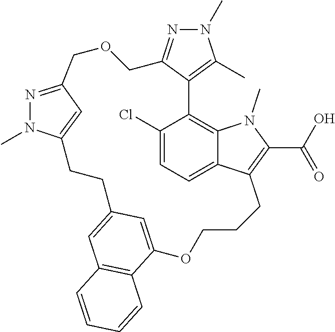 [SMILES: Cc1c2c(nn1C)COCc1cc(n(C)n1)CCc1cc(c3ccccc3c1)OCCCc1c(C(=O)O)n(C)c3c-2c(Cl)ccc13]

### Sₐ or Rₐ; One Atropisomer but Absolute Stereochemistry Undetermined

Compound 2 was prepared in a similar way as Compound 1, starting from Intermediate 21 instead of Intermediate 20.

¹H NMR (400 MHz, DMSO-d₆) δ ppm 1.98 (s, 3H), 2.26 (br s, 2H), 2.96-3.15 (m, 5H), 3.17 (d, J=5.0 Hz, 2H), 3.31 (s, 8H), 3.38-3.44 (m, 1H), 3.46 (s, 3H), 3.62 (s, 3H), 3.77-3.81 (m, 3H), 3.81-3.91 (m, 3H), 3.94-4.02 (m, 1H), 4.03-4.12 (m, 3H), 4.77 (s, 1H), 6.52 (s, 1H), 7.14 (d, J=8.6 Hz, 1H), 7.16 (s, 1H), 7.35-7.46 (m, 2H), 7.67 (d, J=7.9 Hz, 1H), 7.80 (d, J=8.7 Hz, 1H), 8.04 (d, J=8.2 Hz, 1H).

### Compound 3

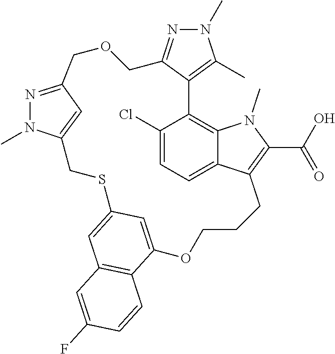 [SMILES: Cc1c2c(nn1C)COCc1cc(n(C)n1)CSc1cc(c3ccc(F)cc3c1)OCCCc1c(C(=O)O)n(C)c3c-2c(Cl)ccc13]

### Rₐ or Sₐ; One Atropisomer but Absolute Stereochemistry Undetermined

Compound 3 was prepared by using an analogous protocol as Compound 1, starting from Intermediate 25 instead of Intermediate 20.

NMR: ¹H NMR (400 MHz, CHLOROFORM-d) δ ppm 0.87 (br d, J=17.5 Hz, 2H), 1.21-1.39 (m, 4H), 2.14 (s, 3H), 2.28 (br d, J=5.5 Hz, 2H), 2.85 (br d, J=9.7 Hz, 2H), 2.93-3.03 (m, 2H), 3.17-3.24 (m, 4H), 3.49-3.52 (m, 6H), 3.56-3.58 (m, 3H), 3.58-3.69 (m, 2H), 3.78 (d, J=11.0 Hz, 1H), 3.88 (s, 3H), 3.94 (d, J=11.0 Hz, 1H), 4.17 (d, J=12.0 Hz, 1H), 4.52 (d, J=12.0 Hz, 1H), 5.52 (s, 1H), 5.73 (s, 1H), 7.11-7.16 (m, 2H), 7.21 (td, J=8.8, 2.6 Hz, 1H), 7.31 (d, J=10.1 Hz, 1H), 7.56 (d, J=8.7 Hz, 1H), 8.29 (dd, J=9.3, 5.7 Hz, 1H).

### Compound 4

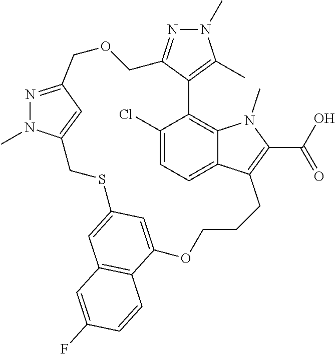 [SMILES: Cc1c2c(nn1C)COCc1cc(n(C)n1)CSc1cc(c3ccc(F)cc3c1)OCCCc1c(C(=O)O)n(C)c3c-2c(Cl)ccc13]

### Sₐ or Rₐ; One Atropisomer but Absolute Stereochemistry Undetermined

Compound 4 was prepared by using an analogous protocol as Compound 1, starting from Intermediate 26 instead of Intermediate 20.

NMR: ¹H NMR (400 MHz, CHLOROFORM-d) δ ppm 0.86 (br d, J=17.9 Hz, 1H), 1.14-1.37 (m, 3H), 2.00-2.18 (m, 3H), 2.28 (br s, 2H), 2.85 (br d, J=10.7 Hz, 2H), 2.96 (br d, J=9.9 Hz, 2H), 3.23 (br s, 4H), 3.45-3.53 (m, 1H), 3.53-3.57 (m, 3H), 3.58-3.68 (m, 2H), 3.77-3.84 (m, 1H), 3.84-3.90 (m, 3H), 3.91-3.99 (m, 1H), 4.19 (br d, J=11.7 Hz, 1H), 4.49 (br d, J=11.4 Hz, 1H), 5.47 (br s, 1H), 5.76 (br s, 1H), 7.06-7.14 (m, 2H), 7.19 (br t, J=8.7 Hz, 1H), 7.27-7.35 (m, 1H), 7.49-7.59 (m, 1H), 8.28 (br s, 1H).

### Compound 5

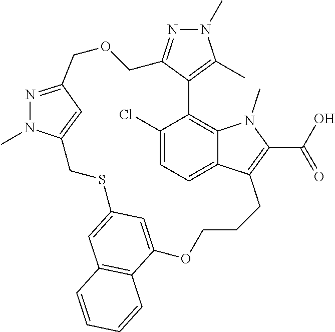 [SMILES: Cc1c2c(nn1C)COCc1cc(n(C)n1)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

A solution of LiOH (65 mg, 15 eq.) in water (2 mL) was added to a solution of Intermediate 33 (122 mg, 0.182 mmol) in THE (4 mL) and MeOH (4 mL). The reaction mixture was stirred at 60° C. for 3 h. The reaction mixture was cooled to room temperature, diluted with MeOH and directly injected onto a preparative HPLC (Stationary phase: RP XBridge Prep C18 OBD-10 μm, 30×150 mm, Mobile phase: 0.25% NH₄HCO₃ solution in water, ACN) to give a white solid. This solid was triturated with DIPE, filtered, and dried to afford Compound 5 (90 mg, yield: 75%) as a white solid.

¹H NMR (400 MHz, DMSO-d₆) δ ppm 1.98 (s, 3H), 2.28-2.38 (m, 2H), 3.05-3.15 (m, 1H), 3.39-3.48 (m, 4H), 3.78 (s, 3H), 3.79 (s, 3H), 3.82 (d, J=11.9 Hz, 1H), 3.91-4.01 (m, 2H), 4.03 (d, J=10.6 Hz, 1H), 4.07 (d, J=10.6 Hz, 1H), 4.11-4.20 (m, 1H), 4.32 (d, J=15.6 Hz, 1H), 4.41 (d, J=15.6 Hz, 1H), 4.82 (s, 1H), 6.83 (s, 1H), 7.21 (d, J=8.6 Hz, 1H), 7.30 (s, 1H), 7.37-7.49 (m, 2H), 7.65-7.69 (m, 1H), 7.89 (d, J=8.6 Hz, 1H), 8.00-8.05 (m, 1H).

### Compound 6

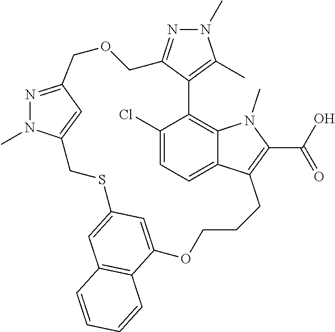 [SMILES: Cc1c2c(nn1C)COCc1cc(n(C)n1)CSc1cc(c3ccccc3c1)OCCCc1c(C(=O)O)n(C)c3c-2c(Cl)ccc13]

### Sₐ or Rₐ; One Atropisomer but Absolute Stereochemistry Undetermined

Compound 6 was prepared by using an analogous protocol as Compound 5, starting from Intermediate 34 instead of Intermediate 33.

¹H NMR (400 MHz, DMSO-d₆) δ ppm 1.98 (s, 3H), 2.28-2.38 (m, 2H), 3.05-3.15 (m, 1H), 3.39-3.48 (m, 4H), 3.78 (s, 3H), 3.79 (s, 3H), 3.82 (d, J=11.9 Hz, 1H), 3.91-4.01 (m, 2H), 4.03 (d, J=10.6 Hz, 1H), 4.06 (d, J=10.6 Hz, 1H), 4.11-4.20 (m, 1H), 4.32 (d, J=15.6 Hz, 1H), 4.40 (d, J=15.6 Hz, 1H), 4.81 (s, 1H), 6.82 (s, 1H), 7.21 (d, J=8.6 Hz, 1H), 7.30 (s, 1H), 7.37-7.49 (m, 2H), 7.64-7.70 (m, 1H), 7.90 (d, J=8.8 Hz, 1H), 7.99-8.06 (m, 1H).

### Compound 7

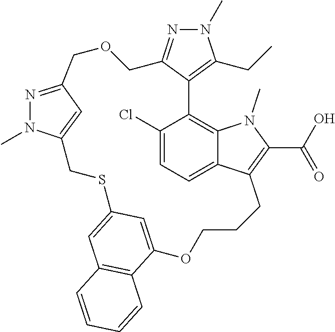 [SMILES: CCc1c2c(nn1C)COCc1cc(n(C)n1)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

Compound 7 was prepared by using an analogous protocol as Compound 5, starting from Intermediate 41 instead of Intermediate 33.

MP: 269.16° C. (DSC: From 30 to 300° C. at 10° C./min 50 mL N₂)

¹H NMR (400 MHz, DMSO-d₆) δ ppm 0.89 (t, J=7.5 Hz, 3H), 2.27-2.38 (m, 3H), 2.40-2.47 (m, 1H), 3.04-3.12 (m, 1H), 3.37-3.42 (m, 1H), 3.43 (s, 3H), 3.77 (s, 3H), 3.78-3.83 (m, 4H), 3.92-4.02 (m, 3H), 4.05 (d, J=10.6 Hz, 1H), 4.11-4.19 (m, 1H), 4.31 (d, J=15.6 Hz, 1H), 4.41 (d, J=15.6 Hz, 1H), 4.80 (s, 1H), 6.83 (s, 1H), 7.21 (d, J=8.6 Hz, 1H), 7.28 (s, 1H), 7.36-7.47 (m, 2H), 7.63-7.68 (m, 1H), 7.88 (d, J=8.6 Hz, 1H), 7.98-8.03 (m, 1H).

### Compound 8

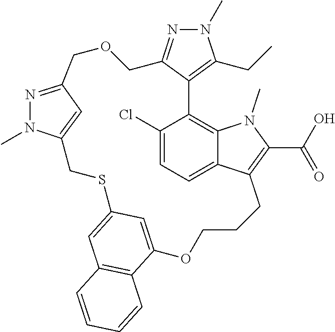 [SMILES: CCc1c2c(nn1C)COCc1cc(n(C)n1)CSc1cc(c3ccccc3c1)OCCCc1c(C(=O)O)n(C)c3c-2c(Cl)ccc13]

### Sₐ or Rₐ; One Atropisomer but Absolute Stereochemistry Undetermined

Compound 8 was prepared by using an analogous protocol as Compound 6, starting from Intermediate 42 instead of Intermediate 34.

MP: 264.21° C. (DSC: From 30 to 300° C. at 10° C./min 50 mL N₂) ¹H NMR (400 MHz, DMSO-d₆) δ ppm 0.89 (t, J=7.6 Hz, 3H), 2.27-2.38 (m, 3H), 2.39-2.48 (m, 1H), 3.03-3.11 (m, 1H), 3.37-3.41 (m, 1H), 3.43 (s, 3H), 3.77 (s, 3H), 3.78-3.83 (m, 4H), 3.92-4.03 (m, 3H), 4.05 (d, J=10.5 Hz, 1H), 4.10-4.19 (m, 1H), 4.30 (d, J=15.6 Hz, 1H), 4.43 (d, J=15.6 Hz, 1H), 4.83 (s, 1H), 6.83 (s, 1H), 7.21 (d, J=8.6 Hz, 1H), 7.28 (s, 1H), 7.35-7.47 (m, 2H), 7.62-7.68 (m, 1H), 7.87 (d, J=8.6 Hz, 1H), 7.97-8.03 (m, 1H).

### Compound 9

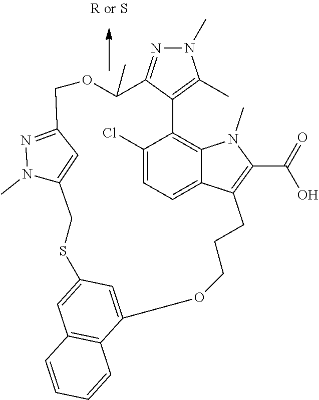 [SMILES: Cc1c2c(nn1C)C(C)OCc1cc(n(C)n1)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

LiOH (1 M in water, 1.6 mL, 20 eq.) was added to a stirred solution of Intermediate 49 (54 mg, 0.079 mmol) in MeOH (5 mL) and THF (2 mL) and the reaction mixture was stirred at 60° C. for 6 h. The turbid solution was partly concentrated in vacuo, diluted with DCM and water, and treated with 1 M HCl until pH was ˜1. The layers were separated, the aqueous layer was extracted with DCM (4×). The combined organic layer was dried over MgSO₄, filtered, and evaporated to afford a solid. This solid was dissolved in MeOH (1 mL) and water (15 mL) and this mixture was freeze-dried to afford Compound 9 (53 mg, yield: 99%) as a fluffy powder.

¹H NMR (400 MHz, CHLOROFORM-d) δ ppm 1.36-1.49 (m, 3H) 2.01-2.24 (m, 4H) 2.41 (br s, 1H) 3.25-3.40 (m, 1H) 3.56 (s, 3H) 3.63-3.89 (m, 11H) 3.92-4.01 (m, 2H) 4.12 (br d, J=11.08 Hz, 1H) 4.45 (br d, J=6.38 Hz, 1H) 5.28 (s, 1H) 6.45 (s, 1H) 6.93 (br d, J=8.47 Hz, 1H) 7.42-7.58 (m, 4H) 7.64-7.74 (m, 1H) 8.35 (br d, J=4.60 Hz, 1H).

### Compound 10

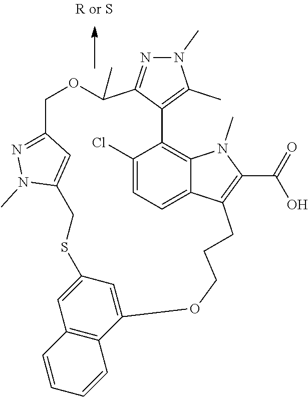 [SMILES: Cc1c2c(nn1C)C(C)OCc1cc(n(C)n1)CSc1cc(c3ccccc3c1)OCCCc1c(C(=O)O)n(C)c3c-2c(Cl)ccc13]

### Sₐ or Rₐ; One Atropisomer but Absolute Stereochemistry Undetermined

Compound 10 was prepared by using an analogous protocol as Compound 9, starting from Intermediate 50 instead of Intermediate 49.

### Compound 11

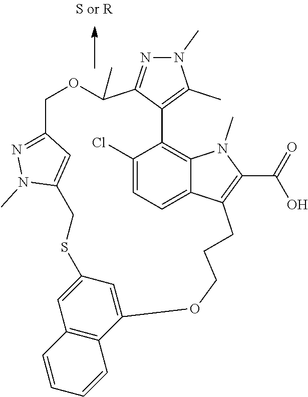 [SMILES: Cc1c2c(nn1C)C(C)OCc1cc(n(C)n1)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

Compound 11 was prepared by using an analogous protocol as Compound 9, starting from Intermediate 51 instead of Intermediate 49.

¹H NMR (400 MHz, CHLOROFORM-d) δ ppm 1.52 (d, J=6.48 Hz, 3H) 2.06 (s, 3H) 2.34 (br d, J=4.49 Hz, 2H) 3.16-3.29 (m, 1H) 3.58-3.79 (m, 10H) 3.82-4.00 (m, 7H) 4.17 (q, J=6.48 Hz, 1H) 5.16 (s, 1H) 6.26 (s, 1H) 7.14 (d, J=8.57 Hz, 1H) 7.42-7.52 (m, 3H) 7.57-7.68 (m, 2H) 8.23-8.29 (m, 1H).

### Compound 12

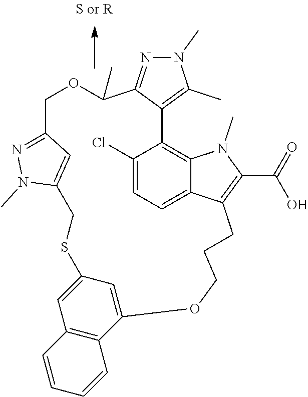 [SMILES: Cc1c2c(nn1C)C(C)OCc1cc(n(C)n1)CSc1cc(c3ccccc3c1)OCCCc1c(C(=O)O)n(C)c3c-2c(Cl)ccc13]

### Sₐ or Rₐ; One Atropisomer but Absolute Stereochemistry Undetermined

Compound 12 was prepared by using an analogous protocol as Compound 9, starting from Intermediate 52 instead of Intermediate 49.

¹H NMR (400 MHz, CHLOROFORM-d) δ ppm 1.47-1.57 (m, 3H) 2.06 (s, 3H) 2.29-2.41 (m, 2H) 3.17-3.27 (m, 1H) 3.58-3.82 (m, 11H) 3.85-4.00 (m, 6H) 4.12-4.22 (m, 1H) 5.16 (s, 1H) 6.27 (d, J=1.25 Hz, 1H) 7.14 (d, J=8.57 Hz, 1H) 7.43-7.55 (m, 3H) 7.55-7.65 (m, 2H) 8.24-8.29 (m, 1H).

### Compound 13

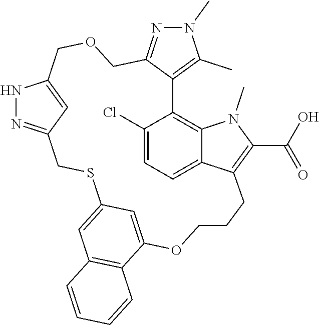 [SMILES: Cc1c2c(nn1C)COCc1cc(n[nH]1)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

Compound 13 was prepared by using an analogous protocol as Compound 1, starting from Intermediate 62 instead of Intermediate 20.

¹H NMR (400 MHz, CDCl₃) δ ppm 2.05 (s, 3H), 2.37 (br s, 2H), 3.20-3.29 (m, 1H), 3.53 (br dd, J=14.4, 6.2 Hz, 1H), 3.60 (s, 3H), 3.64-3.73 (m, 1H), 3.88 (s, 3H), 3.90-3.97 (m, 2H), 4.14-4.25 (m, 2H), 4.33 (d, J=11.0 Hz, 1H), 4.42 (d, J=12.9 Hz, 1H), 4.50 (d, J=11.0 Hz, 1H), 5.87 (s, 1H), 6.49 (s, 1H), 6.94 (d, J=8.5 Hz, 1H), 7.38 (s, 1H), 7.40-7.46 (m, 2H), 7.50 (d, J=8.6 Hz, 1H), 7.63 (d, J=6.8 Hz, 1H), 8.24-8.29 (m, 1H).

### Compound 14

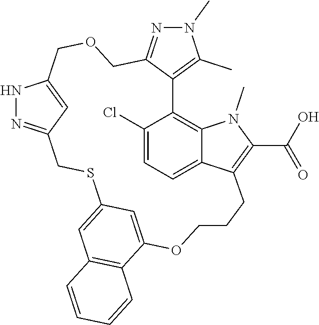 [SMILES: Cc1c2c(nn1C)COCc1cc(n[nH]1)CSc1cc(c3ccccc3c1)OCCCc1c(C(=O)O)n(C)c3c-2c(Cl)ccc13]

### Sₐ or Rₐ; One Atropisomer but Absolute Stereochemistry Undetermined

Compound 14 was prepared by using an analogous protocol as Compound 1, starting from Intermediate 63 instead of Intermediate 20.

¹H NMR (400 MHz, CHLOROFORM-d) δ ppm 2.04 (s, 3H), 2.36 (br s, 2H), 3.20-3.30 (m, 1H), 3.49-3.58 (m, 1H), 3.61 (s, 3H), 3.63-3.71 (m, 1H), 3.89 (s, 3H), 3.91-3.97 (m, 1H), 4.15-4.25 (m, 2H), 4.33 (d, J=11.1 Hz, 1H), 4.43 (d, J=12.8 Hz, 1H), 4.52 (d, J=11.1 Hz, 1H), 5.85 (br s, 1H), 6.47 (s, 1H), 6.87 (d, J=8.6 Hz, 1H), 7.35-7.52 (m, 4H), 7.61 (br d, J=5.0 Hz, 1H), 8.25-8.32 (m, 1H).

### Compound 15

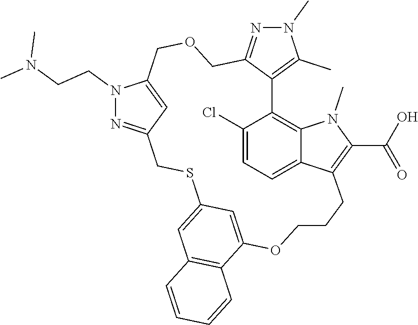 [SMILES: Cc1c2c(nn1C)COCc1cc(nn1CCN(C)C)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

Compound 15 was prepared by using an analogous protocol as Compound 5, starting from Intermediate 64 instead of Intermediate 33.

¹H NMR (400 MHz, CDCl₃) δ ppm 2.05 (s, 3H), 2.26 (s, 6H), 2.43 (br s, 1H), 3.00 (br s, 2H), 3.08-3.18 (m, 1H), 3.49 (s, 3H), 3.59 (br d, J=14.1 Hz, 1H), 3.64-3.77 (m, 2H), 3.78 (br s, 1H), 3.87 (s, 3H), 3.91 (s, 1H), 3.97 (br d, J=11.1 Hz, 2H), 4.09-4.19 (m, 2H), 4.20-4.30 (m, 3H), 5.67 (br s, 1H), 6.71 (br d, J=8.8 Hz, 1H), 6.83 (s, 1H), 7.33-7.39 (m, 2H), 7.40-7.48 (m, 2H), 7.65 (br d, J=7.4 Hz, 1H), 8.30 (br d, J=7.5 Hz, 1H).

### Compound 16

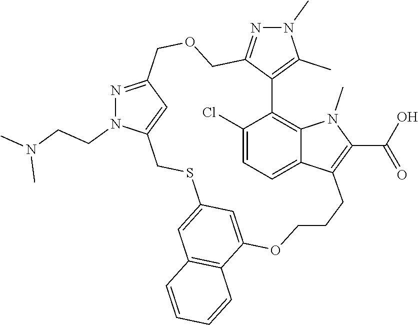 [SMILES: Cc1c2c(nn1C)COCc1cc(n(CCN(C)C)n1)CSc1cc(c3ccccc3c1)OCCCc1c(C(=O)O)n(C)c3c-2c(Cl)ccc13]

### Rₐ or Sₐ; One Atropisomer but Absolute Stereochemistry Undetermined

Compound 16 was prepared by using an analogous protocol as Compound 5, starting from Intermediate 65 instead of Intermediate 33.

¹H NMR (400 MHz, CDCl₃) δ ppm 2.06 (s, 3H), 2.42 (br s, 6H), 2.97 (br s, 1H), 3.02-3.18 (m, 2H), 3.53 (s, 3H), 3.63 (br t, J=10.8 Hz, 1H), 3.75-3.80 (m, 1H), 3.83 (s, 3H), 3.91 (d, J=11.1 Hz, 1H), 3.93-4.05 (m, 3H), 4.17 (br d, J=12.2 Hz, 2H), 4.24 (br s, 3H), 4.31 (br d, J=11.2 Hz, 2H), 5.45 (br s, 1H), 6.53 (s, 1H), 7.09-7.16 (m, 1H), 7.35 (s, 1H), 7.36-7.42 (m, 2H), 7.57 (br d, J=8.0 Hz, 2H), 8.16 (br d, J=8.0 Hz, 1H).

### Compound 17 and Compound 18

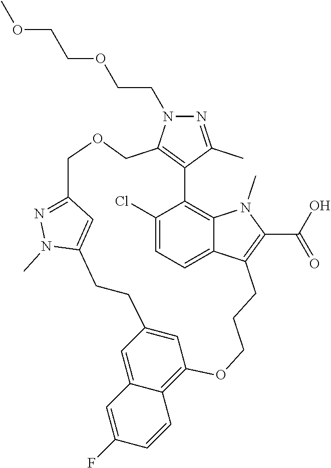 [SMILES: COCCOCCn1nc(C)c2c1COCc1cc(n(C)n1)CCc1cc(c3ccc(F)cc3c1)OCCCc1c(C(=O)O)n(C)c3c-2c(Cl)ccc13]

### Compound 17: Rₐ or Sₐ; One Atropisomer but Absolute Stereochemistry Undetermined

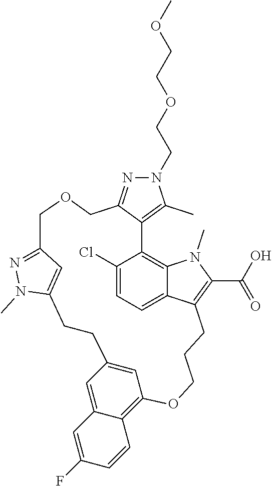 [SMILES: COCCOCCn1nc2c(c1C)-c1c(Cl)ccc3c(c(C(=O)O)n(C)c13)CCCOc1cc(cc3cc(F)ccc13)CCc1cc(nn1C)COC2]

### Compound 18: Rₐ or Sₐ; One Atropisomer but Absolute Stereochemistry Undetermined

1-Bromo-2-(2-methoxyethoxy)ethane (226 mg, 3 eq.) and Cs₂CO₃ (402 mg, 3 eq.) were added to a solution of Intermediate 86 (270 mg, 0.411 mmol) in DMF (3 mL). The reaction mixture was stirred at room temperature for 16 h and then diluted with EtOAc (5 mL). The solution was washed with water (4 mL×2) and brine (4 mL). The organic layer was dried with Na₂SO₄ and evaporated to give a light yellow solid. This solid was added to a solution of LiOH (29 mg, 3 eq.) in THF (2 mL) and water (2 mL). The resulting solution was stirred at room temperature for 16 h. The organic solvents were evaporated and the aqueous layer was extracted with EtOAc (3 mL). The aqueous layer was separated and evaporated to give a light yellow solid (200 mg) which was purified preparative SFC (column: CHIRAL ART Cellulose-SB, 30×250 mm, 5 m; Mobile Phase A: CO₂, Mobile Phase B: (hexane:DCM 3:1)(0.1% DEA):IPA 85:15) to afford Compound 17 (19 mg, yield: 6%) and Compound 18 (34 mg, yield: 11%).

### Compound 17

¹H NMR (400 MHz, CD₃OD) δ ppm 8.09 (d, J=3.56 Hz, 1H), 7.51 (d, J=8.56 Hz, 1H), 7.15-7.23 (m, 1H), 7.02-7.10 (m, 1H), 6.89 (t, J=9.22 Hz, 2H), 6.31 (s, 1H), 5.02 (s, 1H), 4.15-4.32 (m, 2H), 4.07 (s, 2H), 3.89-3.90 (m, 3H), 3.69-3.87 (m, 3H), 3.52 (s, 3H), 3.33-3.44 (m, 9H), 3.04-3.20 (m, 4H), 2.87-2.95 (m, 3H), 2.17-2.30 (m, 2H), 1.92 (s, 3H).

¹⁹F NMR (376.52 Hz, CD₃OD) δ ppm −117.50 (s, 1F).

### Compound 18

¹H NMR (400 MHz, CD₃OD) δ ppm 8.09-8.14 (m, 1H), 7.55 (d, J=8.6 Hz, 1H), 7.21 (d, J=10.28 Hz, 1H), 7.01-7.10 (m, 1H), 7.00 (s, 1H), 6.94 (d, J=8.56 Hz, 1H), 6.26 (s, 1H), 4.93 (s, 1H), 4.07-4.29 (m, 4H), 3.68-3.83 (m, 6H), 3.35-3.50 (m, 12H), 2.81-3.22 (m, 7H), 2.10-2.31 (m, 2H), 2.00 (s, 3H).

¹⁹F NMR (376.52 Hz, CD₃OD) δ ppm −117.56 (s, 1F).

### Compound 19 and Compound 20

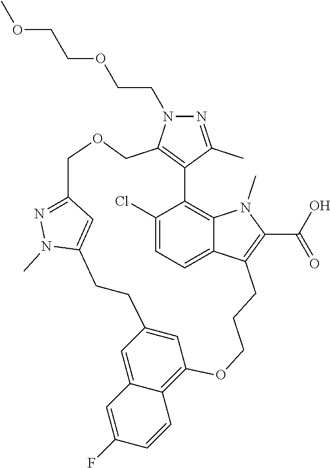 [SMILES: COCCOCCn1nc(C)c2c1COCc1cc(n(C)n1)CCc1cc(c3ccc(F)cc3c1)OCCCc1c(C(=O)O)n(C)c3c-2c(Cl)ccc13]

### Compound 19: Sₐ or Rₐ; One Atropisomer but Absolute Stereochemistry Undetermined

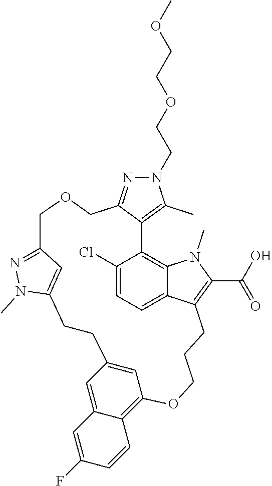 [SMILES: COCCOCCn1nc2c(c1C)-c1c(Cl)ccc3c(c(C(=O)O)n(C)c13)CCCOc1cc(cc3cc(F)ccc13)CCc1cc(nn1C)COC2]

### Compound 20: Sₐ or Rₐ; One Atropisomer but Absolute Stereochemistry Undetermined

Compound 19 and Compound 20 were prepared by using an analogous protocol as Compound 17 and Compound 18 respectively, starting from Intermediate 87 instead of Intermediate 86.

### Compound 19

¹H NMR (400 MHz, CD₃OD) δ ppm 8.07 (d, J=3.52 Hz, 1H), 7.55 (d, J=8.6 Hz, 1H), 7.11-7.19 (m, 1H), 7.01-7.10 (m, 1H), 6.89-6.91 (m, 2H), 6.26 (s, 1H), 4.98 (s, 1H), 4.12-4.35 (m, 2H), 3.88-4.03 (m, 4H), 3.65-3.87 (m, 4H), 3.35-3.52 (m, 11H), 3.00-3.20 (m, 5H), 2.78-2.98 (m, 3H), 2.20-2.32 (m, 2H), 1.92 (s, 3H).

¹⁹F NMR (376.52 Hz, CD₃OD) δ ppm −117.48 (s, 1F).

### Compound 20

¹H NMR (400 MHz, CD₃OD) δ ppm 8.08-8.12 (m, 1H), 7.55 (d, J=8.6 Hz, 1H), 7.21 (d, J=10.28 Hz, 1H), 6.93-7.10 (m, 3H), 6.26 (s, 1H), 4.92 (s, 1H), 4.07-4.30 (m, 4H), 3.72-3.83 (m, 6H), 3.35-3.49 (m, 11H), 2.81-3.16 (m, 8H), 2.13-2.32 (m, 2H), 2.00 (s, 3H).

¹⁹F NMR (376.52 Hz, CD₃OD) δ ppm −117.56 (s, 1F).

### Compound 21

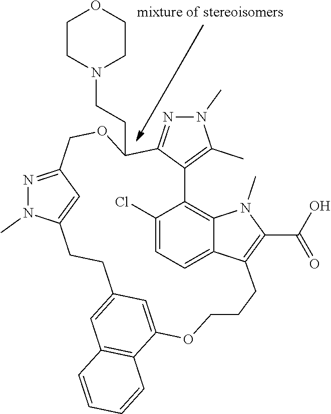 [SMILES: Cc1c2c(nn1C)C(CCN1CCOCC1)OCc1cc(n(C)n1)CCc1cc(c3ccccc3c1)OCCCc1c(C(=O)O)n(C)c3c-2c(Cl)ccc13]

LiOH (8 mg, 0.185 mmol, 10 eq.) was added to a solution of Intermediate 97 (15 mg, 0.018 mmol) in THF (1 mL), MeOH (1 mL), and water (0.2 mL) at room temperature. The reaction mixture was stirred at room temperature for 16 h. The mixture was adjusted to pH˜⁶ with HCl (1 M in water) and extracted with EtOAc (2×10 mL). The combined organic layer was dried over Na₂SO₄, filtered, and concentrated under vacuum. The residue was purified by HPLC (Column: Boston Green ODS 150*30 mm*5 um; Condition: A: water (0.2% formic acid)-ACN, B: ACN, A/B 70/30 to 40/60) to give Compound 21 (6 mg, yield: 56%) as a white solid.

¹H NMR (400 MHz, METHANOL-d4) δ ppm=8.24 (br d, J=9.0 Hz, 1H), 7.73-7.64 (m, 1H), 7.51-7.43 (m, 3H), 7.42 (s, 1H), 6.84 (d, J=8.6 Hz, 1H), 6.65 (s, 1H), 5.48 (s, 1H), 4.61 (br s, 1H), 4.38 (br d, J=15.7 Hz, 1H), 4.23 (dd, J=4.0, 8.8 Hz, 1H), 4.10-4.01 (m, 4H), 3.88 (s, 3H), 3.85-3.79 (m, 1H), 3.73 (s, 3H), 3.60 (br s, 5H), 3.36 (s, 3H), 3.18 (br t, J=9.9 Hz, 1H), 2.89 (br s, 1H), 2.79 (br s, 4H), 2.37 (br s, 1H), 2.24-2.09 (m, 5H), 2.00-1.89 (m, 1H)

### Analytical Analysis

### LCMS Methods

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min; column temperature (T) in ° C.; Run time in minutes)

| | | | | | Flow | |
|---|---|---|---|---|---|---|
| Method | | | | | Column | Run |
| code | Instrument | Column | Mobile phase | gradient | T | time |
| 1 | Waters: | Waters: | A: 10 mM | From 95% | 0.8 | 2 |
| | Acquity ® | BEH C18 | CH₃COONH₄ | A to 5% | 55 | |
| | UPLC ®- | (1.7 μm, | in 95% H₂O + | A in | | |
| | DAD and | 2.1 × 50 mm) | 5% CH₃CN | 1.3 min, | | |
| | SQD | | B: CH₃CN | held for | | |
| | | | | 0.7 min. | | |
| 2 | Waters: | Waters: | A: 10 mM | From 10%0 | 0.7 | 3.5 |
| | Acquity ® | BEH | CH₃COONH₄ | A to | 55 | |
| | UPLC ®- | (1.8 μm, | in 95% H₂O + | 5% A in | | |
| | DAD and | 2.1 × 100 mm) | 5% | 2.10 min, | | |
| | SQD | | CH₃CN | to 0% A | | |
| | | | B: CH₃CN | in 0.90 | | |
| | | | | min, | | |
| | | | | to 5% A | | |
| | | | | in 0.5 min | | |
| 3 | Waters: | Waters: BEH | A: 0.1% | From 100% | 0.6 | 3,5 |
| | Acquity ® | (1.8 μm, | NH₄HCO₃ | A to | 55 | |
| | UPLC ®- | 2.1 × 100 mm) | in 100% | 5% A in | | |
| | DAD- | | H₂O | 2.10 min, | | |
| | ELSD and | | B: MeOH | to 0% A | | |
| | SQD2 | | | in 0.9 min | | |
| | | | | to 5% A | | |
| | | | | in 0.5 min | | |
| 4 | Waters: | Waters: BEH | A: 10 mM | From 100% | 0.6 | 3.5 |
| | Acquity ® | (1.8 μm, | CH₃COONH₄ | A to 5% | 55 | |
| | UPLC ®- | 2.1 × 100 mm) | in 95% H₂O + | A in | | |
| | DAD and | | 5% CH₃CN | 2.10 min, | | |
| | SQD | | B: CH₃CN | to 0% A | | |
| | | | | in 0.90 | | |
| | | | | min, to 5% | | |
| | | | | A in | | |
| | | | | 0.5 min | | |
| 5 | Shimadzu | Poroshell | A: 10 mM | From 10% | 1.2 | 2.85 |
| | LCMS- | HPH C18 | CH₃COONH₄ | B to 95% | 40 | |
| | 2020 | (2.7 μm, | in 95% H₂O + | B in 2 | | |
| | | 3.0 × 50 mm) | 5% | min, | | |
| | | | CH₃CN | held for | | |
| | | | B: CH₃CN | 0.7 min. | | |
| 6 | Shimadzu | Poroshell | A: 10 mM | From 10% | 1.2 | 4.0 |
| | LCMS- | HPH C18 | CH₃COONH₄ | B to 60% | 40 | |
| | 2020 | (2.7 μm, | in 95% H₂O + | B in | | |
| | | 3.0 × 50 mm) | 5% CH₃CN | 3.0 min, | | |
| | | | B: CH₃CN | 60% B to | | |
| | | | | 95% B in | | |
| | | | | 0.3 min, | | |
| | | | | held for | | |
| | | | | 0.45 min. | | |
| 7 | Agilent | Waters | A: water with | 90% A | 0.8 | 10 |
| | 1200 | Xbridge- | 0.04% TFA | held for | 50 | |
| | equiped | C18 column | B: CH₃CN | 0.8 min. | | |
| | with MSD | (5 μm, | with 0.02% | Then to | | |
| | 6110 or | 2.0 × 50 mm) | TFA | 20% A | | |
| | equivalent | | | and 80% | | |
| | | | | B in 3.7 | | |
| | | | | minutes, | | |
| | | | | held for 3 | | |
| | | | | min. | | |
| | | | | Return to | | |
| | | | | 90% A in | | |
| | | | | 2 min and | | |
| | | | | hold for | | |
| | | | | 0.5 min. | | |

### LCMS Results (RT Means Retention Time)

| Com- | |
|---|---|
| pound | |
| num- | |
| ber | LCMS results |
| 1 | confirms the MW (RT: 0.91, [M + H]+ 638, LCMS Method 1) |
| 2 | confirms the MW (RT: 0.90, [M + H]+ 638, LCMS Method 1) |
| 3 | confirms the MW (RT: 0.93, [M + H]+ 656, LCMS Method 1) |
| 4 | confirms the MW (RT: 0.89, [M + H]+ 656, LCMS Method 1) |
| 5 | confirms the MW (RT: 1.77, [M + H]+ 656, LCMS Method 2) |
| 6 | confirms the MW (RT: 1.77, [M + H]+ 656, LCMS Method 2) |
| 7 | confirms the MW (RT: 1.84, [M + H]+ 670, LCMS Method 2) |
| 8 | confirms the MW (RT: 1.84, [M + H]+ 670, LCMS Method 2) |
| 9 | confirms the MW (RT: 2.32, [M + H]+ 670, LCMS Method 3) |
| 10 | confirms the MW (RT: 2.30, [M + H]+ 670, LCMS Method 3) |
| 11 | confirms the MW (RT: 2.36, [M + H]+ 670, LCMS Method 3) |
| 12 | confirms the MW (RT: 2.37, [M + H]+ 670, LCMS Method 3) |
| 13 | confirms the MW (RT: 0.91, [M + H]+ 642, LCMS Method 1) |
| 14 | confirms the MW (RT: 0.91, [M + H]+ 642, LCMS Method 1) |
| 15 | confirms the MW (RT: 0.99, [M + H]+ 713, LCMS Method 1) |
| 16 | confirms the MW (RT: 1.93, [M + H]+ 713, LCMS Method 4) |
| 17 | confirms the MW (RT: 1.47, [M + H]+ 744, LCMS Method 5) |
| 18 | confirms the MW (RT: 2.55, [M + H]+ 744, LCMS Method 6) |
| 19 | confirms the MW (RT: 1.46, [M + H]+ 744, LCMS Method 5) |
| 20 | confirms the MW (RT: 1.46, [M + H]+ 744, LCMS Method 5) |
| 21 | confirms the MW (RT: 3.67, [M + H]+ 769, LCMS Method 7) |

### SFC Methods:

The SFC measurement was performed using an Analytical Supercritical fluid chromatography (SFC) system composed by a binary pump for delivering carbon dioxide (CO2) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Analytical SFC-MS Methods (Flow expressed in mL/min; column temperature (Col T) in ° C.; Run time in minutes, Backpressure (BPR) in bars. “iPrNH₂” means isopropylamine, “iPrOH” means 2-propanol, “EtOH” means ethanol, “min” mean minutes.

### SFC Methods:

| | | | | | Run |
|---|---|---|---|---|---|
| Method | | mobile | | Flow/ | time/ |
| code | column | phase | gradient | Col T | BPR |
| 1 | Daicel | A: CO₂ | 10%-50% | 2.5/ | 9.5/ |
| | Chiralpak ® | B: | B in 6 min, | 40 | 110 |
| | AD-H | EtOH—iPrOH + | hold 3.5 | | |
| | column (3.0 μm, | 0.2% iPrNH₂ | min | | |
| | 150 × 4.6 mm) | | | | |
| 2 | Daicel | A: CO₂ | 5% B | 2.5/ | 9.5/ |
| | Chiralpak ® | B: EtOH + 0.2% | hold 6 | 40 | 130 |
| | AD3 | iPrNH₂ | min, to | | |
| | column (3.0 μm, | | 50% | | |
| | 150 × 4.6 mm) | | in 1 min | | |
| | | | hold 2.5 | | |
| | | | min | | |
| 3 | Daicel | A: CO₂ | 10%-50% | 2.5/ | 9.5/ |
| | Chiralpak ® IC3 | B: EtOH + 0.2% | B in 6 min, | 40 | 130 |
| | column (3.0 μm, | iPrNH₂ | hold 3.5 | | |
| | 150 × 4.6 mm) | | min | | |
| 4 | Daicel | A: CO₂ | 10%-50% | 2.5/ | 9.5/ |
| | Chiralpak ® AS3 | B: EtOH + 0.2% | B in 6 min, | 40 | 130 |
| | column (3.0 μm, | iPrNH₂ | hold 3.5 | | |
| | 150 × 4.6 mm) | | min | | |
| 5 | Daicel | A: CO₂ | 10%-50% | 2.5/ | 9.5/ |
| | Chiralpak ® | B: EtOH + 0.2% | B in 6 min, | 40 | 110 |
| | AD-H | iPrNH₂ | hold 3.5 | | |
| | column (3.0 μm, | | min | | |
| | 150 × 4.6 mm) | | | | |
| 6 | CHIRALPAK | A: CO₂ | 35%-35% | 2/35 | 3.0/ |
| | IG-3, 3.0 × 50 | B: IPA + 0.1% | B in 3 min | | 103 |
| | mm, 3 μm | DEA | | | |
| 7 | CHIRALPAK | A: CO₂ | 50%-50% | 2/35 | 3.0/ |
| | IG-3, 3.0 × 50 | B: IPA + 0.1% | B in 3 min | | 103 |
| | mm, 3 μm | DEA | | | |

TABLE
Analytical SFC data-Rₜ means retention time (in minutes), [M + H]⁺
means the protonated mass of the compound, method refers to the
method used for (SFC)MS analysis of enantiomerically pure
compounds. No. means number.

| Compound | | | |
|---|---|---|---|
| No. | SFC Method | Rt | [M + H]⁺ |
| 1 | 1 | 5.84 | 638 |
| 2 | 1 | 6.94 | 638 |
| 3 | 2 | 4.50 | 656 |
| 4 | 2 | 5.22 | 656 |
| 9 | 3 | 2.75 | 670 |
| 10 | 3 | 3.24 | 670 |
| 11 | 3 | 3.25 | 670 |
| 12 | 3 | 3.19 | 670 |
| 13 | 4 | 4.54 | 642 |
| 14 | 4 | 4.09 | 642 |
| 15 | 5 | 5.52 | 713 |
| 16 | 5 | 5.34 | 713 |
| 17 | 6 | 0.98 | 744 |
| 18 | 7 | 0.68 | 744 |
| 19 | 6 | 1.54 | 744 |
| 20 | 7 | 1.17 | 744 |

### NMR

¹H NMR and ¹⁹F NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

### Pharmacological Analysis

### Biological Example 1

Terbium labeled Myeloid Cell Leukemia 1 (Mcl-1) homogeneous time-resolved fluorescence (HTRF) binding assay utilizing the BIM BH3 peptide (H2N—(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

### Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1× assay buffer, where the following ingredients were added fresh to stock buffer: 2 mM dithiothreitol (DTT), 0.0025% Tween-20, 0.1 mg/mL bovine serum albumin (BSA). The 1×Tb-Mcl-1+Cy5 Bim peptide solution was prepared by diluting the protein stock solution using the 1×assay buffer (b) to 25 pM Tb-Mcl-1 and 8 nM Cy5 Bim peptide.

Using the Acoustic ECHO, 100 nL of 100×test compound(s) were dispensed into individual wells of a white 384-well Perkin Elmer Proxiplate, for a final compound concentration of 1×and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1× Tb-Mcl-1+Cy5 Bim peptide solution. The plate was centrifuged with a cover plate at 1000 rpm for 1 minute, then incubated for 60 minutes at room temperature with plates covered. The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

### Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

35% inhibition=(((NC−IC)−(compound−IC))/(NC−IC))*100

where IC (inhibitor control, low signal)=mean signal of 1× Tb-MCl-1+Cy5 Bim peptide+ inhibitor control or 100% inhibition of Mcl-1; NC (neutral control, high signal)=mean signal 1× Tb-MCl-1+Cy5 Bim peptide with DMSO only or 0% inhibition An 11-point dose response curve was generated to determine IC₅₀ values (using GenData) based on the following equation:

Y=Bottom+(Top−Bottom)/(1+10\hat{ }((log IC₅₀−X)*HillSlope))

where Y=% inhibition in the presence of X inhibitor concentration; Top=100% inhibition derived from the IC (mean signal of Mcl-1+ inhibitor control); Bottom=0% inhibition derived from the NC (mean signal of Mcl-1+ DMSO); Hillslope=Hill coefficient; and IC₅₀=concentration of compound with 50% inhibition in relation to top/neutral control (NC).

Ki=IC₅₀/(1+[L]/Kd)

In this assay [L]=8 nM and Kd=10 nM Representative compounds of the present invention were tested according to the procedure as described above, with results as listed in the Table below (n.d. means not determined).

| Compound | Tb-MCL1 Kᵢ (nM) |
|---|---|
| 1 | 0.047 |
| 2 | 0.444 |
| 3 | 0.020 |
| 4 | 2.71 |
| 5 | 0.057 |
| 6 | 11.68 |
| 7 | 0.055 |
| 8 | 8.12 |
| 9 | 0.241 |
| 10 | 19.83 |
| 11 | 33.09 |
| 12 | 0.036 |
| 13 | 0.128 |
| 14 | 21.59 |
| 15 | 0.111 |
| 16 | 0.072 |
| 17 | 0.025 |
| 18 | 0.020 |
| 19 | 1.34 |
| 20 | 4.14 |
| 21 | 0.241 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and -7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal. This assay uses the MOLP-8 human multiple myeloma cell line, which is sensitive to MCL-1 inhibition.

### Materials:

Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| Product | Units | | Storage |
|---|---|---|---|
| RPMI1640 (no L-Glutamine, no | 500 | mL | 4° C. |
| phenol red) | | | |
| Foetal Bovine Serum (FBS) (Heat | 500 | mL | 4° C. |
| inactivated) | | | |
| L-Glutamine (200 mM) | 100 | ml | −20° C. |
| Gentamicin (50 mg/ml) | 100 | mL | 4° C. |
| Caspase 3/7 Detection kit | 100 | mL | −20° C. |
| | 10 × 10 | mL | |

### Cell Culture Media:

MOLP8

| | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |

Assay media

| | | |
|---|---|---|
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

### Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

Caspase-Glo reagent:

The assay reagent was prepared by transferring the buffer solution to the substrate vial and mixing. The solution may be stored for up to 1 week at 4° C. with negligible loss of signal.

### Assay Procedure:

Compounds were delivered in assay-ready plates (Proxiplate) and stored at −20° C. Assays always include 1 reference compound plate containing reference compounds. The plates were spotted with 40 nL of compounds (0.5% DMSO final in cells; serial dilution; 30 pM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 pM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

$$\begin{matrix}LC = median of the Low Control values \\ = Central Reference in Screener \\ = DMSO \\ = 0 \%\end{matrix}$$

$$\begin{matrix}HC = Median of the High Control values \\ = Scale Reference in Screener \\ = 30 µM of positive control \\ = 100 \% apoptosis induction\end{matrix}$$

$$\% Effect ( {AC}_{50} ) = 100 - ( ( sample - LC ) / ( HC - LC ) ) * 100$$

$$\% Control = ( sample / HC ) * 100$$

$$\% Control min = ( ( sample - LC ) / ( HC - LC ) ) * 100$$

TABLE
Measured AC₅₀ for Representative Compounds of
Formula (I). Averaged values are reported over all
runs on all batches of a particular compound.

| | MOLP8 Caspase- |
|---|---|
| Compound | Glo AC₅₀ (nM) |
| 1 | 83.3 |
| 2 | 953.7 |
| 3 | 13.5 |
| 4 | 1978.3 |
| 5 | 83.7 |
| 6 | 6642.0 |
| 7 | 83.7 |
| 8 | 5376.5 |
| 9 | 246.1 |
| 10 | 14657 |
| 11 | 18097 |
| 12 | 59.4 |
| 13 | 411.1 |
| 14 | 18030 |
| 15 | 387.5 |
| 16 | 185.0 |
| 17 | 7.4 |
| 18 | 17.6 |
| 19 | 626.3 |
| 20 | 3454.6 |
| 21 | 2156.4 |

1. A compound of Formula (I): 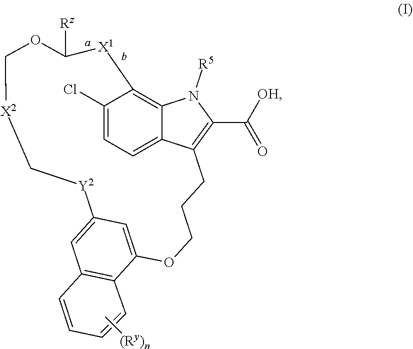
or a tautomer or a stereoisomeric form thereof, wherein:
X¹ is 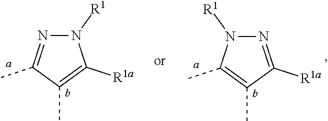
wherein ‘a’ and ‘b’ indicate how variable X¹ is attached to the remainder of the molecule;
R^{y} is halo;
n is 0, 1 or 2;
R^{z} is hydrogen; or C₁₋₄alkyl optionally substituted with one Het¹;
X² is 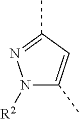
which can be attached to the remainder of the molecule in both directions;
R¹ is hydrogen; Het^{a}; C₃₋₆cycloalkyl; or C₁₋₆alkyl optionally substituted with one or two substituents that are Het¹, —OR³, or —NR^{4a}R^{4b};
R² is hydrogen; methyl; or C₂₋₆alkyl optionally substituted with one substituent that is Het¹, —OR³, or —NR^{4a}R^{4b};
R^{1a} is methyl or ethyl;
R³ is hydrogen, C₁₋₄alkyl, or —C₂₋₄alkyl-O—C₁₋₄alkyl;
R^{4a} and R^{4b} are each, independently, hydrogen or C₁₋₄alkyl;
R⁵ is methyl; or C₂₋₆alkyl optionally substituted with one substituent that is C₃₋₆cycloalkyl, Het¹, —NR^{4a}R^{4b}, or —OR³;
Het¹ represents is a 4- to 7-membered monocyclic fully saturated heterocyclyl containing one or two heteroatoms that are each, independently, O, S, or N, wherein said S-atom is optionally substituted to form S(═O) or S(═O)₂;
wherein said heterocyclyl is optionally substituted with one or two substituents that are each, independently, halo, cyano, or —O—C₁₋₄alkyl;
Het^{a} is a C-linked 4- to 7-membered monocyclic fully saturated heterocyclyl containing one heteroatom that is O, S, and N, wherein said S-atom is optionally substituted to form S(═O) or S(═O)₂, and wherein said N-atom is optionally substituted with one C₁₋₄alkyl;
Y² is —CH₂— or —S—;
or a pharmaceutically acceptable salt, or a solvate thereof.

2. The compound according to claim 1, wherein:
n is 0 or 1;
R^{z} is hydrogen; or C₁₋₄alkyl optionally substituted with one Het¹;
R¹ is C₁₋₆alkyl optionally substituted with one —OR³ substituent;
R² is hydrogen; methyl; or C₂₋₆alkyl optionally substituted with one —NR^{4a}R^{4b} substituent;
R³ is —C₂₋₄alkyl-O—C₁₋₄alkyl;
R^{4a} and R^{4b} are each, independently, C₁₋₄alkyl;
R⁵ is methyl;
Het¹ is a 4- to 7-membered monocyclic fully saturated heterocyclyl containing one or two heteroatoms each, independently, O, S, or N.

3. The compound according to claim 2, wherein R^{z} is hydrogen or C₁₋₄alkyl.

4. The compound according to claim 2, wherein n is 0.

5. The compound according to claim 2, wherein n is 1.

6. The compound according to claim 1, wherein Y² is —S—.

7. The compound according to claim 1, wherein R⁵ is methyl.

8. The compound according to claim 5, wherein R^{y} is fluoro.

9. A pharmaceutical composition comprising a compound of claim 1 and a pharmaceutically acceptable carrier or diluent.

10. A process for preparing a pharmaceutical composition of claim 9 comprising mixing a pharmaceutically acceptable carrier with a therapeutically effective amount of the compound.

11-12. (canceled)

13. The method of claim 14, wherein the cancer is prostate cancer, lung cancer, pancreatic cancer, breast cancer, ovarian cancer, cervical cancer, melanoma, B-cell chronic lymphocytic leukemia (CLL), acute myeloid leukemia (AML), or acute lymphoblastic leukemia (ALL).

14. A method of treating or preventing cancer, comprising administering to a subject in need thereof, a therapeutically effective amount of the compound of claim 1.

15. A method of treating or preventing cancer, comprising administering to a subject in need thereof, a therapeutically effective amount of the pharmaceutical composition of claim 9.

16. The compound according to claim 3, wherein n is 0.

17. The compound according to claim 3, wherein n is 1.
